(19) United States
(12) Patent Application Publication
(10) Pub. No.: US 2023/0089926 A1
(43) Pub. Date: Mar. 23, 2023

(54) Antibodies Conjugated with Fatty Acid Molecules and Uses Thereof

(71) Applicant: Phanes Therapeutics, Inc., San Diego, CA (US)

(72) Inventors: Jack Chongyang Li, San Diego, CA (US); Haiqun Jia, San Diego, CA (US); Hui Zou, San Diego, CA (US); Minghan Wang, San Diego, CA (US)

(21) Appl. No.: 17/760,394

(22) PCT Filed: Feb. 25, 2021
(86) PCT No.: PCT/US2021/019583

§ 371 Date: Aug. 9, 2022

Related U.S. Application Data
(60) Provisional application No. 62/982,476, filed on Feb. 27, 2020.

(51) Int. Cl. A61K 47/54; C12N 15/62; C12N 15/85; C12N 5/00; A61K 47/64; A61P 35/00

(52) U.S. Cl. CPC A61K 47/542; C12N 15/62; C12N 15/85; C12N 5/00; A61K 47/643; A61P 35/00

(57) ABSTRACT

Monoclonal antibodies (mAbs) or bispecific antibodies (bsAbs) or multi-specific antibodies comprising a fatty acid (FA) molecule conjugated to or near the antigen-binding domain are described. Also described are nucleic acids encoding the antibodies, compositions comprising the antibodies, and methods of producing the antibodies and using the antibodies for treating or preventing diseases, such as cancer and/or associated complications.

### CROSS REFERENCE TO RELATED APPLICATION

This application claims priority to U.S. Provisional Application No. 62/982,476, filed on Feb. 27, 2020. This disclosure is incorporated herein by reference in its entirety.

### FIELD OF THE INVENTION

This invention relates to monoclonal isolated antibodies or antigen-binding fragments thereof, wherein the monoclonal antibody or antigen-binding fragment thereof comprises (a) a heavy chain variable region (VH); and a light chain variable region (VL); wherein the monoclonal antibody or antigen-binding fragment thereof is capable of specific binding to a target antigen; wherein an amino acid residue in the VH, VL, or within a twenty (20)-amino acid distance, preferably a five (5)-amino acid distance, from the VH or VL is substituted with an amino acid residue capable of being conjugated to a fatty acid (FA); and wherein upon conjugation with the FA at the substituted amino acid residue, the monoclonal antibody or antigen-binding fragment thereof is still capable of specific binding to the target antigen; and wherein the FA-conjugated monoclonal antibody or antigen-binding fragment thereof has reduced or eliminated specific binding to the target antigen in the presence of physiological levels of albumin (e.g., 35 to 50 mg/mL). This invention also relates to multi-specific antibodies or antigen-binding fragments thereof, wherein the multi-specific antibody or antigen-binding fragment thereof comprises one or more antigen-binding arm(s) comprising a substituted amino acid residue that is conjugated to a FA. This invention also relates to nucleic acids and expression vectors encoding the antibodies, recombinant cells containing the vectors, and compositions comprising the antibodies. Methods of making the antibodies, methods of conjugating the antibodies with FAs, methods of making the compositions comprising the conjugated antibodies, and methods of using the conjugated antibodies to treat cancer are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065799.32WO1 Sequence Listing” and a creation date of Feb. 22, 2021 and having a size of 29 kb. The sequence listing submitted via EFS-Web is part of the specification and is herein incorporated by reference in its entirety.

### BACKGROUND OF THE INVENTION

T cell engagers are molecules consisting of two binding domains with one domain binding to a tumor-associated antigen (TAA) expressed on the surface of a cancer cell, and the other domain binding to a T cell surface molecule to activate the T cell. Although various T cell binding domains have been used as the activating component, anti-CD3 binding domains have been widely used as part of T cell engagers. Anti-CD3 bispecific antibodies have been used as T cell-engaging immunotherapeutic agents for recruiting T cells to tumor cells to facilitate cancer killing. One major issue with this immuno-oncology approach is the risk of cytokine release syndrome (CRS). Modulating the activities of these agents in T cell activation on sites away from the tumor microenvironment can help reduce the risk of CRS and other toxicities.

Fatty acids (FAs) exist at high concentrations in the circulating blood. Due to the hydrophobic nature, fatty acids bind to blood albumin molecules which are in the range of 35-50 mg/mL (Peters, T., 1996. All About Albumin: Biochemistry, Genetics and Medical Applications. San Diego, Calif.: Academic Press Limited). Seven common FA binding sites have been identified on albumin (Bhattacharya et al., J Mol Biol. 2000. 303:721-32; Petitpas et al., J Mol Biol. 2001.314:955-60). Additionally, it has been proposed that tumors use albumin as an energy source to support their aggressive growth (Merlot et al., Front Physiol. 2014. 5:299), which is consistent with the high rate of albumin catabolism on tumor sites (HRADEC, Br J Cancer. 1958. 12:290-304; Andersson et al., J Surg Res. 1991. 50:156-62; Schilling et al., Int J Rad Appl Instrum B. 1992. 19:685-95; Stehle et al., Crit Rev Oncol Hematol. 1997. 26:77-100). The high turnover of albumin on tumor sites suggests that there is reduced albumin concentration in the tumor microenvironment. Thus, the albumin level around certain tumor cells is expected to be lower than those in the circulating blood. Further, it has been reported that the interstitial albumin concentrations are significantly lower in adipose tissue and in skeletal muscle compared with the serum concentration (Ellmerer et al., Am. J. Physiol. Endocrinol. Metab. 278:E352-E356 (2000)).

Leveraging the lower albumin levels in tissues, such as adipose tissue, skeletal muscle, and the tumor microenvironment, compared with the circulating blood, one can use the binding of FA to albumin to modulate the activity of therapies targeting tissues or sites with low albumin levels. This can be carried out with a FA conjugated to the active site of the therapy so that when albumin is bound to the FA, the activity of the therapy is reduced or eliminated. When this approach is applied to immuno-oncology therapies, such as anti-CD3 monoclonal and/or bispecific antibodies, it has the potential to reduce the risk of CRS and other toxicities in methods of treating cancer.

### BRIEF SUMMARY OF THE INVENTION

In one general aspect, the invention relates to an isolated monoclonal antibody or antigen-binding fragment thereof, wherein the antibody or antigen-binding fragment thereof comprises (a) a heavy chain variable region (VH); and a light chain variable region (VL); wherein the antibody or antigen-binding fragment thereof binds to a target antigen, preferably a human target antigen; wherein an amino acid residue in the VH, VL, or within a twenty (20)-amino acid distance, preferably a five (5)-amino acid distance, of the VH or VL is substituted with an amino acid residue that is conjugated to a fatty acid (FA); and wherein upon conjugation with the FA at the substituted amino acid residue, the antibody or antigen-binding fragment thereof still binds to the target antigen; and wherein the FA-conjugated antibody or antigen-binding fragment thereof has reduced or eliminated specific binding to the target antigen in the presence of physiological levels of albumin (e.g., 35 to 50 mg/mL). The substituted amino acid residue can, for example, be a cysteine residue or a lysine residue or a modified amino acid that is suitable for chemical conjugation.

In certain embodiments, the substituted amino acid occurs at an amino acid residue corresponding to residue 25, 27, 62, 64, 73, 76, 101, 112, or 113 of SEQ ID NO:1 or an amino acid residue corresponding to residue 26, 27, 52, 53, 56, or 67 of SEQ ID NO:2, preferably the substitution is selected from a substitution corresponding to S25C, Y27C, K62C, K64C, K73C, S76C, D101C, S112C, or S113C of SEQ ID NO:1 or a substitution corresponding to S26C, S27C, S52C, K53C, S56C, or S67C of SEQ ID NO:2, wherein the residues are numbered according to Kabat. In certain embodiments, the substituted amino acid is at residue 64 corresponding to SEQ ID NO:1 or residue 26 corresponding to SEQ ID NO:2, preferably the substitution is selected from a K64C substitution corresponding to SEQ ID NO:1 or a S26C substitution corresponding to SEQ ID NO:2, wherein the residues are numbered according to Kabat.

In certain embodiments, the substituted amino acid occurs at residue 119 or 120 of SEQ ID NO: 9, 10, 11, or 12, or residue 121 or 124 of SEQ ID NO: 13 or 14, preferably the substitution is selected from a S119C or T120C of SEQ ID NO: 9, 10, 11, or 12, or a S121C or Q124C of SEQ ID NO: 13 or 14, wherein the residues are numbered according to EU numbering. In certain embodiments, the substituted amino acid is at residue 120 of SEQ ID NO: 9, 10, 11, or 12, preferably the substitution is a T120C substitution, wherein the residues are numbered according to the EU numbering.

In certain embodiments, the isolated monoclonal antibody or antigen-binding fragment thereof is an anti-immune cell modulator (ICM) antibody or antigen-binding fragment thereof and is capable of specific binding to the ICM, preferably a human ICM. The ICM can, for example, be selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules.

In certain embodiments, the anti-ICM antibody or antigen-binding fragment thereof is an anti-CD3 antibody or antigen-binding fragment thereof and is capable of specific binding to CD3, preferably human CD3. The isolated anti-CD3 antibody or antigen-binding fragment thereof can, for example, comprise a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:3, 4, 5, 6, 7, and 8, respectively, or SEQ ID NOs:33, 34, 35, 36, 37, and 38, respectively.

In certain embodiments, the substituted amino acid occurs at residue 25, 27, 62, 64, 73, 76, 101, 112, or 113 in the VH of the anti-CD3 mAb (SEQ ID NO:1 or SEQ ID NO:27) or 26, 27, 52, 53, 56, or 67 in the VL of the anti-CD3 mAb (SEQ ID NO:2 or SEQ ID NO:28), preferably the substitution is selected from a S25C, Y27C, K62C, K64C, K73C, S76C, D101C, S112C, or S113C in the VH (SEQ ID NO:1 or 27) or a S26C, S27C, S52C, K53C, S56C, or S67C in the VL (SEQ ID NO:2 or 28), wherein the residues are numbered according to Kabat. In certain embodiments, the substituted amino acid is at residue 64 in the VH (SEQ ID NO:1 or 27) or 26 in the VL (SEQ ID NO:2 or 28), preferably the substitution is selected from a K64C substitution in the VH (SEQ ID NO:1 or 27) or a S26C substitution in the VL (SEQ ID NO:2 or 28), wherein the residues are numbered according to Kabat.

The isolated anti-CD3 antibody or antigen-binding fragment thereof can, for example, comprise a VH region having a polypeptide sequence of SEQ ID NO:1 with an amino acid substitution of K64C and a VL region having a polypeptide sequence of SEQ ID NO:2; or a VH region having a polypeptide sequence of SEQ ID NO:27 with an amino acid substitution of K64C and a VL region having a polypeptide sequence of SEQ ID NO:28; or a VH region having a polypeptide sequence of SEQ ID NO:1 and a VL region having a polypeptide sequence of SEQ ID NO:2 with an amino acid substitution of S26C; or a VH region having a polypeptide sequence of SEQ ID NO:27 and a VL region having a polypeptide sequence of SEQ ID NO:28 with an amino acid substitution of S26C; or a heavy chain constant domain 1 (CH1) region having a polypeptide sequence selected from SEQ ID NO: 9, 10, 11 or 12 with an amino acid substitution of T120C and a light chain constant domain (CL) region having a polypeptide sequence selected from SEQ ID NO:13 or 14; or a VH region having a polypeptide sequence of SEQ ID NO:1, a VL region having a polypeptide sequence of SEQ ID NO:2, a CH1 region having a polypeptide sequence selected from SEQ ID NO: 9, 10, 11 or 12 with an amino acid substitution of T120C, and a CL region having a polypeptide sequence selected from SEQ ID NO:13 or 14; or a VH region having a polypeptide sequence of SEQ ID NO:27, a VL region having a polypeptide sequence of SEQ ID NO:28, a CH1 region having a polypeptide sequence selected from SEQ ID NO: 9, 10, 11 or 12 with an amino acid substitution of T120C, and a CL region having a polypeptide sequence selected from SEQ ID NO:13 or 14.

In certain embodiments, provided is an isolated multi-specific antibody or antigen-binding fragment thereof, wherein the multi-specific antibody or antigen-binding fragment thereof comprises the monoclonal antibody or antigen-binding fragment thereof of the invention, and wherein the multi-specific antibody or antigen-binding fragment thereof comprises one or more antigen-binding arm(s) comprising a substituted amino acid residue that is conjugated to a FA. The multi-specific antibody or antigen-binding fragment thereof can, for example, be a bispecific antibody or antigen-binding fragment thereof.

In certain embodiments, the bispecific antibody or antigen-binding fragment thereof comprises a first antigen-binding arm (Ab1) and a second antigen-binding arm (Ab2), wherein Ab1 and/or Ab2 comprises a substituted amino acid that is conjugated to a FA.

In certain embodiments, Ab1 binds an immune cell modulator (ICM), preferably a human ICM. The ICM can, for example, be selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules. In certain embodiments, the ICM is CD3, preferably human CD3.

In certain embodiments, Ab2 binds a tumor-associated antigen (TAA), preferably a human tumor-associated antigen (human TAA). The TAA can, for example, be DLL3.

In certain embodiments, the bispecific antibody or antigen-binding fragment thereof comprises: a first antigen-binding arm (Ab1) comprising H1 and L1 and a second antigen-binding arm (Ab2) comprising H2 and L2, wherein
(a) H1 and H2 each comprises a CH1 region of human IgG1, IgG2, IgG3, or IgG4; and
(b) L1 and L2 each comprises a CL region of a human kappa light chain or a human lambda light chain;
   wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) K/R133D/E in CH1 of H1 and K207K/R in CL of L1, respectively, and K/R133K/R in CH1 of H2 and K207D/E in CL of L2, respectively;
(6) K/R133D/E in CH1 of H1 and I/L117K/R in CL of L1, respectively, and K/R133K/R in CH1 of H2 and I/L117D/E in CL of L2, respectively;
(7) K/R133D/E in CH1 of H1 and F/V209K/R in CL of L1, respectively, and K/R133K/R in CH1 of H2 and F/V209D/E in CL of L2, respectively;
(8) G166D/E in CH1 of H2 and S114K/R in CL of L2, respectively, and G166K/R in CH1 of H1 and S114D/E in CL of L1, respectively;
(9) T187D/E in CH1 of H2 and D/N170K/R in CL of L2, respectively, and T187K/R in CH1 of H1 and D/N170D/E in CL of L1, respectively;
(10) S131D/E in CH1 of H2 and P119K/R in CL of L2, respectively, and S131K/R in CH1 of H1 and P119D/E in CL of L1, respectively;
(11) A129D/E in CH1 of H2 and S121K/R in CL of L2, respectively, and A129K/R in CH1 of H1 and S121D/E in CL of L1, respectively;
(12) K/R133D/E in CH1 of H2 and K207K/R in CL of L2, respectively, and K/R133K/R in CH1 of H1 and K207D/E in CL of L1, respectively;
(13) K/R133D/E in CH1 of H2 and I/L117K/R in CL of L2, respectively, and K/R133K/R in CH1 of H1 and I/L117D/E in CL of L1, respectively; or
(14) K/R133D/E in CH1 of H2 and F/V209K/R in CL of L2, respectively, and K/R133K/R in CH1 of H1 and F/V209D/E in CL of L1, respectively.

In certain embodiments, the bispecific antibody or antigen-binding fragment thereof comprises: a first antigen-binding arm (Ab1) comprising a VH region having a polypeptide sequence of SEQ ID NO:15, a VL region having a polypeptide sequence of SEQ ID NO:17, a CH1 region having a polypeptide sequence of SEQ ID NO:16, and a CL region having a polypeptide sequence of SEQ ID NO:18.

In certain embodiments, the bispecific antibody or antigen-binding fragment thereof comprises: a first antigen-binding arm (Ab1) comprising a VH region having a polypeptide sequence of SEQ ID NO:19, a VL region having a polypeptide sequence of SEQ ID NO:21, a CH1 region having a polypeptide sequence of SEQ ID NO:20, and a CL region having a polypeptide sequence of SEQ ID NO:22.

In certain embodiments, the bispecific antibody or antigen-binding fragment thereof comprises: a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18.

In certain embodiments, the bispecific antibody or antigen-binding fragment thereof comprises: a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22.

In certain embodiments, the bispecific antibody or antigen-binding fragment thereof comprises:
(a) a first antigen-binding arm (Ab1) comprising a VH region having a polypeptide sequence of SEQ ID NO:15, a VL region having a polypeptide sequence of SEQ ID NO:17, a CH1 region having a polypeptide sequence of SEQ ID NO:16, and a CL region having a polypeptide sequence of SEQ ID NO:18; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26;
(b) a first antigen-binding arm (Ab1) comprising a VH region having a polypeptide sequence of SEQ ID NO:19, a VL region having a polypeptide sequence of SEQ ID NO:21, a CH1 region having a polypeptide sequence of SEQ ID NO:20, and a CL region having a polypeptide sequence of SEQ ID NO:22; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26;
(c) a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26; or
(d) a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26.

In certain embodiments, the isolated antibody or antigen-binding fragment thereof is conjugated to the FA at the substituted amino acid residue. The FA can, for example, be selected from a FA with 6 carbons, 8 carbons, 10 carbons, 12 carbons, 14 carbons, 16 carbons, or 18 carbons, or any number of carbons in between. In certain embodiments, the FA is selected from a FA with 14 carbons or 18 carbons or any number of carbons in between.

In certain embodiments, the FA comprises a linker for conjugation to the substituted amino acid residue. The linker can, for example, be selected from a peptide linker or a polyethylene glycol (PEG) linker. The peptide linker can, for example, be less than 50 amino acids.

In certain embodiments, the FA conjugated to the antibody or antigen-binding fragment thereof is capable of binding albumin, wherein the binding of albumin to the FA results in a partial or a complete blocking of the binding between the target antigen and the antibody or antigen-binding fragment thereof. In certain embodiments, wherein the isolated antibody or antigen-binding fragment thereof is a bispecific antibody or antigen-binding fragment thereof, the binding of albumin to the FA on the Ab1 arm does not affect the binding of the Ab2 arm to its antigen or the binding of albumin to the FA on the Ab2 arm does not affect the binding of the Ab1 arm to its antigen. In certain embodiments, the isolated antibody or antigen-binding fragment thereof conjugated to a FA has a reduced ability to activate T cells upon binding to albumin as compared to the isolated antibody or antigen-binding fragment thereof conjugated to the FA not binding to albumin.

Also provided are isolated nucleic acids encoding the isolated antibodies or antigen-binding fragments thereof of the invention.

Also provided are vectors comprising the isolated nucleic acids encoding the isolated antibodies or antigen-binding fragments thereof of the invention.

Also provided are host cells comprising the vectors of the invention.

Also provided are pharmaceutical compositions comprising an isolated antibody or antigen-binding fragment thereof of the invention and a pharmaceutically acceptable carrier. In certain embodiments, the pharmaceutical compositions comprise an isolated antibody or antigen-binding fragment thereof conjugated to a FA and a pharmaceutically acceptable carrier. In certain embodiments, the pharmaceutical compositions comprise an isolated antibody or antigen-binding fragment thereof conjugated to a FA, wherein the FA is bound to albumin, and a pharmaceutically acceptable carrier.

Also provided are methods of treating a cancer in a subject in need thereof, the methods comprising administering to the subject pharmaceutical compositions of the invention. The cancer can, for example, be selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

Also provided are methods of producing the isolated antibody or antigen-binding fragment thereof of the invention. The methods comprise culturing a cell comprising a nucleic acid encoding the antibody or antigen-binding fragment thereof under conditions to produce the antibody or antigen-binding fragment thereof, and, optionally, recovering the antibody or antigen-binding fragment thereof from the cell or culture.

Also provided are methods of producing the isolated antibody or antigen-binding fragment thereof conjugated to a FA of the invention. The methods comprise conjugating the FA to the antibody or antigen-binding fragment thereof at the substituted amino acid residue. Also provided are methods of producing the isolated antibody or antigen-binding fragment thereof conjugated to a FA and bound to an albumin, the methods comprise contacting an isolated antibody or antigen-binding fragment thereof conjugated to a FA with albumin.

Also provided are methods of producing a pharmaceutical composition comprising the isolated antibody or antigen-binding fragment thereof of the invention. The methods comprise combining the antibody or antigen-binding fragment thereof with a pharmaceutically acceptable carrier to obtain the pharmaceutical composition.

Also provided are methods comprising contacting albumin with a conjugate comprising a FA covalently linked, optionally through a linker, to an antibody or antigen-binding fragment thereof, wherein the antibody or antigen-binding fragment thereof in the conjugate is capable of specific binding to a target antigen, the FA in the conjugate is capable of binding to albumin, and the binding of albumin to the FA results in a partial or a complete blocking of the binding between the target antigen and the antibody or antigen-binding fragment thereof. In certain embodiments, the antibody or antigen-binding fragment thereof comprises one or more substituted amino acid residues in the VH, VL, or within a twenty (20)-amino acid distance, preferably a five (5)-amino acid distance, of the VH or VL, and the one or more substituted amino acid residues are covalently linked, optionally through the linker, to the FA(s). In certain embodiments, the contacting step comprises administering a pharmaceutical composition comprising the conjugate to a subject in need of a treatment of a tumor, wherein the tumor comprises the target antigen. In certain embodiments, albumin has a higher turnover rate in the tumor microenvironment compared with the circulating blood, and/or albumin is present in the tumor microenvironment at a level lower than the albumin level in the circulating blood of the subject.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1E show schematic structures of a monoclonal antibody (mAb) (FIG. 1A) and a bispecific antibody (bsAb) (FIGS. 1B and 1C) with a fatty acid (FA) molecule conjugated to the VH region, an illustration of the mode of action of a FA-conjugated bispecific antibody in vivo (FIG. 1D), and a schematic of a strategy to identify FA-conjugated antibodies (FIG. 1E). FIGS. 1A-1C provide schematics for illustration purposes only because the conjugation site can be on other suitable sites within the Fab region, including the VL region and the CL region. Additionally, both arms of the bispecific antibody can be conjugated in FIGS. 1B-1C. The FA can potentially modulate the antigen-binding activity of the conjugated arm in different degrees. In FIG. 1A, the FA molecule is shown to be conjugated to the VH region of both arms; in FIG. 1B, the FA molecule is shown to be conjugated to the VH region of one of the arms of the bispecific antibody; in FIG. 1C, the FA molecule is shown to be conjugated to the CH1 region of one of the arms of the bispecific antibody. The antigen-binding activity of the conjugated arm is expected to be modulated by albumin because the albumin bound to the conjugated FA can completely or partially block the binding of the target antigen by the conjugated arm. FIG. 1D illustrates the mode of action of an FA-conjugated bispecific antibody for use to engage T cells to target cancer cells in vivo. When both arms of the bispecific antibody are conjugated with FA, the same goal can be achieved; however, the modulation by albumin in this case can be increased compared with bispecific antibodies with only one arm being conjugated. Additionally, FIG. 1D illustrates how albumin level regulates the antigen-binding activity of a FA-conjugated bispecific antibody. FIG. 1E shows the specific steps for identifying a FA-conjugated mAb or bsAb. Albumin-dependent activity refers to the fact that the activity of the conjugated antibody is modulated by albumin (i.e., high concentrations of albumin reduce or completely blocks the antigen-binding activity).

FIGS. 2A-2D show the amino acid sequences of example antibodies. FIG. 2A shows the amino acid sequence of the VH region of an anti-CD3 antibody (SEQ ID NO:1). FIG. 2B shows the amino acid sequence of the VL region of an anti-CD3 antibody (SEQ ID NO:2). FIG. 2C shows the amino acid sequences of the CH1 regions of human IgG1 (SEQ ID NO:9), IgG2 (SEQ ID NO:10), IgG3 (SEQ ID NO:11), and IgG4 (SEQ ID NO:12). FIG. 2D shows the amino acid sequences of the CL regions of human kappa (SEQ ID NO:13) and lambda light chains (SEQ ID NO:14). The CDR regions determined by a combination of IMGT and Kabat methods are highlighted in grey. * represents sites of known allelic variations.

FIGS. 3A-3G show examples of selected amino acid residues for substitution and conjugation with a FA in an anti-CD3 monoclonal antibody (mAb). FIG. 3A shows the 3-D modeling of the Fab region (containing VH, CH1, VL, and CL) in an anti-CD3 mAb to identify potential sites for cysteine knock-in for FA conjugation. Four sites are shown in the 3-D structure as examples (LC_S26, LC_S31, HC_K64, and HC_T120) (LC: light chain; HC: heavy chain). Additional sites are shown in Table 3. FIGS. 3B-3G show graphs demonstrating the binding of the anti-CD3 mAbs with cysteine knock-ins to CD3 on Jurkat cells. MFI: median fluorescence intensity. Anti-CD3 mAb, the wildtype anti-CD3 mAb.

FIGS. 4A-4C show the structures of FA molecules for conjugation with the anti-CD3 mAb and the mass spectrometry (MS) profiles of the FA-conjugated anti-CD3 mAbs. FIG. 4A shows the structures of the FA molecules for conjugation. All the FA molecules were conjugated via a PEG linker. FIG. 4B shows the MS profiles of the mAbs (LC_S26C, HC_K64C, and HC_T120C) conjugated with the C18 FA. FIG. 4C shows the MS profiles of the HC_K64C mAb conjugated with the C6, C10, and C14 FA, respectively. Expt, expected deconvoluted mass; obs, observed deconvoluted mass. LC_S26C represents the anti-CD3 mAb where the serine at S26 position of the light chain is replaced with cysteine; all the other mAbs with a cysteine knocked in follow the same naming rule.

FIGS. 5A-5C show the effect of albumin on the binding of the C18 FA-conjugated anti-CD3 mAbs to CD3 on Jurkat cells. The assay was carried out in the absence or presence of 50 mg/mL bovine serum albumin (BSA). FIG. 5A, conjugated LC_S26C; FIG. 5B, conjugated HC_K64C; FIG. 5C, conjugated HC_T120C.

FIGS. 6A-6C show the effect of different concentrations of albumin on T cell activation by the C18 FA-conjugated anti-CD3 mAbs. FIG. 6A, conjugated LC_S26C; FIG. 6B, conjugated HC_K64C; FIG. 6C, conjugated HC_T120C. The assay media contains 1% FBS (fetal bovine serum); the labeled BSA concentration represents the BSA added to the assay media.

FIGS. 7A-7C show the effect of different concentrations of albumin on T cell activation by the C6, C10, and C14 FA-conjugated anti-CD3 mAbs, respectively. FIG. 7A, C6 FA-conjugated HC_K64C; FIG. 7B, C10 FA-conjugated HC_K64C; FIG. 7C, C14 FA-conjugated HC_K64C. The assay media contains 1% FBS; the labeled BSA concentration represents the BSA added to the assay media; Control, no BSA was added.

FIGS. 8A-8C show the purity of the purified anti-DLL3/anti-CD3 bispecific antibody bsAb HC_K64C where the residue K64 on the HC of the anti-CD3 arm is replaced with cysteine. FIG. 8A shows the result of HIC HPLC analysis of the purified bsAb HC_K64C with certain impurity standards; FIG. 8B shows the result of the SCX HPLC analysis of the purified bsAb HC_K64C with certain impurity standards; FIG. 8C shows the result of the SEC HPLC analysis of the purified bsAb HC_K64C. Anti-CD3 knob homodimer/half mol., impurity standard that was Protein A purified from the media of cells transfected with the anti-CD3 HC and anti-CD3 LC; anti-DLL3 hole homodimer/half mol., impurity standard that was Protein A purified from the media of cells transfected with the anti-DLL3 HC and anti-DLL3 LC; 2× anti-CD3 LC mismatch, impurity standard that was Protein A purified from the media of cells transfected with the anti-CD3 HC, anti-CD3 LC and anti-DLL3 HC; 2× anti-DLL3 LC mismatch, impurity standard that was Protein A purified from the media of cells transfected with the anti-CD3 HC, anti-DLL3 HC and anti-DLL3 LC. Half mol., half IgG molecule with only one HC and one LC.

FIGS. 9A-9C show the purity of the purified anti-DLL3/anti-CD3 bispecific antibody bsAb HC_T120C where the residue T120 on the HC of the anti-CD3 arm is replaced with cysteine. FIG. 9A shows the result of the HIC HPLC analysis of the purified bsAb HC_T120C with certain impurity standards; FIG. 9B shows the result of the SCX HPLC analysis of the purified bsAb HC_T120C with certain impurity standards; FIG. 9C shows the result of the SEC HPLC analysis of the purified bsAb HC_T120C. Anti-CD3 knob homodimer/half mol., impurity standard that was Protein A purified from the media of cells transfected with the anti-CD3 HC and anti-CD3 LC; anti-DLL3 hole homodimer/half mol., impurity standard that was Protein A purified from the media of cells transfected with the anti-DLL3 HC and anti-DLL3 LC; 2× anti-CD3 LC mismatch, impurity standard that was Protein A purified from the media of cells transfected with the anti-CD3 HC, anti-CD3 LC and anti-DLL3 HC; 2× anti-DLL3 LC mismatch, impurity standard that was Protein A purified from the media of cells transfected with the anti-CD3 HC, anti-DLL3 HC and anti-DLL3 LC. Half mol., half IgG molecule with only one HC and one LC.

FIGS. 10A-10B show the purity of the purified anti-DLL3/anti-CD3 bispecific antibodies conjugated with fatty acid molecules. FIG. 10A shows the result of the HIC HPLC analyses of the purified anti-DLL3/anti-CD3 bispecific antibodies conjugated with fatty acid molecules; FIG. 10B shows the result of the SEC HPLC analyses of the purified anti-DLL3/anti-CD3 bispecific antibodies conjugated with fatty acid molecules. bsAb HC_T120C_C18 refers to the anti-DLL3/anti-CD3 bispecific antibody bsAb HC_T120C conjugated with C18 FA; the other conjugated bispecific antibodies follow the same naming rule.

FIG. 11 shows the result of the crosslinking assay of SHP-77 and Jurkat cells by the unconjugated and conjugated anti-DLL3/anti-CD3 bispecific antibodies in the presence or absence of blocking antibodies. The anti-DLL3 blocking mAb is the mAb version of the anti-DLL3 arm; the anti-CD3 blocking mAb is the mAb version of the anti-CD3 arm (without knocked in cysteine). WT bsAb, the wildtype anti-DLL3/anti-CD3 bispecific antibody (no cysteine knock-in); SHP-77+ Jurkat control, the assay was done with the cells without added antibody.

FIGS. 12A-12B show the results for the activation of the T-cell-receptor-CD3 (TCR/CD3) complex on Jurkat cells in the presence of SHP-77 cells (expressing DLL3) mediated by the unconjugated and conjugated anti-DLL3/anti-CD3 bispecific antibodies. The anti-DLL3 blocking mAb was used to suppress the activation to demonstrate that the Jurkat cell activation requires the simultaneous binding of the SHP-77 cells by the bispecific antibodies. The assay media contains 0.5% FBS.

FIGS. 13A-13B show the results for the effect of BSA on the activation of the TCR/CD3 complex on Jurkat cells in the presence of SHP-77 cells (expressing DLL3; also known as the target cell) mediated by the unconjugated and conjugated anti-DLL3/anti-CD3 bispecific antibodies. The assay media contains 0.5% FBS; the labeled BSA concentration represents the BSA added to the assay media.

FIG. 14 shows the result of the ELISA assay used to assess the effect of BSA on the antigen-binding activity of the anti-DLL3 arm of the conjugated bispecific antibodies. Anti-DLL3 F(ab′)2 was used as control for inhibition of DLL3 binding by the anti-DLL3 arm of the conjugated bispecific antibodies.

### DETAILED DESCRIPTION OF THE INVENTION

Various publications, articles, and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the terms “different heavy chains” or “different light chains” as used throughout the specification and claims, indicate that the heavy chains or the light chains have sequences that are not identical to each other.

The terms “identical” or percent “identity,” in the context of two or more nucleic acids or polypeptide sequences (e.g., anti-DLL3 antibodies, anti-CD3 antibodies, anti-CD3/anti-DLL3 bispecific antibodies, DLL3 polypeptides and polynucleotides that encode them, and CD3 polypeptides and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed monoclonal or bispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As described herein, the term “CD3” refers to Cluster of Differentiation 3, which is a multi-subunit protein complex that functions as the co-receptor to T cell receptor (TCR) (Dong et al., Nature 573(7775):546-552 (2019)). Binding of TCR to peptide-MHC (pMHC) on the surface of the target cells induces the clustering of the TCR-CD3 complex and activates the intracellular signaling mediated by the ζ chain of CD3 (Annu Rev Immunol. 27:591-619 (2009)). CD3 is required for the activation of T-cells and its pMHC-independent activation by therapeutics, such as in CAR-T-cells and by CD3-based T cell engagers, is highly effective in mobilizing T cells to kill tumor cells (Brown and Mackall, Nat Rev Immunol 19(2):73-74 (2019) and Clynes and Desjarlais, Annu Rev Med 70:437-450 (2019)). An exemplary amino acid sequence of a human CD3 epsilon subunit is represented in GenBank Accession No. NP 000724.1.

As described herein, the term “DLL3” refers to Delta like canonical Notch ligand 3 (DLL3), also known as delta like 3 or delta like protein 3, which is required for somite segmentation during early development (Dunwoodie et al., Development 129:1795-806 (2002)). Unlike the mammalian Notch family ligands DLL1, DLL4, JAG1, and JAG2 which all activate Notch receptor signaling in trans (Ntziachristos et al., Cancer Cell 25(3):318-34 (2014)), DLL3 is predominantly localized in the Golgi apparatus and is unable to activate Notch signaling (Chapman et al., Hum Mol Genet 20(5):905-16 (2011) and Geffers et al., J Cell Biol 178(3):465-76 (2007)). During normal development, DLL3 inhibits both cis- and trans-acting Notch pathway activation by interacting with Notch and DLL1 (Chapman et al., Hum Mol Genet 20(5):905-16(2011)). DLL3 is normally either absent or present at very low levels in adult normal tissues except brain, but is overexpressed in lung cancer, testicular cancer, glioma and melanoma samples (Uhlen et al., Science 357(6352): eaan2507 (2017)). Furthermore, DLL3 is detectable on the surface of small cell lung cancer (SCLC) and large cell neuroendocrine carcinoma (LCNEC) tumor cells (Saunders et al., Sci Transl Med 7(302):302ra136 (2015) and Sharma et al., Cancer Res 77(14):3931-41 (2017)), making it a potential target of monoclonal antibodies for cancer therapy. Therefore, an anti-DLL3 monoclonal antibody could be used to specifically target DLL3-expressing tumor cells and serve as a potential anti-cancer therapeutic. The term “human DLL3” refers to a DLL3 originated from a human. An exemplary amino acid sequence of a human DLL3 is represented in GenBank Accession No. NP_058637.1.

A “tumor-associated antigen (TAA),” as described herein, refers to any cell surface peptide and/or antigen or a combination of a cell surface peptide and/or antigen and its post-translational modifying moiety (such as glycosylation) that are present at a higher level in tumor than in normal tissues. Some of the tumor-associated antigens present specifically in tumors are also known as tumor-specific antigens (TSAs). Examples of tumor-associated antigens are viral proteins encoded by oncogenic viruses; mutated oncoproteins or tumor suppressors; normal proteins overexpressed on and/or in tumor cells; post-translational modifications of cell surface proteins; oncofetal proteins, whose expression are normally restricted in development stages but not in adult tissues; and cell-type specific proteins, whose expression are limited to unessential tissues.

A “fatty acid” as described herein, refers to a chemical molecule comprised of hydrocarbon chains terminating with carboxylic acid groups generally with 6-22 carbon atoms. For the invention here, various fatty acid derivatives are also considered fatty acids for their ability to bind to albumin. Fatty acids and their derivatives are the primary components of lipids and confer hydrophobic properties. The length and degree of saturation of the hydrocarbon chain vary among fatty acids which determine the associated physical properties. Types of fatty acids include unsaturated fatty acids (polyunsaturated and monounsaturated) and saturated fatty acids; saturated fatty acids are saturated with hydrogen and are mostly straight hydrocarbon chains with an even number of carbon atoms.

An “immune cell modulator (ICM),” as described herein, refers to any cell surface molecule such as a protein that is expressed on the surface of immune cells and regulate the function of the immune cells. The ICMs include stimulatory molecules and inhibitory molecules. A stimulatory ICM can mediate the activation of the immune cells when a specific antibody or antigen-binding fragment with certain characteristics specifically binds to the stimulatory ICM. An inhibitory ICM suppresses the activity of the immune cell upon binding by a ligand/interacting partner, which can be blocked by a specific antibody or antigen-binding fragment with certain characteristics leading to the activation of the immune cells. These immune cells can be T cells, NK cells, macrophages or other types of cells of the immune system. Examples of ICMs include, but are not limited to, CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules.

As used herein the term “complete block” or “complete blockade” refers to the complete inhibition of a target antigen (e.g., an ICM, such as CD3) binding to the target antigen-binding domain (e.g., a monoclonal or bispecific antibody or antigen-binding fragment thereof). The complete inhibition of target antigen-binding means that there is no binding (e.g., 0% binding) of the target antigen to the target antigen-binding domain.

As used herein the term “partial block” or “partial blockade” refers to an incomplete inhibition of a target antigen (e.g., an ICM, such as CD3) binding to the target antigen-binding domain (e.g., a monoclonal or bispecific antibody or antigen-binding fragment thereof). The incomplete inhibition of target antigen-binding means that there is at least some binding (e.g., 1% to 99% binding) of the target antigen to the target antigen-binding domain.

As used herein the term “specific binding” refers to the significant binding of the target antigen to an antibody or antigen-binding fragment thereof as compared to a control antigen, and/or the significant binding of the target antigen to an antibody or antigen-binding fragment thereof as compared to a control antibody or antigen-binding fragment, wherein the control antigen is different from the target antigen by sequence and/or structure comparison, and the control antibody or antigen-binding fragment significantly and selectively binds only to its corresponding antigen that is different from the target antigen by sequence and/or structure comparison.

Antibodies

The invention generally relates to monoclonal antibodies (mAbs) (e.g., anti-ICM mAbs, such as anti-CD3 mAbs) or bispecific antibodies (bsAbs) (e.g., anti-CD3/anti-DLL3 bsAbs) comprising a fatty acid (FA) molecule conjugated to or near the antigen-binding domain comprising a variable heavy chain region (VH) and a variable light chain region (VL) (e.g., in the VH, the VL, or within a twenty (20)-amino acid distance, preferably a five (5)-amino acid distance, of either the VH or the VL). The conjugation site is a reactive residue in or near the antigen-binding domain and can be a knocked in cysteine or another reactive amino acid. The location of the conjugation site is identified such that the knocked in cysteine (or other reactive amino acid) or the conjugated FA does not eliminate the target antigen (e.g., the ICM, such as CD3) binding activity of the antigen-binding domain. The conjugated FA can bind to an albumin molecule, and the bound albumin molecule can occupy a significant space in between the antigen-binding domain and the target antigen (e.g., the ICM, such as CD3). The bound albumin molecule can sterically hinder the binding of the target antigen to the antigen-binding domain, leading to a reduction in or complete blocking of binding of the target antigen with the antigen-binding domain. The FA-conjugated mAb or the FA-conjugated arm of the bsAb can be against an immune cell modulator (ICM), which upon antibody binding, can lead to immune cell activation. Examples of ICMs include, but are not limited to, CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules. Thus, the immune cell activation activity of the conjugated mAb or bsAb can be regulated by albumin bound to the conjugated FA. The extent of the regulation depends on the concentration of albumin around the conjugated mAb or bsAb, the length of the FA molecule, and the specific location of the conjugation site. The invention also relates to multi-specific antibodies or antigen-binding fragments thereof, wherein the multi-specific antibody or antigen-binding fragment thereof comprises one or more antigen-binding arm(s) comprising a substituted amino acid residue that is conjugated to a FA.

The conjugated FA on the mAb or bsAb or multi-specific antibody can be bound by circulating albumin in the blood, which can serve to decrease or block the binding of the conjugated mAb or bsAb to the target antigen on T cells (e.g., an ICM, such as CD3), leading to partial or complete inhibition of T cell activation. In the tumor microenvironment, where there is a higher albumin turnover rate compared with the circulating blood, and the local albumin level is expected to be lower than in the circulating blood, the conjugated antibodies have less or no albumin bound to them, which can lead to increased target antigen-binding (e.g., CD3) and T cell activation. Additionally, or alternatively, the higher albumin turnover rate in the tumor microenvironment can reduce the level of albumin-bound mAb or bsAb or multi-specific antibody and expose the antigen-binding domain, leading to increased target antigen-binding (e.g., CD3) and T cell activation. The conjugated antibodies can have advantages with respect to safety in vivo and can be used for therapeutic purposes. The conjugated bsAbs can also be used as T cell engagers or other immune cell engagers where one arm comprises an antigen-binding domain against a tumor-associated antigen (TAA) and the other arm comprises a conjugated anti-ICM antigen-binding region (e.g., an anti-CD3 antigen-binding region).

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3, and IgG4. Accordingly, the antibodies of the invention can be of any of the five major classes or corresponding sub-classes. Preferably, the antibodies of the invention are IgG1, IgG2, IgG3, or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

Several systems are used for the numbering of amino acid residues in antibodies. The Kabat numbering method is a scheme based on variable regions of antibodies (Elvin A. Kabat et al., Sequences of Proteins of Immunological Interest 5th ed. (1991). The EU numbering system is widely used for the constant domains (including portions of the CH1, hinge, and the Fc) (Elvin A. Kabat et al., Sequences of Proteins of Immunological Interest 5th ed. (1991).

As used herein, the term an “isolated antibody” refers to an antibody which is substantially free of other antibodies having different antigenic specificities (e.g., an isolated antibody that specifically binds to DLL3 is substantially free of antibodies that do not bind to DLL3; an isolated antibody that specifically binds to CD3 is substantially free of antibodies that do not bind to CD3; a bispecific antibody that specifically binds to CD3 and DLL3 is substantially free of antibodies that do not bind to CD3 and DLL3). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, a Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab), a scFv dimer (bivalent diabody), a multi-specific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and a Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multi-specific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multi-specific antibody comprises a third, fourth, or fifth immunoglobulin variable domain. In an embodiment, a multi-specific antibody is a bispecific antibody molecule, a trispecific antibody molecule, or a tetraspecific antibody molecule.

As used herein, the term “bispecific antibody” refers to a multi-specific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope.

As used herein, the term “CD3” refers to cluster of differentiation 3. An exemplary amino acid sequence of a human CD3 epsilon subunit is represented in GenBank Accession No. NP_000724.1. The term “4-1BB” refers to tumor necrosis factor receptor superfamily member 9 (TNFRSF9), also known as CD137 and ILA (induced by lymphocyte activation). An exemplary amino acid sequence of a human 4-1BB is represented in GenBank Accession No. NP_001552.2. The term “OX40” refers to tumor necrosis factor receptor superfamily member 4 (TNFRSF4), also known as CD134. An exemplary amino acid sequence of a human OX40 is represented in GenBank Accession No. NP_003318.1. The term “CD28” refers to cluster of differentiation 28. Exemplary amino acid sequences of human CD28 variants are represented in GenBank Accession Nos. NP 001230006.1, NP 001230007.1, NP 006130.1, XP 011510496.1, and XP 011510499.1. The term “PD-1” refers to programmed cell death 1. An exemplary amino acid sequence of a human PD-1 is represented in GenBank Accession No. NP_005009.2. The term “GITR” refers to glucocorticoid-induced TNFR-related protein (GITR), also known as tumor necrosis factor receptor superfamily member 18 (TNFRSF18) or activation-inducible TNFR family receptor (AITR). Exemplary amino acid sequences of human GITR variants are represented in GenBank Accession Nos. NP 004186.1, NP 683699.1, and NP 683700.1. The term “VISTA” refers to V-domain Ig suppressor of T cell activation, also known as V-set immunoregulatory receptor (VSIR). An exemplary amino acid sequence of a human VISTA is represented in GenBank Accession No. NP_071436.1.

As used herein, an antibody that “specifically binds to CD3 and/or DLL3” refers to an antibody that binds to CD3 and/or DLL3, preferably a human CD3 and/or human DLL3, with a KD of 1×10⁻⁷ M or less, preferably 1×10⁻⁸ M or less, more preferably 5×10⁻⁹ M or less, 1×10⁻⁹ M or less, 5×10⁻¹° M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

According to one particular aspect, the invention relates to an isolated monoclonal antibody or antigen-binding fragment, wherein the antibody or antigen-binding fragment thereof comprises (a) a heavy chain variable region (VH); and a light chain variable region (VL); wherein the antibody or antigen-binding fragment thereof binds to a target antigen, preferably a human target antigen; wherein an amino acid residue in the VH, VL, or within a twenty (20)-amino acid distance, preferably a five (5)-amino acid distance, of the VH or VL is substituted with an amino acid residue that is conjugated to a fatty acid (FA); and wherein upon conjugation with the FA at the substituted amino acid residue, the antibody or antigen-binding fragment still binds to the target antigen; and wherein the FA-conjugated antibody or antigen-binding fragment thereof has reduced or eliminated specific binding to the target antigen in the presence of physiological levels of albumin (e.g., 35 to 50 mg/mL). The substituted amino acid residue can, for example, be a cysteine residue or a lysine residue.

As used herein, the phrase “within a twenty (20)-amino acid distance of the VH or VL” refers to a residue within the CH1 or CL region that is less than 20-amino acid distance from the variable heavy or light chain. The phrase “within a five (5)-amino acid distance of the VH or VL” refers to a residue within the CH1 or CL region that is less than 5-amino acid distance from the variable heavy or light chain.

As used herein, the phrase “still binds to the target antigen” indicates that the antibody or antigen-binding fragment thereof, when conjugated to the fatty acid (FA), is still capable of binding the target antigen. The level of binding of the target antigen to the FA-conjugated antibody or antigen-binding fragment thereof can, for example, be about 10% to about 100% of the level of binding of the target antigen to the antibody or antigen-binding fragment thereof comprising an amino acid substitution for conjugation of the invention in the absence of the conjugated FA. In certain embodiments, the level of binding of the target antigen to the FA-conjugated antibody or antigen-binding fragment thereof is about 10%, about 20%, about 30%, about 40%, about 50%, about 60%, about 70%, about 80%, about 90%, or about 100% of the level of binding of the target antigen to an antibody or antigen-binding fragment thereof comprising an amino acid substitution for conjugation of the invention in the absence of the conjugated FA. A person skilled in the art would be able to determine the level of binding of a FA-conjugated antibody or antigen-binding fragment thereof to the target antigen utilizing methods known in the art. The level of binding can be compared to an antibody or antigen-binding fragment thereof comprising an amino acid substitution for conjugation of the invention, which is not conjugated to the fatty acid. The level of binding of the target antigen to the antibody or antigen-binding fragment thereof comprising an amino acid substitution for conjugation of the invention, which is not conjugated to the fatty acid, is at least 50% of that by the wildtype antibody or antigen-binding fragment.

According to a particular aspect, the substituted amino acid occurs at an amino acid residue corresponding to residue 25, 27, 62, 64, 73, 76, 101, 112, or 113 of SEQ ID NO:1 or an amino acid residue corresponding to residue 26, 27, 52, 53, 56, or 67 of SEQ ID NO:2, preferably the substitution is selected from a substitution corresponding to S25C, Y27C, K62C, K64C, K73C, S76C, D101C, S112C, or S113C of SEQ ID NO:1 or a substitution corresponding to S26C, S27C, S52C, K53C, S56C, or S67C of SEQ ID NO:2, wherein the residues are numbered according to Kabat. In certain embodiments, the substituted amino acid is at residue 64 corresponding to SEQ ID NO:1 or residue 26 corresponding to SEQ ID NO:2, preferably the substitution is selected from a K64C substitution corresponding to SEQ ID NO:1 or a S26C substitution corresponding to SEQ ID NO:2, wherein the residues are numbered according to Kabat.

As used herein, when referring to a substituted amino acid corresponding to an amino acid residue number of a SEQ ID NO, the SEQ ID NO is the reference for determining the substituted amino acid residue of the sequence of interest. A person skilled in the art would align the sequence of interest with the reference SEQ ID NO to determine the position of the amino acid residue to be substituted. By way of an example, amino acid residue number 25 of SEQ ID NO:1, which is the variable heavy chain region of an anti-CD3 monoclonal antibody, is a serine residue. Upon alignment with the variable heavy chain region of an antibody of interest, the residue that aligns with the serine residue at position number 25 of SEQ ID NO:1 would be targeted for an amino substitution.

According to a particular aspect, the substituted amino acid occurs at residue 119 or 120 in the CH1 of SEQ ID NO:9, 10, 11, or 12, or residue 121 or 124 in the CL of SEQ ID NO:13 or 14, preferably the substitution is selected from a S119C or T120C in the CH1 of SEQ ID NO:9, 10, 11, or 12, or a S121C or Q124C in the CL of SEQ ID NO:13 or 14, wherein the residues are numbered according to EU numbering. In certain embodiments, the substituted amino acid is at residue 120 in the CH1 region of SEQ ID NO:9, 10, 11, or 12, preferably the substitution is a T120C substitution in the CH1 region of SEQ ID NO:9, 10, 11, or 12, wherein the residues are numbered according to the EU numbering.

According to a particular aspect, the isolated antibody or antigen-binding fragment thereof is an anti-CD3 antibody or antigen-binding fragment thereof and is capable of specific binding to CD3, preferably human CD3. The isolated anti-CD3 antibody or antigen-binding fragment thereof can, for example, comprise a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:3, 4, 5, 6, 7, and 8, respectively, or SEQ ID NOs:33, 34, 35, 36, 37, and 38, respectively.

According to a particular aspect, the substituted amino acid is selected from residue 25, 27, 62, 64, 73, 76, 101, 112, or 113 in the VH of the anti-CD3 mAb (SEQ ID NO:1 or SEQ ID NO:27) or 26, 27, 52, 53, 56, or 67 in the VL of the anti-CD3 mAb (SEQ ID NO:2 or SEQ ID NO:28), preferably the substitution is selected from a S25C, Y27C, K62C, K64C, K73C, S76C, D101C, S112C, or S113C in the VH (SEQ ID NO:1 or 27) or a S26C, S27C, S52C, K53C, S56C, or S67C in the VL (SEQ ID NO:2 or 28), wherein the residues are numbered according to Kabat. In certain embodiments, the substituted amino acid is at residue 64 in the VH (SEQ ID NO:1 or 27) or 26 in the VL (SEQ ID NO:2 or 28), preferably the substitution is selected from a K64C substitution in the VH (SEQ ID NO:1 or 27) or a S26C substitution in the VL (SEQ ID NO:2 or 28), wherein the residues are numbered according to Kabat.

According to a particular aspect, the isolated anti-CD3 antibody or antigen-binding fragment thereof can, for example, comprise a VH region having a polypeptide sequence of SEQ ID NO:1 with an amino acid substitution of K64C and a VL region having a polypeptide sequence of SEQ ID NO:2; or a VH region having a polypeptide sequence of SEQ ID NO:27 with an amino acid substitution of K64C and a VL region having a polypeptide sequence of SEQ ID NO:28; or a VH region having a polypeptide sequence of SEQ ID NO:1 and a VL region having a polypeptide sequence of SEQ ID NO:2 with an amino acid substitution of S26C; or a VH region having a polypeptide sequence of SEQ ID NO:27 and a VL region having a polypeptide sequence of SEQ ID NO:28 with an amino acid substitution of S26C; or a CH1 region having a polypeptide sequence selected from SEQ ID NO: 9, 10, 11 or 12 with an amino acid substitution of T120C and a CL region having a polypeptide sequence selected from SEQ ID NO:13 or 14; or a VH region having a polypeptide sequence of SEQ ID NO:1, a VL region having a polypeptide sequence of SEQ ID NO:2, a CH1 region having a polypeptide sequence selected from SEQ ID NO: 9, 10, 11 or 12 with an amino acid substitution of T120C, and a CL region having a polypeptide sequence selected from SEQ ID NO:13 or 14; or a VH region having a polypeptide sequence of SEQ ID NO:27, a VL region having a polypeptide sequence of SEQ ID NO:28, a CH1 region having a polypeptide sequence selected from SEQ ID NO: 9, 10, 11 or 12 with an amino acid substitution of T120C, and a CL region having a polypeptide sequence selected from SEQ ID NO:13 or 14.

According to a particular aspect, provided herein is a multi-specific antibody or antigen-binding fragment thereof, wherein the multi-specific antibody or antigen-binding fragment thereof comprises a monoclonal antibody or antigen-binding fragment thereof of the invention, and wherein the multi-specific antibody or antigen-binding fragment thereof comprises one or more antigen-binding arm(s) comprising a substituted amino acid residue that is conjugated to a FA. The multi-specific antibody or antigen-binding fragment thereof can, for example, be a bispecific antibody or antigen-binding fragment thereof.

In certain embodiments, each arm of the multi-specific antibody or antigen-binding fragment thereof can contain a substituted amino acid at a different residue position with the same or different conjugated FA. In certain embodiments, each arm of the multi-specific antibody or antigen-binding fragment thereof can contain the same substituted amino acid at a different residue position with the same or different conjugated FA. In certain embodiments, each arm of the multi-specific antibody or antigen-binding fragment thereof can comprise a substituted amino acid at the same residue position with the same or different conjugated FA. In certain embodiments, each arm of the multi-specific antibody or antigen-binding fragment thereof can comprise the same substituted amino acid at the same residue position with the same or different conjugated FA.

In certain embodiments, the bispecific antibody or antigen-binding fragment thereof comprises a first antigen-binding arm (Ab1) and a second antigen-binding arm (Ab2), wherein Ab1 and/or Ab2 comprises a substituted amino acid that is conjugated to a FA.

In certain embodiments, Ab1 binds an immune cell modulator (ICM), preferably a human ICM, selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules. The ICM can, for example, be CD3, preferably human CD3.

In certain embodiments, Ab2 binds a tumor-associated antigen (TAA), preferably a human tumor-associated antigen (human TAA). The TAA can, for example, be DLL3.

In one embodiment of the invention, the isolated bispecific antibody or antigen-binding fragment thereof of the invention is an anti-CD3/anti-DLL3 bispecific antibody or antigen-binding fragment thereof, wherein the first antigen-binding arm (Ab1) specifically binds CD3, preferably human CD3, and the second antigen-binding arm (Ab2) specifically binds DLL3, preferably human DLL3.

According to a particular aspect, the bispecific antibody or antigen-binding fragment thereof comprises: a first antigen-binding arm (Ab1) comprising H1 and L1 and a second antigen-binding arm (Ab2) comprising H2 and L2, wherein
(a) H1 and H2 each comprises a CH1 region of human IgG1, IgG2, IgG3, or IgG4; and
(b) L1 and L2 each comprises a CL region of a human kappa light chain or a human lambda light chain;
   wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) K/R133D/E in CH1 of H1 and K207K/R in CL of L1, respectively, and K/R133K/R in CH1 of H2 and K207D/E in CL of L2, respectively;
(6) K/R133D/E in CH1 of H1 and I/L117K/R in CL of L1, respectively, and K/R133K/R in CH1 of H2 and I/L117D/E in CL of L2, respectively;
(7) K/R133D/E in CH1 of H1 and F/V209K/R in CL of L1, respectively, and K/R133K/R in CH1 of H2 and F/V209D/E in CL of L2, respectively;
(8) G166D/E in CH1 of H2 and S114K/R in CL of L2, respectively, and G166K/R in CH1 of H1 and S114D/E in CL of L1, respectively;
(9) T187D/E in CH1 of H2 and D/N170K/R in CL of L2, respectively, and T187K/R in CH1 of H1 and D/N170D/E in CL of L1, respectively;
(10) S131D/E in CH1 of H2 and P119K/R in CL of L2, respectively, and S131K/R in CH1 of H1 and P119D/E in CL of L1, respectively;
(11) A129D/E in CH1 of H2 and S121K/R in CL of L2, respectively, and A129K/R in CH1 of H1 and S121D/E in CL of L1, respectively;
(12) K/R133D/E in CH1 of H2 and K207K/R in CL of L2, respectively, and K/R133K/R in CH1 of H1 and K207D/E in CL of L1, respectively;
(13) K/R133D/E in CH1 of H2 and I/L117K/R in CL of L2, respectively, and K/R133K/R in CH1 of H1 and I/L117D/E in CL of L1, respectively; or
(14) K/R133D/E in CH1 of H2 and F/V209K/R in CL of L2, respectively, and K/R133K/R in CH1 of H1 and F/V209D/E in CL of L1, respectively.

As used herein, the term “charge pair” refers to a pair of amino acids with one having positive charge and the other having negative charge, which can be introduced by replacing native amino acid residues in the heavy chain CH1 region and the light chain CL region of the first arm of a bispecific antibody, respectively, and concurrently, the same pair of positive charge and negative charge amino acids can be introduced by replacing native amino acid residues in the light chain CL region and the heavy chain CH1 region of the second arm of the bispecific antibody, respectively. Alternatively, the positive charge and negative charge amino acids can be introduced by amino acid substitution to the VH region of the heavy chain and the VL region of the light chain of the first arm of a bispecific antibody, respectively, and concurrently, the same pair of positive charge and the negative charge amino acids can be introduced by amino acid substitution to the VL region of the light chain and the VH region of the heavy chain of the second arm, respectively. Amino acids used to form charge pairs usually include D/E (negative charge) and K/R (positive charge). Once introduced to the CH1/CL regions or VH/VL regions, the charge pair amino acids are in close proximity structurally and expected to enhance the heavy chain/light chain interaction of the same arm through opposite charges and expel the mismatched heavy chain/light chain interaction (the mismatched heavy and light chains are from the two different arms) through same charges. The resulting charge distribution of the introduced charge pair is as follows: H1 (CH1 positive charge)/L1 (CL negative charge)/H2 (CH1 negative charge)/L2 (CL positive charge) or H1 (CH1 negative charge)/L1 (CL positive charge)/H2 (CH1 positive charge)/L2 (CL negative charge). Multiple charge pairs can be combined and introduced to the CH1 and CL interface, with all positive charge amino acids introduced to CH1 and all negative charge amino acids to CL of the same arm, or vice versa, to satisfy the above distribution pattern. A similar approach can be applied to the VH/VL interface. Further, one or multiple charge pairs can also be introduced to the interface of VH and VL in combination with one or multiple charge pairs introduced to the CH1/CL interface—amino acids introduced to the same chain (either H1, L1, H2 or L2) usually have the same charge, and the resulting distribution of the introduced charge pairs is as follows: H1 (CH1 and VH positive charge)/L1 (CL and VL negative charge)/H2 (CH1 and VH negative charge)/L2 (CL and VL positive charge) or H1 (CH1 and VH negative charge)/L1 (CL and VL positive charge)/H2 (CH1 and VH positive charge)/L2 (CL and VL negative charge). The charge pair substitutions can also be combined with other modifications to further improve the cognate chain pairing preference (H1L1 and H2L2, respectively) and/or facilitate purification of the bispecific antibody using ion exchange chromatography and/or HIC. For example, in addition to the charge pair substitutions, the native interchain disulfide bond on one arm of the bispecific antibody can be shifted while the other arm has the native interchain disulfide bond (see, e.g., PCT/US2020/063066, filed on Dec. 3, 2020, which is incorporated by reference herein in its entirety).

In describing the charge pairs, G166D/E represents substitution of G at position 166 (EU numbering) with D or E, in which case G166 is the knock-in site; D170D/E represents keeping D at position 170 or substitution of D at position 170 with E; K/R133D/E represents substitution of K or R (whichever is at this position) at position 133 with D or E; all the other substitutions follow the same naming rule.

According to a particular aspect, the bispecific antibody or antigen-binding fragment thereof comprises: a first antigen-binding arm (Ab1) comprising a VH region having a polypeptide sequence of SEQ ID NO:15, a VL region having a polypeptide sequence of SEQ ID NO:17, a CH1 region having a polypeptide sequence of SEQ ID NO:16, and a CL region having a polypeptide sequence of SEQ ID NO:18.

According to a particular aspect, the bispecific antibody or antigen-binding fragment thereof comprises: a first antigen-binding arm (Ab1) comprising a first antigen-binding arm (Ab1) comprising a VH region having a polypeptide sequence of SEQ ID NO:19, a VL region having a polypeptide sequence of SEQ ID NO:21, a CH1 region having a polypeptide sequence of SEQ ID NO:20, and a CL region having a polypeptide sequence of SEQ ID NO:22.

According to a particular aspect, the bispecific antibody or antigen-binding fragment thereof comprises: a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18.

According to a particular aspect, the bispecific antibody or antigen-binding fragment thereof comprises: a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22.

According to a particular aspect, the bispecific antibody or antigen-binding fragment thereof comprises:
(a) a first antigen-binding arm (Ab1) comprising a VH region having a polypeptide sequence of SEQ ID NO:15, a VL region having a polypeptide sequence of SEQ ID NO:17, a CH1 region having a polypeptide sequence of SEQ ID NO:16, and a CL region having a polypeptide sequence of SEQ ID NO:18; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26;
(b) a first antigen-binding arm (Ab1) comprising a VH region having a polypeptide sequence of SEQ ID NO:19, a VL region having a polypeptide sequence of SEQ ID NO:21, a CH1 region having a polypeptide sequence of SEQ ID NO:20, and a CL region having a polypeptide sequence of SEQ ID NO:22; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26;
(c) a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26; or
(d) a first antigen-binding arm (Ab1) comprising a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22; and a second antigen-binding arm (Ab2) comprising a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26.

In certain embodiments, the isolated antibody or antigen-binding fragment thereof is conjugated to the FA at the substituted amino acid residue. The FA can, for example, be selected from a FA with 6 carbons, 7 carbons, 8 carbons, 9 carbons, 10 carbons, 11 carbons, 12 carbons, 13 carbons, 14 carbons, 15 carbons, 16 carbons, 17 carbons, or 18 carbons. In certain embodiments, the FA is selected from a FA with 14 carbons or 18 carbons or any number of carbons in between. The length of FA can determine the relative binding of albumin to the FA, which can determine the relative binding of the antibody or antigen-binding fragment thereof to the target antigen. The longer the conjugated FA is, the greater the binding affinity the conjugated FA has for albumin, leading to the greater albumin-mediated reduction of the specific binding to the target antigen by the conjugated mAb or bsAb. The shorter the FA is, the lower the binding affinity the conjugated FA has for albumin, leading to less or negligible albumin-mediated reduction of the specific binding to the target antigen by the conjugated mAb or bsAb.

In certain embodiments, the FA comprises a linker for conjugation to the substituted amino acid residue. The linker can, for example, be selected from a peptide linker or a polyethylene glycol (PEG) linker. The peptide linker can, for example, be less than 50 amino acids. The peptide linker can be 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 or less amino acids.

In certain embodiments, the FA conjugated to the antibody or antigen-binding fragment thereof is capable of binding albumin. The binding of albumin to the FA results in a partial or a complete blocking of the binding between the target antigen and the antibody or antigen-binding fragment thereof. In certain embodiments, wherein the isolated antibody or antigen-binding fragment thereof is a bispecific antibody or antigen-binding fragment thereof, wherein only the Ab1 arm is conjugated with a FA, the binding of albumin to the FA does not affect the binding of the Ab2 arm to the TAA. In certain embodiments, wherein the isolated antibody or antigen-binding fragment thereof is a bispecific antibody or antigen-binding fragment thereof, wherein both arms Ab1 and Ab2 are conjugated with FAs, the binding of albumin to the FAs results in the reduction or elimination of Ab1 and Ab2 binding to the target antigen for Ab1 and Ab2, respectively. In certain embodiments, the isolated antibody or antigen-binding fragment thereof has reduced ability to activate T cells upon binding to albumin as compared to the isolated antibody or antigen-binding fragment thereof not binding to albumin.

In one embodiment of the invention, the anti-CD3/anti-DLL3 bispecific antibody or antigen-binding fragment thereof of the invention is capable of activating T cells.

Full length bispecific antibodies of the invention can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e. an epitope on CD3 and an epitope on DLL3.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V_K409F_Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849.

In addition to methods described above, bispecific antibodies of the invention can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

Full length bispecific antibodies of the invention can be generated using a combination of the heterodimerization approaches above and several approaches as follows: (a) shifting the HC/LC interchain disulfide bond on one arm of the bispecific antibody (see, e.g., PCT/US2020/063066, filed on Dec. 3, 2020, which is incorporated by reference herein in its entirety); (b) introducing charge pairs to the VH/VL interface; (c) introducing charge pairs to the CH1/CL interface; or (d) a combination of some or all the approaches described in (a)-(c) (see, e.g., as first described in U.S. Provisional Patent Application No. 63/146,334, filed on Feb. 5, 2021, which is incorporated by reference herein in its entirety).

In another general aspect, the invention relates to an isolated nucleic acid encoding an isolated monoclonal antibody or antigen-binding fragment, or an isolated bispecific antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antibodies or antigen-binding fragments thereof of the invention can be altered without changing the amino acid sequences of the proteins.

In another general aspect, the invention relates to a vector comprising an isolated nucleic acid encoding an isolated monoclonal antibody or antigen-binding fragment, or an isolated bispecific antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention. Such techniques are well known to those skilled in the art in view of the present disclosure.

In another general aspect, the invention relates to a host cell comprising a vector comprising an isolated nucleic acid encoding an isolated monoclonal antibody or antigen-binding fragment, or an isolated bispecific antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., a scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing an isolated monoclonal antibody or antigen-binding fragment, or an isolated bispecific antibody or antigen-binding fragment thereof of the invention, comprising culturing a cell comprising a nucleic acid encoding the antibody or antigen-binding fragment thereof under conditions to produce an antibody or antigen-binding fragment thereof of the invention and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

In another general aspect, the invention relates to a method of producing the isolated antibody or antigen-binding fragment thereof conjugated to a FA of the invention. The methods comprise conjugating the FA to the antibody or antigen-binding fragment thereof at the substituted amino acid residue and recovering the antibody or antigen-binding fragment thereof conjugated to the FA.

In another general aspect, the invention relates to a method of producing the isolated antibody or antigen-binding fragment thereof conjugated to a FA and bound to an albumin. The methods comprise contacting an isolated antibody or antigen-binding fragment thereof conjugated to a FA with albumin and recovering the antibody or antigen-binding fragment thereof conjugated to the FA bound to albumin.

Pharmaceutical Compositions

In another general aspect, the invention relates to a pharmaceutical composition, comprising an isolated monoclonal antibody or antigen-binding fragment, or an isolated bispecific antibody or antigen-binding fragment thereof of the invention and a pharmaceutically acceptable carrier. The isolated monoclonal or bispecific antibody or antigen-binding fragment thereof can, for example, be conjugated to a fatty acid (FA). The FA-conjugated monoclonal or bispecific antibody or antigen-binding fragment thereof can, for example, be bound to albumin. The term “pharmaceutical composition” as used herein means a product comprising an antibody of the invention together with a pharmaceutically acceptable carrier. Antibodies of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include buffers, diluents, solvents, tonicity regulating agents, preservatives, stabilizers, and chelating agents. One or more pharmaceutically acceptable carriers can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of the isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propylene glycol), 1,3-propanediol, and 1,3-butanediol), polyethylene glycol (e.g. PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (e.g., PEG 3350), polyvinyl alcohol (PVA), polyvinyl pyrrolidone, salts (e.g., sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising an isolated monoclonal antibody or antigen-binding fragment, or an isolated bispecific antibody or antigen-binding fragment thereof of the invention, comprising combining an antibody or antigen-binding fragment thereof with a pharmaceutically acceptable carrier to obtain the pharmaceutical composition.

Methods of Use

In another general aspect, the invention relates to a method of targeting a tumor-associated antigen (TAA) (e.g., DLL3) expressed on a cancer cell surface in a subject in need thereof. The methods comprise administering to the subject a pharmaceutical composition comprising an isolated bispecific antibody or antigen-binding fragment thereof comprising an Ab1 arm (e.g., an anti-ICM arm, such as an anti-CD3 arm) conjugated to a FA (e.g., an anti-CD3/anti-DLL3 bispecific antibody or antigen-binding fragment thereof) of the invention and a pharmaceutically acceptable carrier. Binding of the isolated FA-conjugated bispecific antibody or antigen-binding fragment thereof to a TAA-expressing cancer cell via the anti-TAA arm (the Ab2 arm) and a T cell via the anti-CD3 arm (the Ab1 arm) simultaneously, at low albumin levels, can mediate cancer cell killing. In the circulating blood, where the albumin level is high (e.g., 35 to 50 mg/mL), the FA-conjugated anti-CD3 arm is in the albumin bound state, and, therefore, has reduced ability to bind to and activate a T cell. The T cell target antigen to which the Ab1 arm binds can be another T cell ICM, such as 4-1BB, GITR, CD28 or PD-1. This approach can increase the safety margin of an anti-ICM (e.g., an anti-CD3) based bispecific T cell engager by minimizing on-target, off-tumor toxicities. Further, the approach can be applied to bsAbs (comprising an anti-TAA arm and a conjugated anti-ICM arm) that can be used as engagers of other immune cells. In addition, the approach can be applied to using FA-conjugated mAbs and/or bsAbs to target tissues (such as adipose tissue or skeletal muscle) where the local albumin level is lower than that in the circulating blood to minimize on-target safety issues in the circulation (Ellmerer et al., Am J Physiol Endocrinol Metab. 2000. 278: E352-E356).

The functional activity of monoclonal antibodies or antigen-binding fragments thereof that bind a target antigen (e.g., an ICM, such as CD3), or bispecific antibodies and antigen-binding fragments thereof that bind both a TAA (e.g., DLL3) and a T cell target antigen (e.g., an ICM, such as CD3) can be characterized by methods known in the art and as described herein. Methods for characterizing bispecific antibodies and antigen-binding fragments thereof that bind both a TAA (e.g., DLL3) and a T cell target antigen (e.g., CD3) include, but are not limited to, affinity and specificity assays including Biacore, ELISA, FACS and OctetRed analysis. According to particular embodiments, the methods for characterizing bispecific antibodies and antigen-binding fragments thereof that bind both DLL3 and CD3 include those described below. The functional activity of monoclonal antibodies or antigen-binding fragments thereof that bind an ICM, or bispecific antibodies and antigen-binding fragments thereof that bind both a TAA (e.g., DLL3) and an ICM other than CD3 can be characterized by methods similar to those above.

In another general aspect, the invention relates to a method of treating a cancer in a subject in need thereof, comprising administering to the subject in need thereof an isolated monoclonal antibody or antigen-binding fragment, or an isolated bispecific antibody or antigen-binding fragment thereof or a pharmaceutical composition of the invention. The cancer can be any liquid or solid cancer, for example, it can be selected from, but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical composition comprises a therapeutically effective amount of a monoclonal antibody or antigen-binding fragment, or a bispecific antibody or antigen-binding fragment thereof of the invention. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to monoclonal and/or bispecific antibodies or antigen-binding fragments thereof, a therapeutically effective amount means an amount of the monoclonal and/or bispecific antibody or antigen-binding fragment thereof that modulates an immune response in a subject in need thereof. Also as used herein with reference to monoclonal and/or bispecific antibodies or antigen-binding fragments thereof, a therapeutically effective amount means an amount of the monoclonal and/or bispecific antibody or antigen-binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors. According to other particular embodiments, the disease, disorder or condition to be treated is an inflammatory disease, a metabolic disease, a cardiovascular disease, a neurological disease, an infectious disease, or any other disease where a bispecific antibody can be used as a therapy.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the compositions can be used in combination with another treatment including, but not limited to, a chemotherapy, an anti-TIM-3 mAb, an anti-LAG-3 mAb, an anti-CD73 mAb, an-anti-CD47 mAb, an anti-apelin mAb, an anti-CTLA-4 antibody, an anti-EGFR mAb, an anti-HER-2 mAb, an anti-CD19 mAb, an anti-CD20 mAb, an anti-CD33 mAb, an anti-TIP-1 mAb, an anti-DLL3 mAb, an anti-CLDN18.2 mAb, an anti-PD-L1 antibody, an anti-PD-1 antibody, a PD-1/PD-L1 therapy, other immuno-oncology drugs, an antiangiogenic agent, a radiation therapy, an antibody-drug conjugate (ADC), a targeted therapy, or other anticancer drugs.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Also provided are methods comprising contacting albumin with a conjugate comprising a fatty acid (FA) covalently linked, optionally through a linker, to an antibody or antigen-binding fragment thereof, wherein the antibody or antigen-binding fragment thereof in the conjugate is capable of specific binding to a target antigen, the FA in the conjugate is capable of binding to albumin, and the binding of albumin to the FA results in a partial or a complete blocking of the binding between the target antigen and the antibody or antigen-binding fragment thereof. In certain embodiments, the contacting step comprises administering a pharmaceutical composition comprising the conjugate to a subject in need of a treatment of a tumor, wherein the tumor comprises the target antigen. In certain embodiments, albumin has a higher turnover rate in the tumor microenvironment compared with the circulating blood, and/or is present in the tumor microenvironment at a level lower than the albumin level in the circulating blood of the subject.

### EMBODIMENTS

The invention provides also the following non-limiting embodiments.

Embodiment 1 is an isolated monoclonal antibody or antigen-binding fragment thereof, wherein the antibody or antigen-binding fragment thereof comprises:
a. a variable heavy chain region (VH);
b. a variable light chain region (VL);
   wherein the antibody or antigen-binding fragment thereof binds to a target antigen, preferably a human target antigen;
   wherein an amino acid residue in the VH, VL, or within a twenty (20)-amino acid distance of the VH or VL on one or both arms is substituted with an amino acid residue that is conjugated to a fatty acid (FA);
   and wherein upon conjugation with the FA at the substituted amino acid residue, the antibody or antigen-binding fragment thereof still binds to the target antigen.

Embodiment 2 is the isolated monoclonal antibody or antigen-binding fragment thereof of embodiment 1, wherein the substituted amino acid residue is within a five (5)-amino acid distance of the VH or VL on one or both arms.

Embodiment 3 is the isolated monoclonal antibody or antigen-binding fragment thereof of embodiment 1 or 2, wherein the substituted amino acid residue is a cysteine residue, a lysine residue, or a modified amino acid that is suitable for chemical conjugation.

Embodiment 4 is the isolated monoclonal antibody or antigen-binding fragment thereof of embodiment 3, wherein the substituted amino acid residue occurs at an amino acid residue corresponding to:
(1) residue 25, 27, 62, 64, 73, 76, 101, 112, or 113 of SEQ ID NO:1 in the VH (Kabat numbering);
(2) residue 26, 27, 52, 53, 56, or 67 of SEQ ID NO:2 in the VL (Kabat numbering);
(3) residue 119 or 120 of SEQ ID NO:9, 10, 11, or 12 in the CH1 (EU numbering); or
(4) residue 121 or 124 of SEQ ID NO:13 or 14 in the CL (EU numbering).

Embodiment 5 is the isolated monoclonal antibody or antigen-binding fragment thereof of embodiment 4, wherein the substituted amino acid residue occurs at an amino acid residue corresponding to:
(1) a K64C substitution of SEQ ID NO:1 in the VH;
(2) a S26C substitution of SEQ ID NO:2 in the VL, or
(3) a T120C substitution of SEQ ID NO:9, 10, 11, or 12 in the CH1 region.

Embodiment 6 is the isolated monoclonal antibody or antigen-binding fragment thereof of any one of embodiments 1 to 5, wherein the antibody or antigen-binding fragment thereof is an anti-immune cell modulator (ICM) antibody or antigen-binding fragment thereof and capable of specific binding to the ICM, preferably a human ICM.

Embodiment 7 is the isolated monoclonal antibody or antigen-binding fragment thereof of embodiment 6, wherein the ICM is selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules.

Embodiment 8 is the isolated monoclonal antibody or antigen-binding fragment thereof of embodiment 7, wherein the ICM is CD3 and the antibody or antigen-binding fragment thereof comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:3, 4, 5, 6, 7, and 8, respectively; or SEQ ID NOs:33, 34, 35, 36, 37, and 38, respectively.

Embodiment 9 is the isolated monoclonal antibody or antigen-binding fragment thereof of embodiment 7 or 8, wherein the ICM is CD3, and wherein the substituted amino acid residue occurs at an amino acid residue selected from:
(1) residue 25, 27, 62, 64, 73, 76, 101, 112, or 113 of SEQ ID NO:1 or 27 in the VH (Kabat numbering);
(2) residue 26, 27, 52, 53, 56, or 67 of SEQ ID NO:2 or 28 in the VL (Kabat numbering);
(3) residue 119 or 120 of SEQ ID NO:9, 10, 11, or 12 in the CH1 (EU numbering); or
(4) residue 121 or 124 of SEQ ID NO:13 or 14 in the CL (EU numbering).

Embodiment 10 is the isolated monoclonal antibody or antigen-binding fragment thereof of any one of embodiments 5 to 9, comprising:
(1) a VH region having a polypeptide sequence of SEQ ID NO:1 with an amino acid substitution of K64C and a VL region having a polypeptide sequence of SEQ ID
(2) a VH region having a polypeptide sequence of SEQ ID NO:27 with an amino acid substitution of K64C and a VL region having a polypeptide sequence of SEQ ID NO:28;
(3) a VH region having a polypeptide sequence of SEQ ID NO:1 and a VL region having a polypeptide sequence of SEQ ID NO:2 with an amino acid substitution of S26C;
(4) a VH region having a polypeptide sequence of SEQ ID NO:27 and a VL region having a polypeptide sequence of SEQ ID NO:28 with an amino acid substitution of S26C;
(5) a CH1 region having a polypeptide sequence selected from SEQ ID NO:9, 10, 11, or 12 with an amino acid substitution of T120C and a CL region having a polypeptide sequence from SEQ ID NO:13 or 14;
(6) a VH region having a polypeptide sequence of SEQ ID NO:1, a VL region having a polypeptide sequence of SEQ ID NO:2, a CH1 region having a polypeptide sequence selected from SEQ ID NO: 9, 10, 11 or 12 with an amino acid substitution of T120C, and a CL region having a polypeptide sequence selected from SEQ ID NO:13 or 14; or
(7) a VH region having a polypeptide sequence of SEQ ID NO:27, a VL region having a polypeptide sequence of SEQ ID NO:28, a CH1 region having a polypeptide sequence selected from SEQ ID NO: 9, 10, 11 or 12 with an amino acid substitution of T120C, and a CL region having a polypeptide sequence selected from SEQ ID NO:13 or 14.

Embodiment 11 is an isolated multi-specific antibody or antigen-binding fragment thereof, wherein the multi-specific antibody or antigen-binding fragment thereof comprises the monoclonal antibody or antigen-binding fragment thereof of any one of embodiments 1 to 10, and wherein the multi-specific antibody or antigen-binding fragment thereof comprises one or more antigen-binding arm(s) comprising a substituted amino acid residue that is conjugated to a FA.

Embodiment 12 is the multi-specific antibody or antigen-binding fragment thereof of embodiment 11, wherein the multi-specific antibody or antigen-binding fragment thereof is a bispecific antibody or antigen-binding fragment comprising a first antigen-binding arm (Ab1) and a second antigen-binding arm (Ab2), wherein Ab1 and/or Ab2 comprises a substituted amino acid that is conjugated to a FA.

Embodiment 13 is the isolated bispecific antibody or antigen-binding fragment thereof of embodiment 12, wherein Ab1 binds an immune cell modulator (ICM), preferably a human ICM.

Embodiment 14 is the isolated bispecific antibody or antigen-binding fragment thereof of embodiment 13, wherein the ICM is selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules.

Embodiment 15 is the isolated bispecific antibody or antigen-binding fragment thereof of any one of embodiments 12 to 14, wherein Ab2 binds a tumor-associated antigen (TAA), preferably a human tumor-associated antigen (human TAA).

Embodiment 16 is the isolated bispecific antibody or antigen-binding fragment thereof of embodiment 15, wherein the TAA is DLL3.

Embodiment 17 is the isolated bispecific antibody or antigen-binding fragment thereof of any one of embodiments 12 to 16, wherein the first antigen-binding arm (Ab1) comprises H1 and L1 and a second antigen-binding arm (Ab2) comprises H2 and L2, wherein:
(a) H1 and H2 each comprises a CH1 region of human IgG1, IgG2, IgG3, or IgG4; and
(b) L1 and L2 each comprises a CL region of a human kappa light chain or a human lambda light chain;
   wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and DN170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) K/R133D/E in CH1 of H1 and K207K/R in CL of L1, respectively, and K/R133K/R in CH1 of H2 and K207D/E in CL of L2, respectively;
(6) K/R133D/E in CH1 of H1 and I/L117K/R in CL of L1, respectively, and K/R133K/R in CH1 of H2 and I/L117D/E in CL of L2, respectively;
(7) K/R133D/E in CH1 of H1 and F/V209K/R in CL of L1, respectively, and K/R133K/R in CH1 of H2 and F/V209D/E in CL of L2, respectively;
(8) G166D/E in CH1 of H2 and S114K/R in CL of L2, respectively, and G166K/R in CH1 of H1 and S114D/E in CL of L1, respectively;
(9) T187D/E in CH1 of H2 and DN170K/R in CL of L2, respectively, and T187K/R in CH1 of H1 and D/N170D/E in CL of L1, respectively;
(10) S131D/E in CH1 of H2 and P119K/R in CL of L2, respectively, and S131K/R in CH1 of H1 and P119D/E in CL of L1, respectively;
(11) A129D/E in CH1 of H2 and S121K/R in CL of L2, respectively, and A129K/R in CH1 of H1 and S121D/E in CL of L1, respectively;
(12) K/R133D/E in CH1 of H2 and K207K/R in CL of L2, respectively, and K/R133K/R in CH1 of H1 and K207D/E in CL of L1, respectively;
(13) K/R133D/E in CH1 of H2 and I/L117K/R in CL of L2, respectively, and K/R133K/R in CH1 of H1 and I/L117D/E in CL of L1, respectively; or
(14) K/R133D/E in CH1 of H2 and F/V209K/R in CL of L2, respectively, and K/R133K/R in CH1 of H1 and F/V209D/E in CL of L1, respectively.

Embodiment 18 is the isolated bispecific antibody or antigen-binding fragment thereof of any one of embodiments 12 to 17, wherein the bispecific antibody or antigen-binding fragment thereof comprises:
a. a first antigen-binding arm (Ab1) comprising a VH region having a polypeptide sequence of SEQ ID NO:15, a VL region having a polypeptide sequence of SEQ ID NO:17, a CH1 region having a polypeptide sequence of SEQ ID NO:16, and a CL region having a polypeptide sequence of SEQ ID NO:18;
b. a first antigen-binding arm (Ab1) comprising a VH region having a polypeptide sequence of SEQ ID NO:19, a VL region having a polypeptide sequence of SEQ ID NO:21, a CH1 region having a polypeptide sequence of SEQ ID NO:20, and a CL region having a polypeptide sequence of SEQ ID NO:22;
c. a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18; or
d. a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22.

Embodiment 19 is the isolated bispecific antibody or antigen-binding fragment thereof of embodiment 18, wherein the second antigen-binding arm (Ab2) comprises a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26.

Embodiment 20 is the isolated antibody or antigen-binding fragment thereof of any one of embodiments 1 to 19, wherein the FA is selected from a FA with 6 carbons, 8 carbons, 10 carbons, 12 carbons, 14 carbons, 16 carbons, or 18 carbons, or any number of carbons in between.

Embodiment 21 is the isolated antibody or antigen-binding fragment thereof of embodiment 20, wherein the FA is selected from a FA with 14 carbons or 18 carbons or any number of carbons in between.

Embodiment 22 is the isolated antibody or antigen-binding fragment thereof of any one of embodiments 1 to 21, wherein the FA comprises a linker for conjugation to the substituted amino acid residue.

Embodiment 23 is the isolated antibody or antigen-binding fragment thereof of embodiment 22, wherein the linker is selected from a peptide linker or a polyethylene glycol linker.

Embodiment 24 is the isolated antibody or antigen-binding fragment thereof of embodiment 23, wherein the peptide linker is less than 50 amino acids.

Embodiment 25 is the isolated antibody or antigen-binding fragment thereof of any one of embodiments 1 to 24, wherein the FA conjugated to the antibody or antigen-binding fragment thereof is capable of binding albumin, wherein the binding of albumin to the FA results in a partial or a complete blocking of the binding between the target antigen and the antibody or antigen-binding fragment thereof.

Embodiment 26 is the isolated antibody or antigen-binding fragment thereof of any one of embodiments 1 to 25, wherein the isolated antibody or antigen-binding fragment thereof has reduced ability to activate T cells upon binding to albumin as compared to the isolated antibody or antigen-binding fragment thereof not binding to albumin.

Embodiment 27 is an isolated nucleic acid encoding the isolated antibody or antigen-binding fragment thereof of any one of embodiments 1 to 26.

Embodiment 28 is a vector comprising the isolated nucleic acid of embodiment 27.

Embodiment 29 is an isolated host cell comprising the vector of embodiment 27.

Embodiment 30 is a pharmaceutical composition comprising the isolated antibody or antigen-binding fragment thereof of any one of embodiments 1 to 26, and a pharmaceutically acceptable carrier.

Embodiment 31 is a method of treating a cancer in a subject in need thereof, the method comprising administering to the subject the pharmaceutical composition of embodiment 30.

Embodiment 32 is the method of embodiment 31, wherein the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 33 is a method of producing the isolated antibody or antigen-binding fragment thereof of any one of embodiments 1 to 26, the method comprising culturing a cell comprising a nucleic acid encoding the antibody or antigen-binding fragment thereof under conditions to produce the antibody or antigen-binding fragment thereof, and recovering the antibody or antigen-binding fragment thereof from the cell or culture.

Embodiment 34 is the method of embodiment 33, further comprising conjugating the FA to the antibody or antigen-binding fragment thereof at the substituted amino acid residue.

Embodiment 35 is a method of producing a pharmaceutical composition comprising the isolated antibody or antigen-binding fragment thereof, the method comprising combining the antibody or antigen-binding fragment thereof of any one of embodiments 1 to 26 with a pharmaceutically acceptable carrier to obtain the pharmaceutical composition.

Embodiment 36 is a method, comprising contacting albumin with the isolated antibody or antigen-binding fragment thereof of any one of embodiments 1 to 26, wherein the antibody or antigen-binding fragment thereof is capable of specific binding to a target antigen, the FA is capable of binding to albumin, and the binding of albumin to the FA results in a partial or a complete blocking of the binding between the target antigen and the antibody or antigen-binding fragment thereof.

Embodiment 37 is the method of embodiment 36, wherein the contacting step comprises administering a pharmaceutical composition comprising the isolated antibody or antigen-binding fragment thereof to a subject in need of a treatment of a tumor, wherein the tumor comprises the target antigen.

Embodiment 38 is the method of embodiment 36 or 37, wherein albumin has a higher turnover rate in the tumor microenvironment compared with the circulating blood, and/or is present in the tumor microenvironment at a level lower than the albumin level in circulating blood of the subject, preferably, the lower level of albumin in the tumor microenvironment is due to a high albumin catabolism in the tumor microenvironment and/or a high level of proteases in the tumor microenvironment.

### EXAMPLES

### Example 1: Construction and Characterization of Monoclonal Antibodies for Conjugation with Fatty Acid Molecules

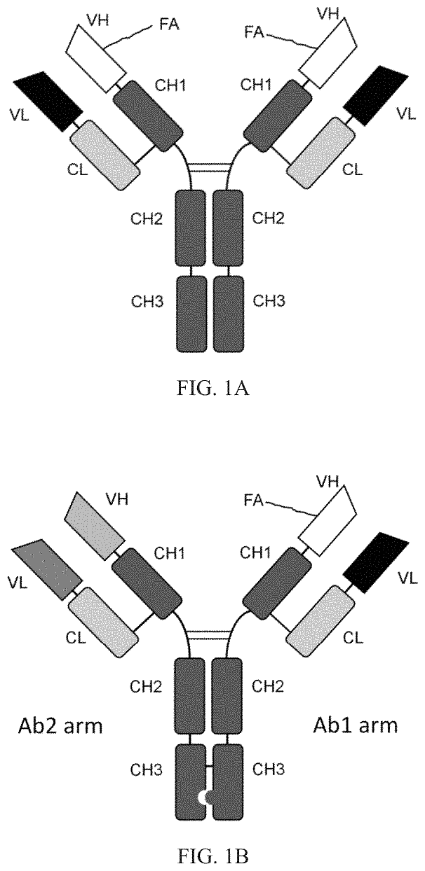

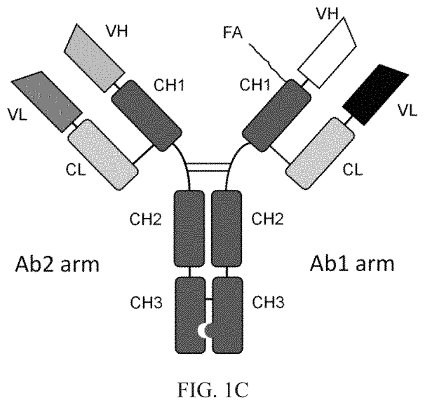

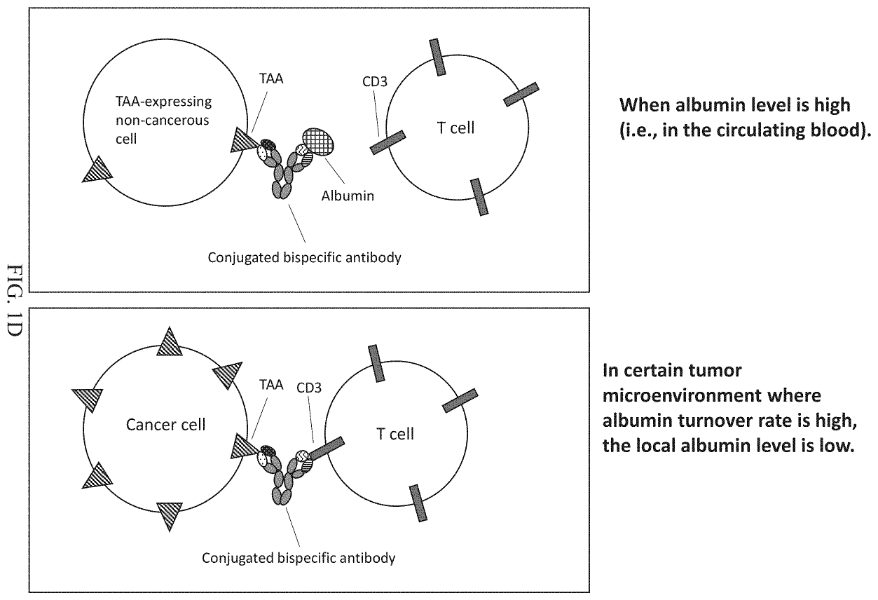

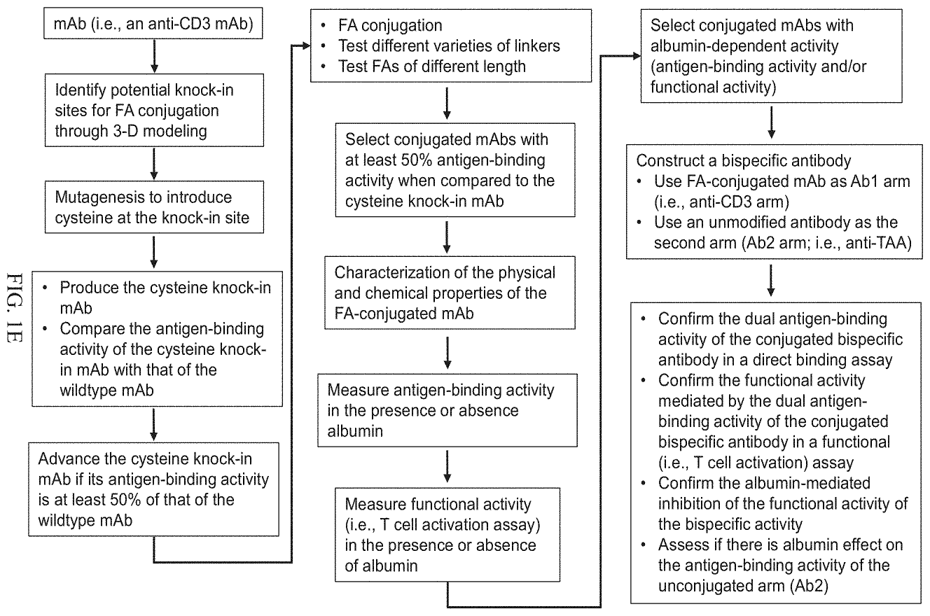

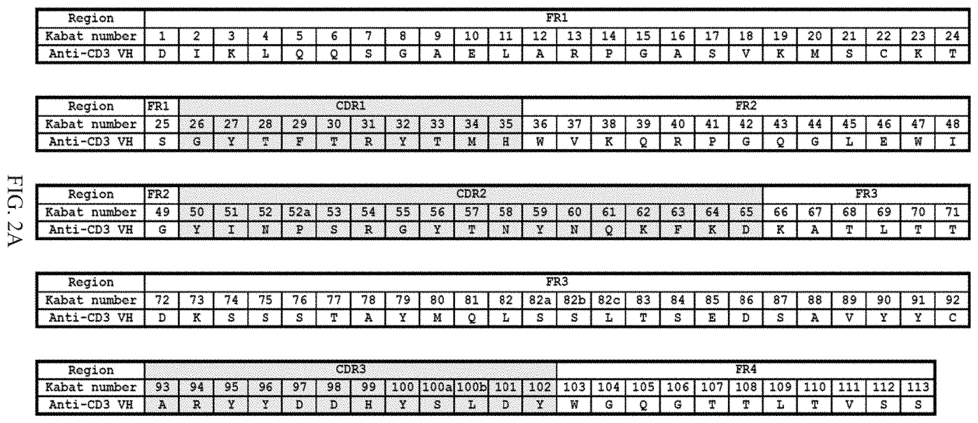

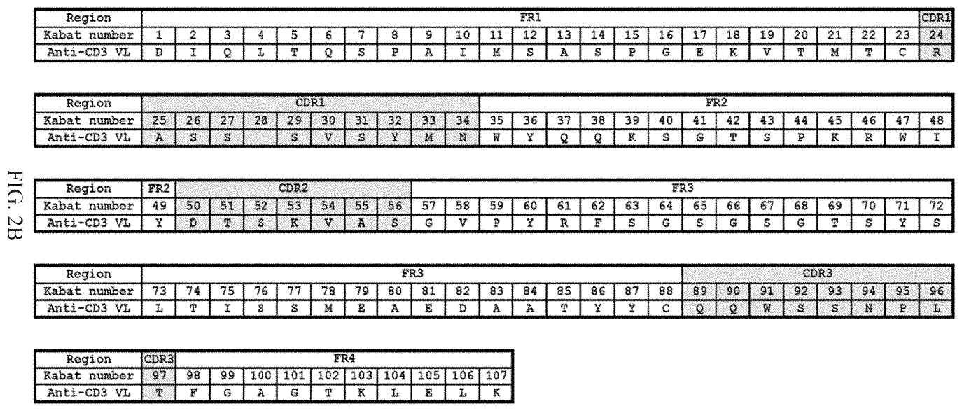

FIG. 1A illustrates a schematic of a monoclonal antibody (mAb) where a residue in the VH region is identified and substituted with a cysteine (the knocked in cysteine). A fatty acid (FA) molecule comprising a linker and a reactive group is conjugated to the knocked in cysteine so that each mAb contains two FA molecules (FIG. 1A). A monoclonal antibody can also comprise a substituted amino acid residue in the VL or within a twenty (20)-amino acid distance, preferably a five (5)-amino acid distance, of the VH or VL in the CH1 or CL region. The knocked in cysteine residue can also be another reactive amino acid residue that is suitable for FA conjugation.

The conjugated FA molecules can bind to albumin circulating in the blood and/or interstitial fluids in tissues. The bound albumin molecules are expected to partially or completely block the interaction of the antigen-binding domain (comprising the VH and VL) of the conjugated mAb with the antigen due to the steric hinderance effect of the bound albumin. Hence, the antigen-binding activity of the conjugated mAb is capable of being regulated by the surrounding albumin level. Depending on the location of the knocked in amino acid residue, the length of the FA, the presence of a linker, and the length of the linker, different degrees of modulation of the mAb binding to the target antigen can be achieved.

The mAb in FIG. 1A can, for example, be an anti-CD3 antibody. After conjugation, the activity of the anti-CD3 mAb can be modulated in vivo by albumin, such that the anti-CD3 mAb is inactive or less active in the circulating blood. In the tumor microenvironment, due to the higher turnover rate of albumin compared with the circulating blood, the concentration of the naked conjugated mAb (i.e., the non-albumin bound conjugated anti-CD3 mAb) increases and the activation of T cells results in a cancer killing effect mediated by the anti-CD3 mAb. The mAb can be directed to other cancer targets especially ICMs (e.g., 4-1BB, GITR, OX40, CD28 or PD-1) where the therapeutic approach requires less or no activity of the mAb in the circulating blood and more activity in the tumor microenvironment. Further, the conjugation and modulation strategy can be used with an antigen-binding fragment that is not a mAb. In this case, the FA conjugation site, the length of the FA, the presence of the linker, and the length of the linker are selected so that the FA-bound albumin protrudes into the interface between the antigen-binding domain and the target antigen.

The conjugated mAb or an antigen-binding fragment thereof can be used to construct a bispecific antibody (bsAb) or antigen-binding fragment thereof with another antibody or antigen-binding fragment thereof. For illustration purposes, conjugated bispecific antibodies are shown in FIGS. 1B-1C. The Ab1 arm is from an anti-CD3 antibody, and the Ab2 arm is from a mAb against a tumor-associated antigen (TAA). Conjugation of a FA molecule to a region (e.g., a VH, VL, or within a twenty (20)-amino acid distance, preferably a five (5)-amino acid distance, of the VH or VL region) of the anti-CD3 arm can modulate the anti-CD3 activity, and, hence, modulate T cell activation by the bispecific antibody, while the binding of the anti-TAA arm to the TAA is unaffected by surrounding albumin concentrations. The FA-conjugated bispecific antibody is expected to be less or not active in the circulating blood and/or certain tissue fluids where there are high albumin levels (e.g., 35 to 50 mg/mL). The FA-conjugated bispecific antibody is expected to be active in stimulating T cells in certain tumor microenvironments due to the high turnover rate of albumin, which results in lower local albumin levels and higher concentrations of the naked conjugated bsAb (i.e., the non-albumin bound conjugated anti-CD3 bsAb) and increased cancer cell killing by the activation of T cells. The anti-CD3 arm of the bsAb can be directed to other cancer targets especially ICMs (e.g., 4-1BB, GITR, OX40, CD28 or PD-1) where the therapeutic approach requires less or no activity of the bsAb in the circulating blood and more activity in the tumor microenvironment. This approach can increase the safety margin of an anti-CD3 based bispecific T cell engager by minimizing on-target, off-tumor toxicities. Such a therapeutic can reduce the risk of cytokine storm syndrome (CRS) usually observed with anti-CD3 T cell engagers. The conjugation and modulation strategy can be used with a bispecific antigen-binding fragment that is not a bispecific antibody. In this case, the FA conjugation site, the length of the FA, the presence of a linker, and the length of the linker are selected so that the FA-bound albumin protrudes into the interface between the antigen-binding domain and the target antigen.

FIG. 1D provides a schematic demonstrating the mechanism of action for a FA-conjugated bispecific antibody T cell engager killing a cancer cell. The anti-TAA arm binds to the TAA on the cancer cell surface regardless of the surrounding albumin level. The FA-conjugated T cell engaging arm (e.g., the anti-CD3 arm) does not bind to the target antigen (e.g., CD3) when the surrounding albumin level is high (e.g., 35 to 50 mg/mL, e.g., in the circulating blood); however, when the surrounding albumin level is low, the FA-conjugated T cell engaging arm (e.g., the anti-CD3 arm) binds to the target antigen (e.g., CD3), and activates the T cell, which results in the death of the cancer cell. The FA-conjugated arm can be a T cell engaging arm against other T cell ICMs, such as 4-1BB, GITR, OX40, CD28, PD-1, or any other targets that are expressed on T cells and can mediate T cell activation upon binding by a specific antibody. Further, the FA-conjugated arm can be against ICMs on other immune cells. The approach of leveraging the lower albumin level on the target site compared with that in the circulating blood can also be applied to therapies that target tissues where the local albumin level is low; these tissues include adipose tissue and skeletal muscle (Ellmerer et al., Am J Physiol Endocrinol Metab. 2000. 278: E352-E356). FIG. 1E shows the specific steps for identifying a FA-conjugated mAb or bsAb.

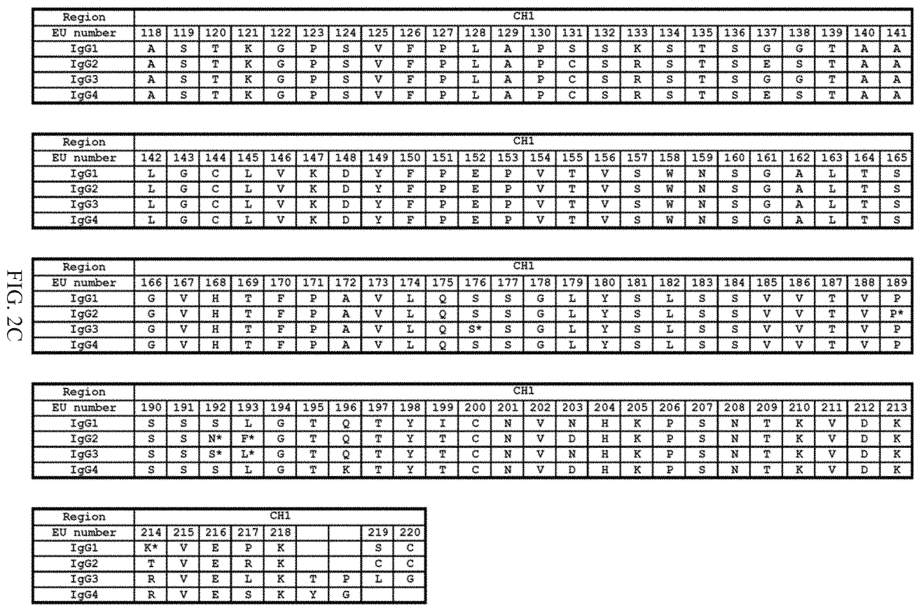

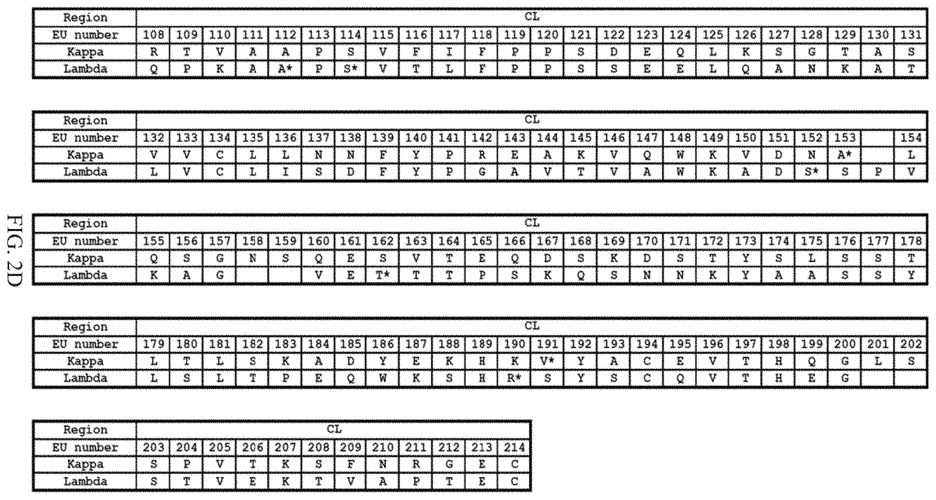

A modified anti-CD3 antibody was used to construct conjugated mAbs. The sequences and numberings of the VH and VL regions of the anti-CD3 mAb are shown in FIGS. 2A-2B and Table 1 (SEQ ID NOs:1 and 2, respectively). The sequences of the CDR regions (HCDR1, HCDR2, HCDR3, LCDR1, LCDR2, and LCDR3) are listed in Table 2 (SEQ ID NOs:3, 4, 5, 6, 7 and 8, respectively, and SEQ ID Nos:33, 34, 35, 36, 37, and 38, respectively). The sequences and numberings of the CH1 regions of IgG1, IgG2, IgG3, and IgG4 heavy chains (HCs) are shown in FIG. 2C and Table 1 (SEQ ID NOs:9, 10, 11, and 12, respectively). The sequences and numberings of the CL regions of kappa and lambda light chains (LCs) are shown in FIG. 2D and Table 1 (SEQ ID NOs:13 and 14, respectively).

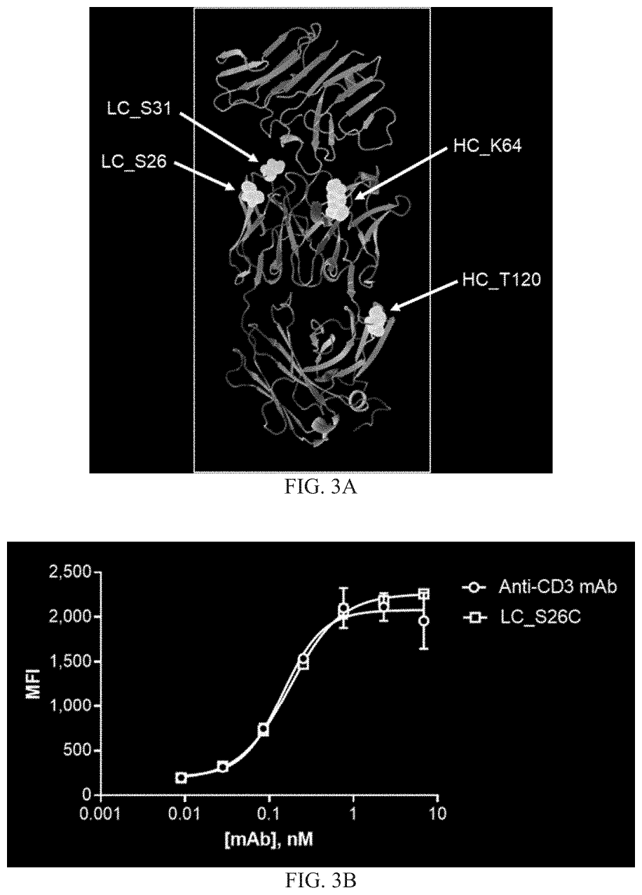

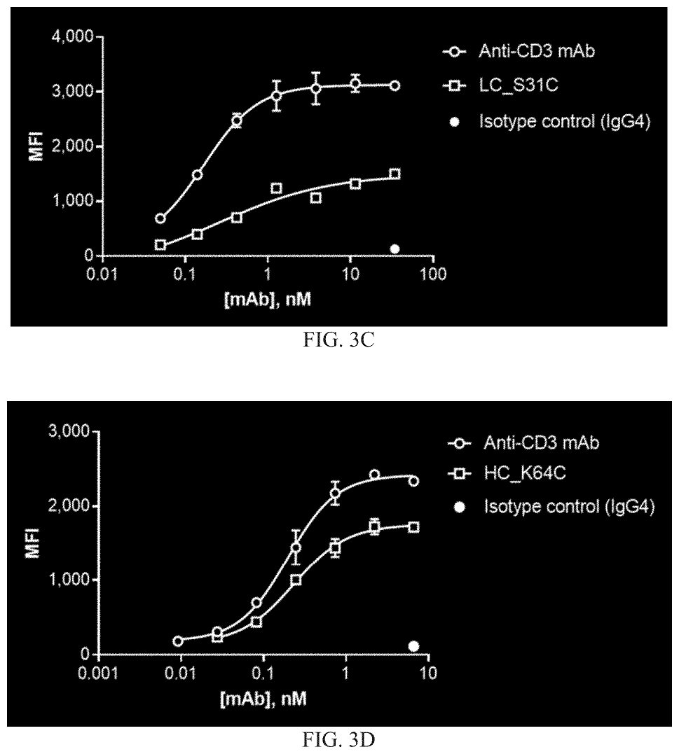

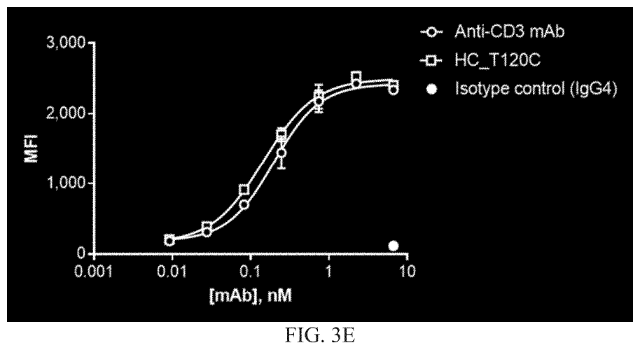

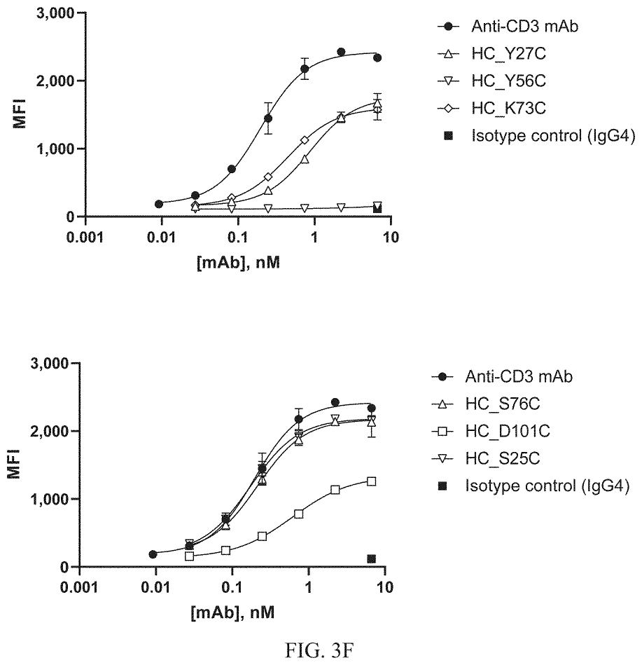

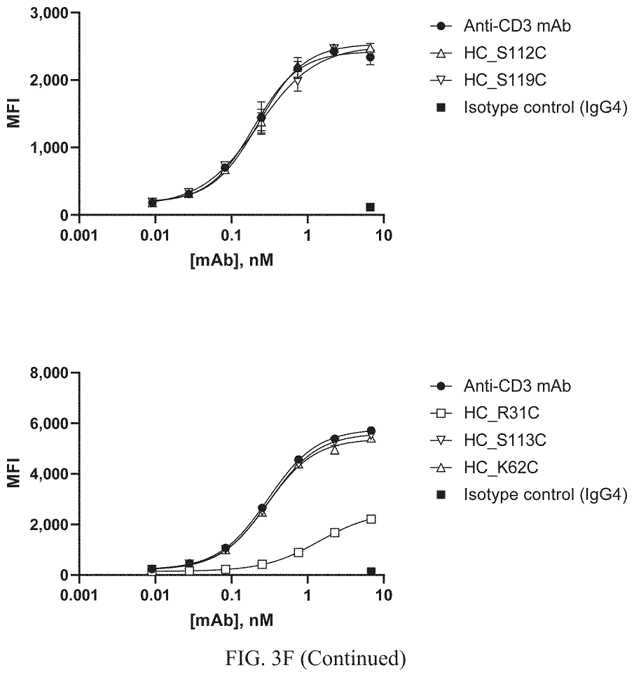

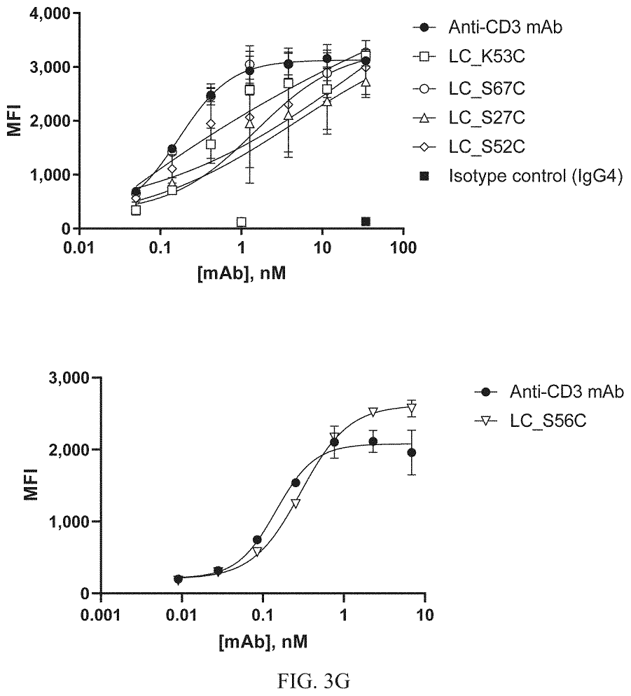

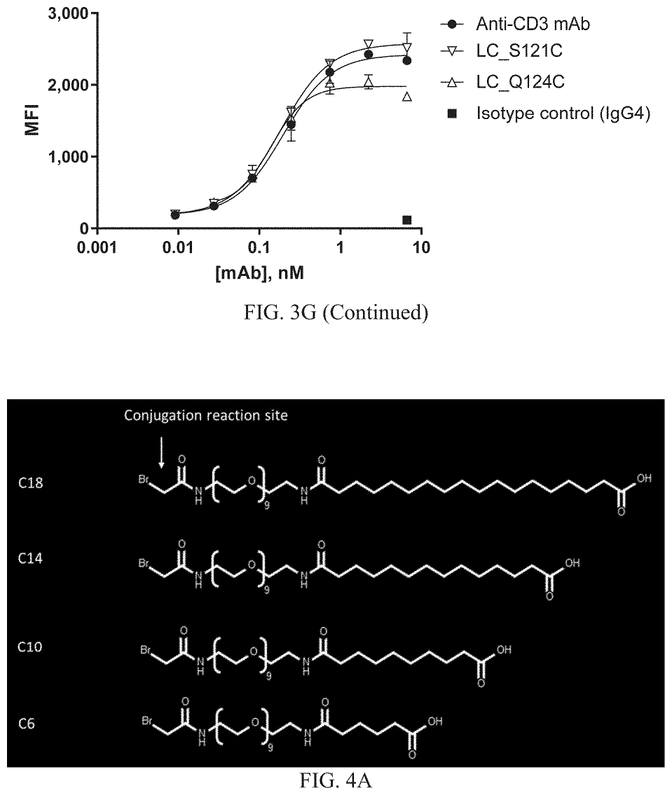

Residues on the surface of the Fab region of the anti-CD3 antibody were identified using structural modeling. The sequence of the anti-CD3 mAb was modeled to 1SY6 and 3EO9 by Schrodinger Bioluminate® (Schrodinger; New York, N.Y.). Side chain solvent accessibility was calculated and residues in or near the variable regions of both heavy and light chains with side chain accessibility ranging from 30%-70% were selected as possible cysteine knock-in candidates. Residues identified for knock-in are listed in Table 3. Four residues selected for cysteine knock-in experiment are shown in a 3-D structure of the anti-CD3 mAb as examples (FIG. 3A): S26 and S31 in the VL region, K64 in the VH region, and T120 in the CH1 region of the HC (3-amino acid residues away from the C-terminus of the VH region). The mAbs with cysteines knocked into these sites were named LC_S26C, LC_S31C, HC_K64C, and HC_T120C, respectively. LC_S26C represents the anti-CD3 mAb with the serine residue in the S26 position on the light chain substituted with cysteine. The other mAbs follow the same naming rule.

TABLE 1
Sequences of the anti-CD3 VH, anti-CD3 VL, #2 anti-CD3 VH, #2 anti-
CD3 VL, IgG1 CH1, IgG2 CH1, IgG3 CH1, IgG4 CH1, Kappa CL and Lambda CL
regions

| | | SEQ |
|---|---|---|
| | | ID |
| Region name | Sequence | NO: |
| Anti-CD3 VH | DIKLQQSGAELARPGASVKMSCKTSGYTFTRYTMHWVKQRPGQGLEWI | 1 |
| | GYINPSRGYTNYNQKFKDKATLTTDKSSSTAYMQLSSLTSEDSAVYYCA | |
| | RYYDDHYSLDYWGQGTTLTVSS | |
| Anti-CD3 VL | DIQLTQSPAIMSASPGEKVTMTCRASSSVSYMNWYQQKSGTSPKRWIYD | 2 |
| | TSKVASGVPYRFSGSGSGTSYSLTISSMEAEDAATYYCQQWSSNPLTFGA | |
| | GTKLELK | |
| #2 anti-CD3 VH | QVQLQQSGAELARPGASVKMSCKASGYTFTRYTMHWVKQRPGQGLEW | 27 |
| | IGYINPSRGYTNYNQKFKDKATLTTDKSSSTAYMQLSSLTSEDSAVYYC | |
| | ARYYDDHYSLDYWGQGTTLTVSS | |
| #2 anti-CD3 VL | QIVLTQSPAIMSASPGEKVTMTCSASSSVSYMNWYQQKSGTSPKRWIYD | 28 |
| | TSKLASGVPAHFRGSGSGTSYSLTISGMEAEDAATYYCQQWSSNPFTFGS | |
| | GTKLEIN | |
| IgGl CH1 | ASTKGPSVFPLAPSSKSTSGGTAALGCLVKDYFPEPVTVSWNSGALTSGV | 9 |
| | HTFPAVLQSSGLYSLSSVVTVPSSSLGTQTYICNVNHKPSNTKVDKKVEP | |
| | KSC | |
| IgG2 CH1 | ASTKGPSVFPLAPCSRSTSESTAALGCLVKDYFPEPVTVSWNSGALTSGV | 10 |
| | HTFPAVLQSSGLYSLSSVVTVPSSNFGTQTYTCNVDHKPSNTKVDKTVE | |
| | RKCC | |
| IgG3 CH1 | ASTKGPSVFPLAPCSRSTSGGTAALGCLVKDYFPEPVTVSWNSGALTSG | 11 |
| | VHTFPAVLQSSGLYSLSSVVTVPSSSLGTQTYTCNVNHKPSNTKVDKRV | |
| | ELKTPLG | |
| IgG4 CH1 | ASTKGPSVFPLAPCSRSTSESTAALGCLVKDYFPEPVTVSWNSGALTSGV | 12 |
| | HTFPAVLQSSGLYSLSSVVTVPSSSLGTKTYTCNVDHKPSNTKVDKRVES | |
| | KYG | |
| Kappa CL | RTVAAPSVFIFPPSDEQLKSGTASVVCLLNNFYPREAKVQWKVDNALQS | 13 |
| | GNSQESVTEQDSKDSTYSLSSTLTLSKADYEKHKVYACEVTHQGLSSPV | |
| | TKSFNRGEC | |
| Lambda CL | QPKAAPSVTLFPPSSEELQANKATLVCLISDFYPGAVTVAWKADSSPVK | 14 |
| | AGVETTTPSKQSNNKYAASSYLSLTPEQWKSHRSYSCQVTHEGSTVEKT | |
| | VAPTEC | |

TABLE 2
CDR regions for anti-CD3 mAb and #2 anti-CD3 mAb

| | | SEQ | | SEQ | | SEQ |
|---|---|---|---|---|---|---|
| | | ID | | ID | | ID |
| Chain | CDR1 | NO: | CDR2 | NO: | CDR3 | NO: |
| HC | GYTFTRYTMH | 3 | YINPSRGYTNYNQKFKD | 4 | ARYYDDHYSLDY | 5 |
| LC | RASSSVSYMN | 6 | DTSKVAS | 7 | QQWSSNPLT | 8 |
| #2 | GYTFTRYTMH | 33 | YINPSRGYTNYNQKFKD | 34 | ARYYDDHYSLDY | 35 |
| HC | | | | | | |
| #2 | SASSSVSYMN | 36 | DTSKLAS | 37 | QQWSSNPFT | 38 |
| LC | | | | | | |

HC: heavy chain; LC: light chain; CDR: complementarity determining region; the CDRs for the anti-CD3 mAh were determined utilizing a combination of IMGT (Lefranc, M.-P. et al., Nucleic Acids Res. 1999; 27:209-212) and Kabat methods (Elvin A. Kabat et al, Sequences of Proteins of Immunological Interest 5th ed. (1991)).

TABLE 3
Candidate amino acid substitutions
on the anti-CD3 mAb for conjugation

| | |
|---|---|
| Cysteine | Region |

| knock-in site | VH | CH1 | VL | CL |
|---|---|---|---|---|
| 1 | | | S26C | |
| 2 | K64C | | | |
| 3 | | T120C | | |
| 4 | | | S67C | |
| 5 | | | | S121C |
| 6 | | | | Q124C |
| 7 | | S119C | | |
| 8 | S112C | | | |
| 9 | S25C | | | |
| 10 | S76C | | | |
| 11 | | | S27C | |
| 12 | | | S52C | |
| 13 | | | S56C | |
| 14 | K62C | | | |
| 15 | S113C | | | |
| 16 | | | K53C | |
| 17 | K73C | | | |
| 18 | Y27C | | | |
| 19 | D101C | | | |
| 20 | Q61 | | | |
| 21 | | | Q3 | |
| 22 | | | T5 | |
| 23 | | | S7 | |
| 24 | | | K18 | |
| 25 | | | T20 | |
| 26 | | | K45 | |
| 27 | | | Y60 | |
| 28 | | | S63 | |
| 29 | | | S65 | |
| 30 | | | S76 | |
| 31 | K3 | | | |
| 32 | Q5 | | | |
| 33 | K19 | | | |
| 34 | T68 | | | |
| 35 | T70 | | | |
| 36 | S74 | | | |
| 37 | S75 | | | |
| 38 | S82a | | | |
| 39 | Q105 | | | |
| 40 | | | A9 | |
| 41 | | | I10 | |
| 42 | | | S14 | |
| 43 | | | P15 | |
| 44 | | | G16 | |
| 45 | | | E17 | |
| 46 | | | T42 | |
| 47 | | | S77 | |
| 48 | | | A80 | |
| 49 | | | E81 | |
| 50 | | | A100 | |
| 51 | | | K107 | |
| 52 | A9 | | | |
| 53 | L11 | | | |
| 54 | R13 | | | |
| 55 | A16 | | | |
| 56 | S17 | | | |
| 57 | P41 | | | |
| 58 | Q43 | | | |
| 59 | S82b | | | |
| 60 | T83 | | | |
| 61 | S84 | | | |
| 62 | E85 | | | |
| 63 | T108 | | | |
| 64 | T110 | | | |
| 65 | D1 | | | |
| 66 | I2 | | | |
| 67 | N58 | | | |
| 68 | T71 | | | |
| 70 | | | V54 | |

Note:
the mAbs shown in bold have been produced and shown to have significant CD3 binding activity (maximum binding is greater than 50% of the maximum antigen-binding activity of the wildtype anti-CD3 mAb) after cysteine knock-in. The residues where the effect of amino acid substitution on the antigen-binding activity has not been tested are shown in regular form. VH and VL, Kabat numbering; CH1 and CL, EU numbering.

The four mAbs LC_S26C, LC_S31C, HC_K64C, and HC_T120C in the human IgG4 HC and human kappa LC framework were expressed in CHO cells and purified using Protein A chromatography and tested for CD3 binding by FACS using Jurkat cells (FIGS. 3B-3E). LC_S31C lost significant activity after cysteine knock in (FIG. 3C). LC_S26C, HC_K64C and HC_T120C had substantial activity in CD3 binding (FIG. 3B and FIGS. 3D-3E) and were selected for further studies. Cysteine knock-in was also carried out at additional residues as shown in Table 3. The resulting mAbs in the IgG4 HC and kappa LC framework were expressed in CHO cells, purified using protein A chromatography and tested for CD3 binding by FACS using Jurkat cells. The residues after cysteine knock-in maintained greater than 50% of the maximum antigen-binding activity of the wildtype anti-CD3 mAb are shown in bold form in Table 3 and the results for CD3 binding by FACS using Jurkat cells are shown in FIGS. 3F-3G. The residues where the effect of amino acid substitution on the antigen-binding activity has not been tested are shown in regular form (Table 3).

### Example 2: Characterization of Monoclonal Antibodies Conjugated with Fatty Acid Molecules

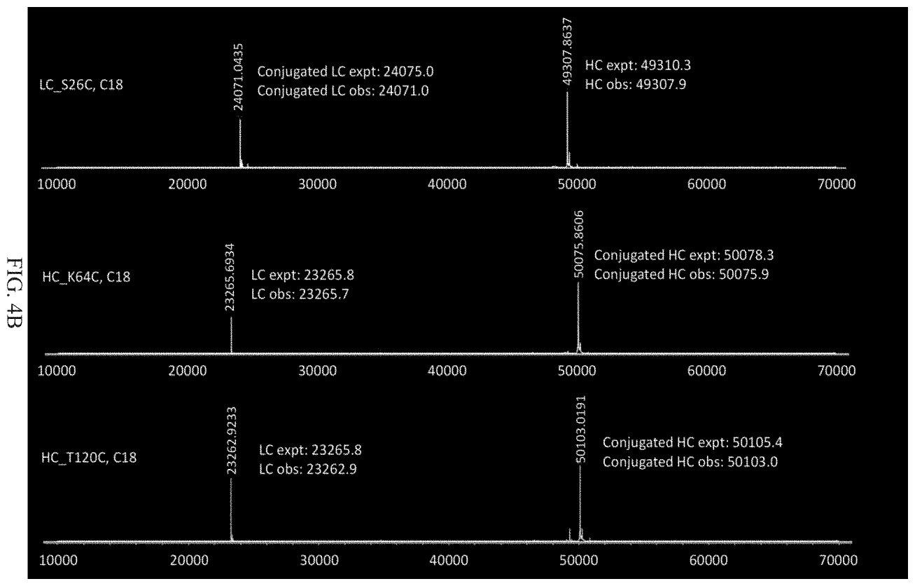

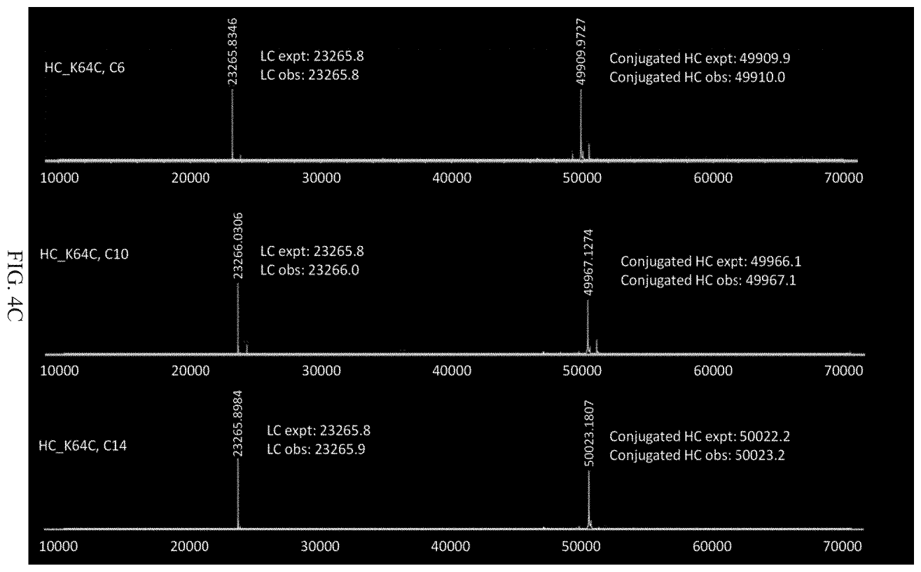

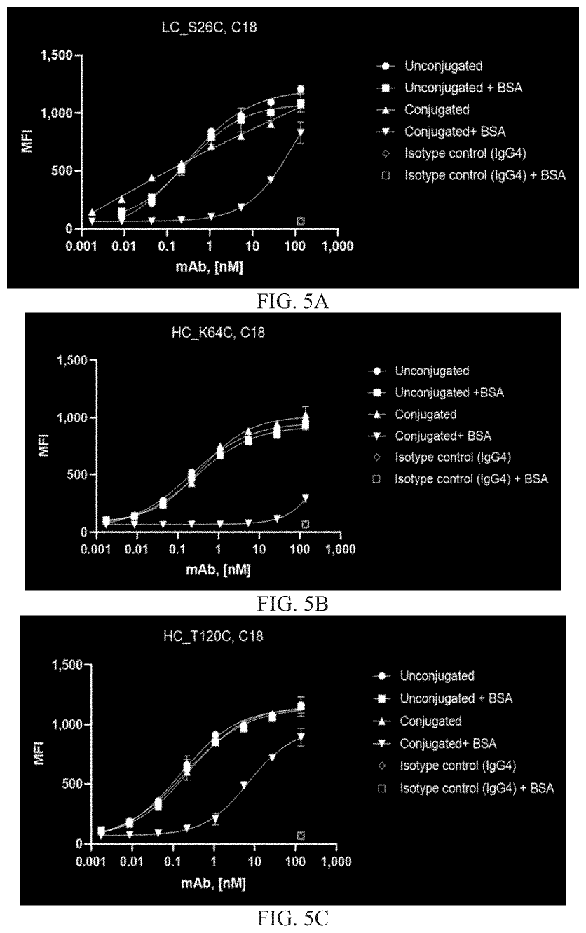

The FA molecules used for conjugation are shown in FIG. 4A, including C18, C14, C10, and C6. All molecules contain a PEG linker and a bromoacetamide reactive group. For the conjugation reaction, the antibody was concentrated to a concentration of 20-30 mg/mL and buffer exchanged into TBS buffer. The antibody was partially reduced with the addition of 10 equivalents of tris (2-carboxyethyl) phosphine (TCEP), and the solution was incubated for 1 hour at 37° C. The antibody was then buffer exchanged into DPBS and re-oxidized with 30 equivalents of dehydroascorbic acid by incubating for 1 hour at room temperature (RT). The antibody was buffer exchanged into conjugation buffer (20 mM Tris pH 8.5+150 mM NaCl+10% glycerol) and diluted to a concentration of 10 mg/mL. The FA molecule was added at 20 equivalents from a 50 mM solution in DMSO and the resulting mixture was incubated at RT for 1 or 2 days. The final product was buffer exchanged into conjugation buffer to remove unreacted FA molecules. The samples were purified by hydrophobic interaction chromatography and analyzed with liquid chromatography/mass spectrometry (LC/MS). The correct conjugate on the HC or LC was confirmed using mass spectrometry (MS) for each conjugated mAb (FIGS. 4B-4C). The conjugation of the FA to the right cysteine knock-in site was confirmed by LC/MS (Table 4).

TABLE 4
Confirmation of the conjugation of a FA
to the right cysteine knock-in sites

| | m/z | m/z | m/z | m/z | m/z | m/z |
|---|---|---|---|---|---|---|
| | (z = 2) | (z = 2) | (z = 3) | (z = 3) | (z = 4) | (z = 4) |
| mAb + FA | (expt) | (obs) | (expt) | (obs) | (expt) | (obs) |
| LC_S26C + | 1244.99 | 1244.15 | 830.33 | 829.76 | | |
| C18 | | | | | | |
| HC_K64C + | 653.34 | 651.86 | | | | |
| C18 | | | | | | |
| HC_T120C + | | | 1219.05 | 1218.93 | 914.53 | 914.45 |
| C18 | | | | | | |
| HC_K64C + | 569.14 | 568.77 | | | | |
| C6 | | | | | | |
| HC_K64C + | 597.24 | 596.81 | | | | |
| C10 | | | | | | |
| HC_K64C + | 625.29 | 624.84 | | | | |
| C14 | | | | | | |

Note:
Conjugated mAbs were subjected to trypsin digestion and analyzed with LC/MS. For a given conjugated mAb, the peak corresponding to the peptide fragment with the FA conjugated to the correct cysteine knock-in site was identified on LC/MS. m/z, mass-to-charge ratio where m is mass and z is the number of charges; expt, expected; obs, observed.

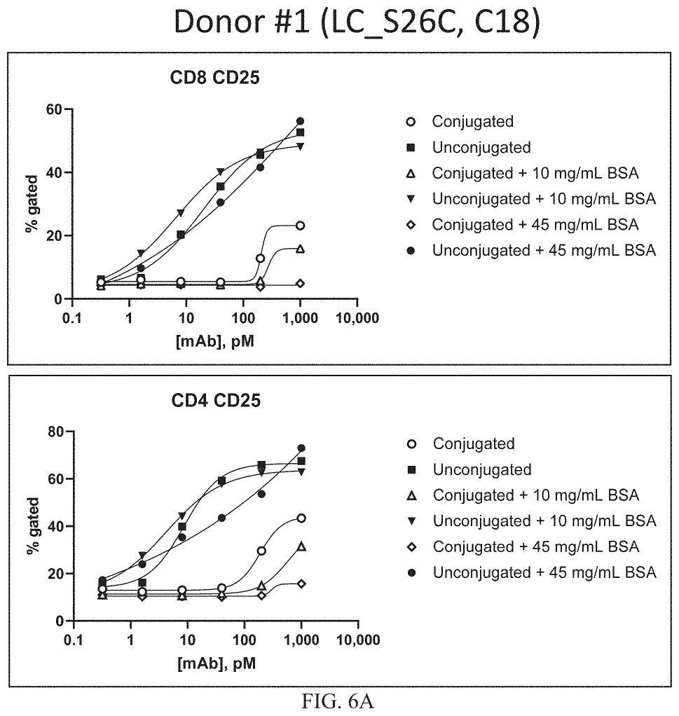

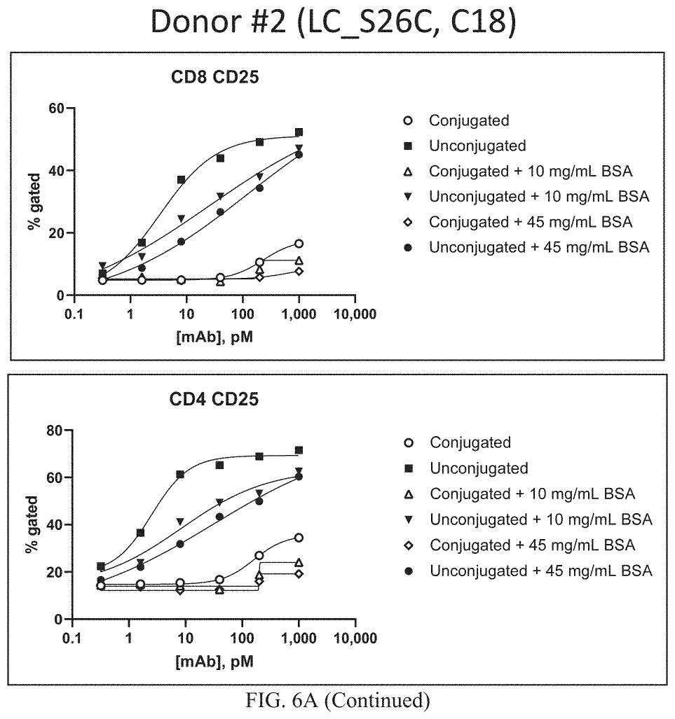

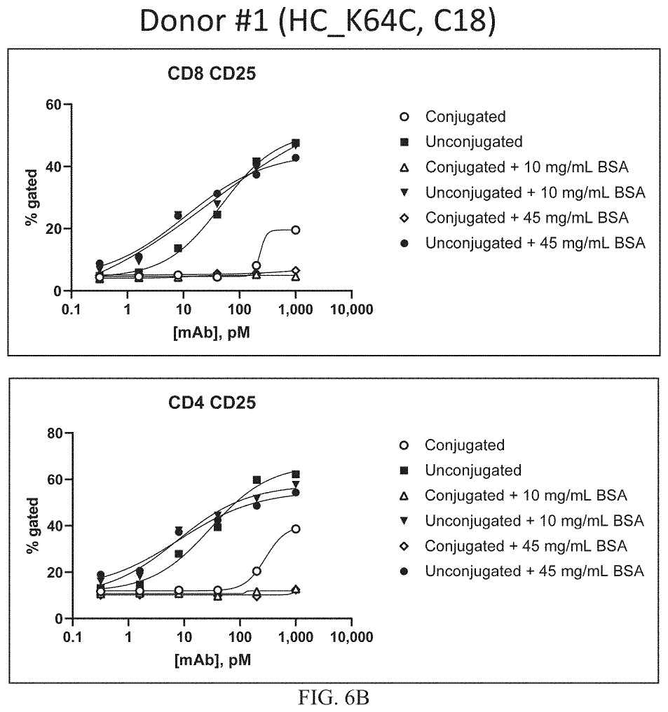

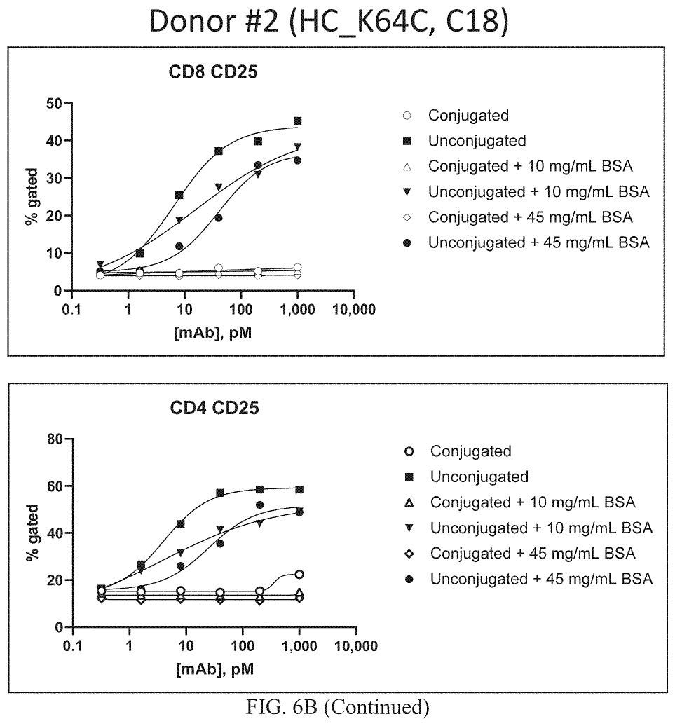

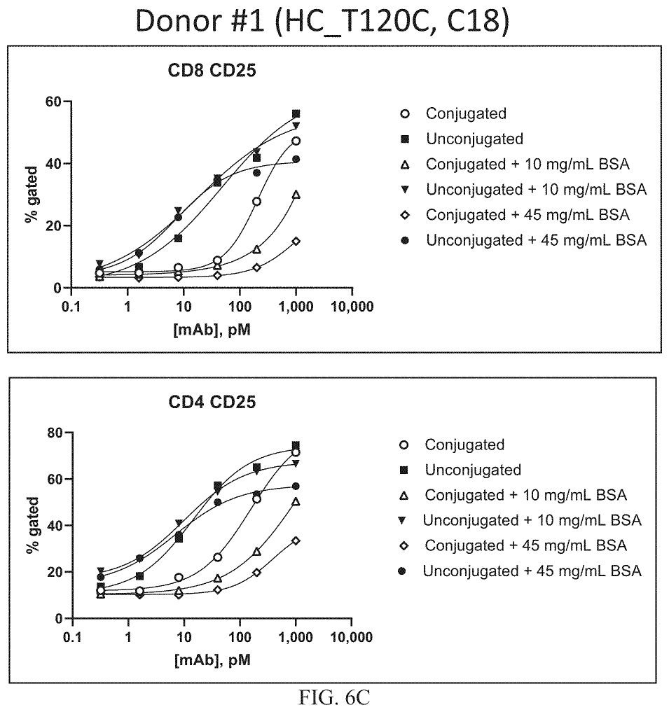

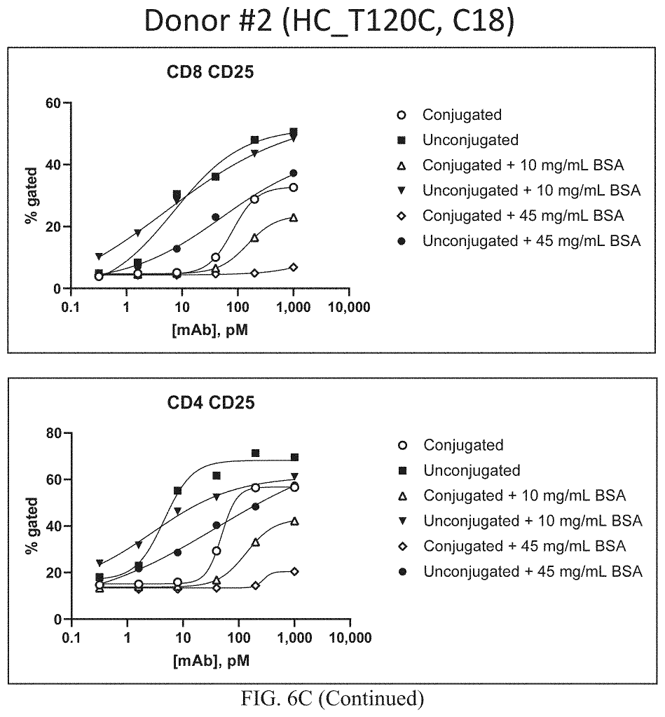

The C18-conjugated mAbs were tested for their ability to bind to Jurkat cells (which are known to express CD3) in the absence or presence of 50 mg/mL bovine serum albumin (BSA) (FIGS. 5A-5C). Jurkat cells were incubated with the indicated antibodies in HBSS buffer containing 0.1% casein with or without BSA during the primary antibody binding step and were subsequently processed in BSA free buffer. Antibody binding was quantified by FACS. The binding of the unconjugated mAbs to Jurkat cells was demonstrated, and the binding was not affected by the presence of BSA (FIGS. 5A-5C). The conjugated mAbs were still capable of binding to Jurkat cells (FIGS. 5A-5C). The binding of the conjugated mAbs to Jurkat cells was inhibited by BSA, indicating that the conjugated FA was capable of binding BSA, which subsequently reduced the antigen-binding by the anti-CD3 mAb. To confirm the inhibitory effect of BSA on the CD3 binding by the conjugated mAbs, a T cell activation assay was carried out using peripheral blood mononuclear cells (PBMCs) from two different donors. PBMCs were incubated with the indicated antibodies in media containing multiple concentrations of BSA for 16 hours. T cell activation was assessed by measuring CD25 expression via FACS. Since the media contains 1% FBS, low level of BSA (about 0.25 mg/mL by estimation from the 1% FBS) is in each group in the assay, which is expected to suppress the T cell activation by the conjugated mAbs before BSA was added to the media (FIGS. 6A-6C). When BSA was added to the media, increased inhibition of T cell activation by the added BSA was observed (FIGS. 6A-6C).

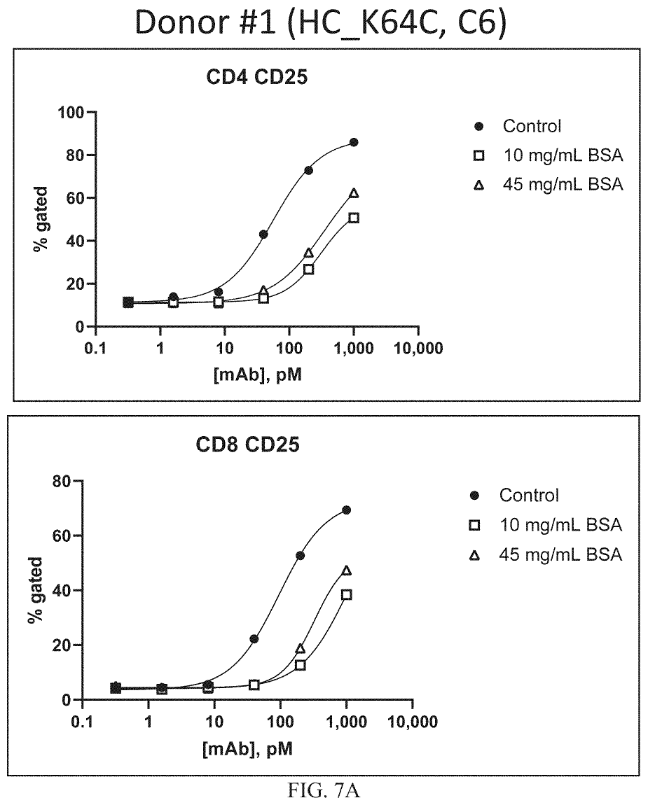

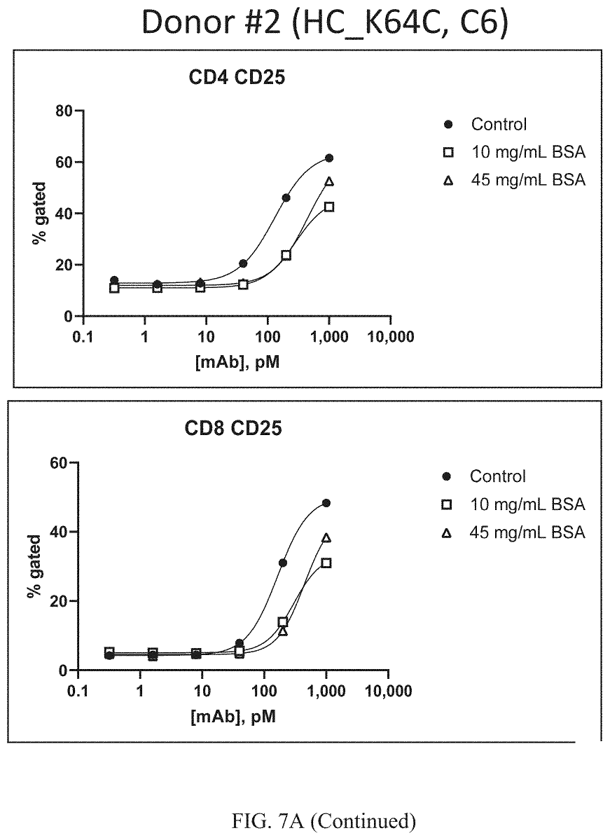

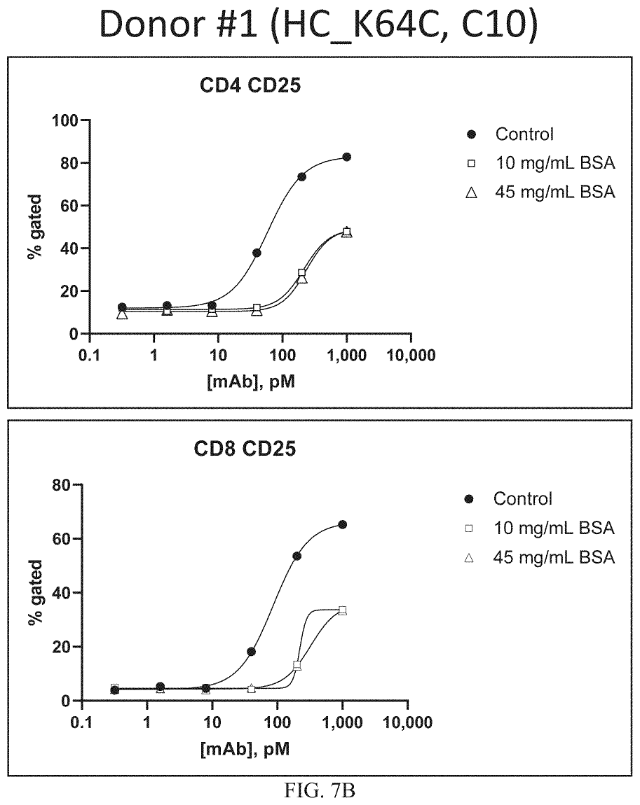

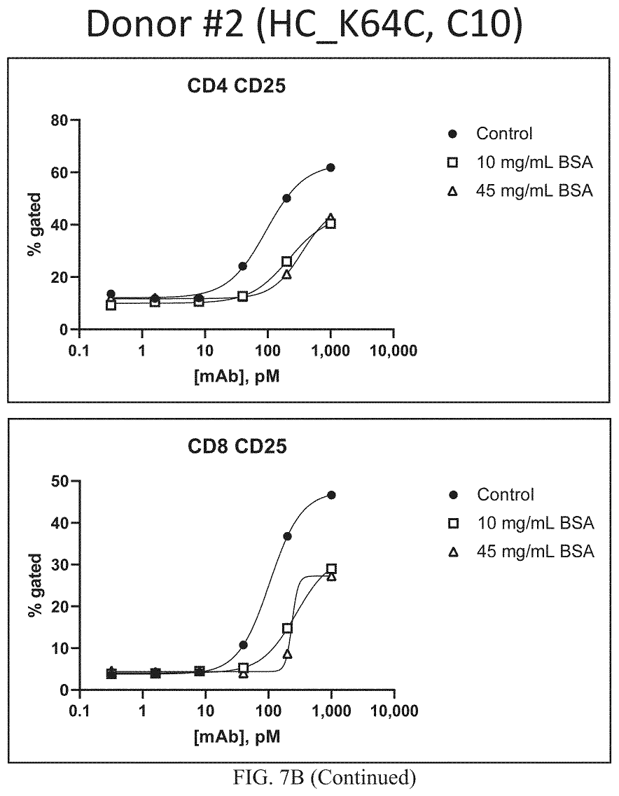

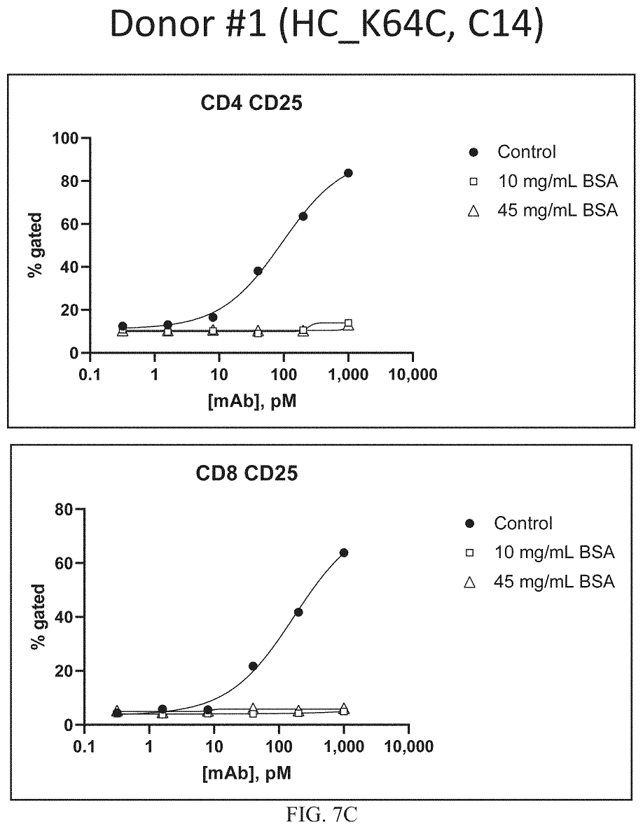

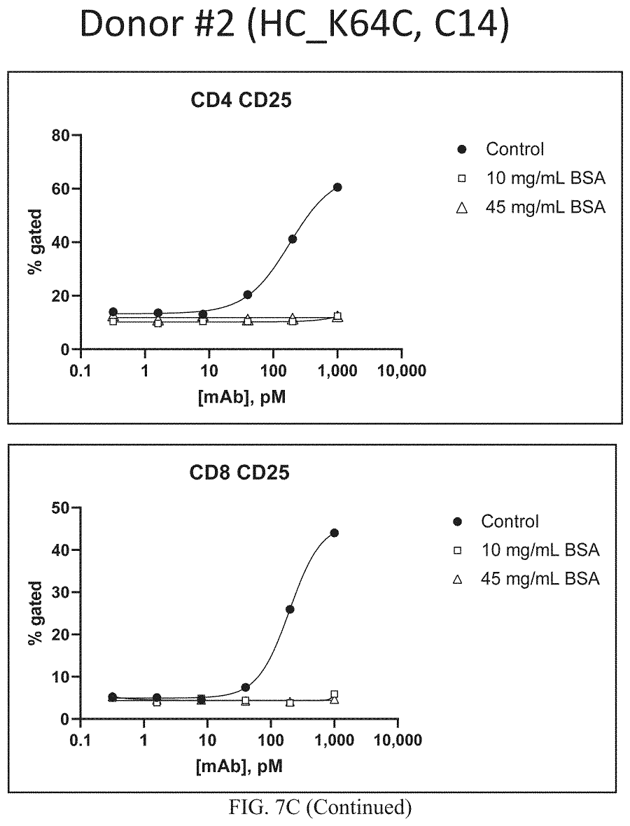

To test the effect of the length of the conjugated FA molecule, C6, C10, and C14 FA molecules were conjugated to HC_K64C, respectively. The conjugation of each of the FA specifically to the heavy chain of each mAb was confirmed by LC/MS (FIG. 4C). The conjugation of the FA to each cysteine knock-in site was confirmed by LC/MS (Table 4). The conjugated mAbs were tested for T cell activation using PBMCs from two different donors as described above (FIGS. 7A-7C). C6 and C10 conjugations were less effective in blocking CD3 binding in the presence of higher concentrations of BSA (FIGS. 7A-7B); C14 conjugation completely blocked CD3 binding in the presence of higher concentrations of BSA (FIG. 7C). These data indicated that longer FA molecules such as C14 and C18, upon conjugation, were more potent in blocking CD3 binding through bound BSA, whereas the shorter C6 and C10 FA molecules, upon conjugation, were less potent in blocking CD3 binding through bound BSA. Each of these characteristics can be leveraged therapeutically under different conditions in the tumor microenvironment. For example, depending on the differential albumin levels between the tumor microenvironment and the circulating blood, a longer FA or a shorter FA could be preferred as the conjugated molecule to achieve the best in vivo efficacy/safety margin.

Characterization of Bispecific Antibodies Conjugated with Fatty Acid Molecules

The FA conjugation approach can be used to modulate the antigen binding activity of one of the two arms of a bispecific antibody. For example, the bispecific antibody can be an anti-TAA/anti-CD3 T cell engager, where the FA is conjugated to the Fab region of the anti-CD3 arm (illustrated as the Ab1 arm in FIGS. 1B-1C). Here an anti-DLL3 arm was used as an example for the anti-TAA arm. The sequences used for constructing the anti-DLL3/anti-CD3 bispecific antibody (as first described in U.S. Provisional Patent Application No. 63/146,334, filed on Feb. 5, 2021, which is incorporated by reference herein in its entirety) were used to introduce cysteine knock-ins for FA conjugation. A cysteine was knocked into the K64 position (VH region; Kabat numbering) or the T120 position (CH1 region; EU numbering) of the anti-CD3 arm of the anti-DLL3/anti-CD3 bispecific antibody. The resulting bsAbs are named bsAb HC_K64C and bsAb HC_T120C, respectively, and their sequences are listed in Table 5. Note that the bsAb HC_K64C and bsAb HC_T120C have the same anti-DLL3 arm (Table 5). The bispecific antibodies are on the human IgG1 HC and human kappa LC framework with the following modifications in the Fc region of IgG1: the HC of the anti-CD3 arm has the T366W (EU numbering) mutation to form a “knob” and the HC of the anti-DLL3 arm has the mutations T366S, L368A, and Y407V to form a “hole.” In addition, a S354C cysteine mutation was introduced on anti-CD3 HC and a Y349C cysteine mutation was introduced on anti-DLL3 HC to stabilize the heterodimeric pairing. Further, L234A and L235A mutations were introduced in the CH2 regions of both H1 and H2.

TABLE 5
Sequences of the VH, VL, CH1, and CL regions of bispecific antibodies
with knock-in cysteine for conjugation

| | | SEQ |
|---|---|---|
| | | ID |
| Region name | Sequence | NO: |
| bsAb HC_K64C, | DIKLQQSGAELARPGASVKMSCKTSGYTFTRYTMHWVKKRPGQGLEW | 15 |
| anti-CD3 VH | IGYINPSRGYTNYNQKFCDKATLTTDKSSSTAYMQLSSLTSEDSAVYYC | |
| | ARYYDDHYSLDYWGQGTTLTVSS | |
| bsAb HC_K64C, | ASTKGPSVFPLAPSSKSTSGGTAALGCLVKDYFPEPVTVSWNSGALTSG | 16 |
| anti-CD3 CHI | VHTFPAVLQSSGLYSLSSVVKVPSSSLGTQTYICNVNHKPSNTKVDKKV | |
| | EPKSC | |
| bsAb HC_K64C, | DIQLTQSPAIMSASPGEKVTMTCRASSSVSYMNWYQEKSGTSPKRWIY | 17 |
| anti-CD3 VL | DTSKVASGVPYRFSGSGSGTSYSLTISSMEAEDAATYYCQQWSSNPLTF | |
| | GAGTKLELK | |
| bsAb HC_K64C, | RTVAAPSVFIFPPSDEQLKSGTASVVCLLNNFYPREAKVQWKVDNALQS | 18 |
| anti-CD3 CL | GNSQESVTEQDSKESTYSLSSTLTLSKADYEKHKVYACEVTHQGLSSPV | |
| | TKSFNRGEC | |
| bsAb HC_T120C, | DIKLQQSGAELARPGASVKMSCKTSGYTFTRYTMHWVKKRPGQGLEW | 19 |
| anti-CD3 VH | IGYINPSRGYTNYNQKFKDKATLTTDKSSSTAYMQLSSLTSEDSAVYYC | |
| | ARYYDDHYSLDYWGQGTTLTVSS | |
| bsAb HC_T120C, | ASCKGPSVFPLAPSSKSTSGGTAALGCLVKDYFPEPVTVSWNSGALTSG | 20 |
| anti-CD3 CH1 | VHTFPAVLQSSGLYSLSSVVKVPSSSLGTQTYICNVNHKPSNTKVDKKV | |
| | EPKSC | |
| bsAb HC_T120C, | DIQLTQSPAIMSASPGEKVTMTCRASSSVSYMNWYQEKSGTSPKRWIY | 21 |
| anti-CD3 VL | DTSKVASGVPYRFSGSGSGTSYSLTISSMEAEDAATYYCQQWSSNPLTF | |
| | GAGTKLELK | |
| bsAb HC_T120C, | RTVAAPSVFIFPPSDEQLKSGTASVVCLLNNFYPREAKVQWKVDNALQS | 22 |
| anti-CD3 CL | GNSQESVTEQDSKESTYSLSSTLTLSKADYEKHKVYACEVTHQGLSSPV | |
| | TKSFNRGEC | |
| Anti-DLL3 VH | EVRLSQSGGQMKKPGESMRLSCRASGYTFTSYVMHWVREAPGRRPEW | 23 |
| | IGYINPYNDATKYARKFQGRATLTSDKYSDTAFLELRSLTSDDTAVYYC | |
| | ARGGYDYDGDYWGRGAPVTVSS | |
| Anti-DLL3 CH1 | ASTKGPSVFPLAPSSCSTSGGTAALGCLVKDYFPEPVTVSWNSGALTSG | 24 |
| | VHTFPAVLQSSGLYSLSSVVEVPSSSLGTQTYICNVNHKPSNTKVDKKV | |
| | EPKSS | |
| Anti-DLL3 VL | EIVLTQSPGTLSLSPGERATLSCHASQNINVWLSWYQKKPGQAPRLLIY | 25 |
| | KASNLHTGIPDRFSGSGSGTDFTLTISRLEPEDFAVYYCQQGQSYPFTFG | |
| | QGTKVEIK | |
| Anti-DLL3 CL | RTVAAPSVFIFPPSDEQLKSGTASVVCLLNNFYPREAKVQWKVDNALQS | 26 |
| | GNSQESVTEQDSKKSTYSLSSTLTLSKADYEKHKVYACEVTHQGLSSPV | |
| | TKSCNRGES | |
| bsAb HC_K64C, | QVQLQQSGAELARPGASVKMSCKASGYTFTRYTMHWVKKRPGQGLE | 29 |
| #2 anti-CD3 VH | WIGYINPSRGYTNYNQKFCDKATLTTDKSSSTAYMQLSSLTSEDSAVYY | |
| | C ARYYDDHYSLDYWGQGTTLTVSS | |
| bsAb HC_K64C, | QIVLTQSPAIMSASPGEKVTMTCSASSSVSYMNWYQEKSGTSPKRWIYD | 30 |
| #2 anti-CD3 VL | TSKLASGVPAHFRGSGSGTSYSLTISGMEAEDAATYYCQQWSSNPFTFG | |
| | SGTKLEIN | |
| bsAb HC_T120C, | QVQLQQSGAELARPGASVKMSCKASGYTFTRYTMHWVKKRPGQGLE | 31 |
| #2 anti-CD3 VH | WIGYINPSRGYTNYNQKFKDKATLTTDKSSSTAYMQLSSLTSEDSAVYY | |
| | CARYYDDHYSLDYWGQGTTLTVSS | |
| bsAb HC_T120C, | QIVLTQSPAIMSASPGEKVTMTCSASSSVSYMNWYQEKSGTSPKRWIYD | 32 |
| #2 anti-CD3 VL | TSKLASGVPAHFRGSGSGTSYSLTISGMEAEDAATYYCQQWSSNPFTFG | |
| | SGTKLEIN | |

Note:
VH and VL, Kabat numbering; CH1 and CL, EU numbering

The bsAb HC_K64C and bsAb HC_T120C bispecific antibodies were transiently transfected in ExpiCHO-S cells, and the simultaneous expression of the two heavy chains and the two light chains in the same cell led to the expression and assembly of a desired bispecific antibody and certain impurities. The impurity standards were made by transient transfection using the same HC and LC vectors as needed. The bispecific antibodies were purified first using Protein A chromatography. Protein A purified samples were pH adjusted to a final pH of 5.5 and loaded directly onto a poros XS (Thermo) CEX column pre-equilibrated with 25 mM phosphate (pH 5.8)+210 mM NaCl. Samples were eluted with a linear gradient [Buffer A—25 mM phosphate (pH 5.8)+210 mM NaCl; Buffer B—25 mM phosphate (pH 8)+115 mM NaCl]. Eluted fractions were analyzed by strong cation exchange (SCX) HPLC, and fractions showing the complete elimination of 2× anti-DLL3 LC mismatch (the HC heterodimer with the anti-DLL3 LC matched on both arms) were pooled. (NH₄)₂SO₄ was added to pooled fractions to a final concentration of 700 mM, and the sample was loaded directly onto a Butyl Sepharose High Performance (Cytiva) HIC (hydrophobic interaction chromatography) column pre-equilibrated with 50 mM tris (pH 7.5)+700 mM (NH₄)₂SO₄+3% glycerol. Samples were eluted using a linear gradient [Buffer A—50 mM tris (pH 7.5)+700 mM (NH₄)₂SO₄+3% glycerol; Buffer B—50 mM tris (pH 7.5)+10% glycerol]. Eluted fractions were analyzed by HIC HPLC, and fractions showing the complete elimination of 2× anti-CD3 LC mismatch (the HC heterodimer with the anti-CD3 LC matched on both arms) were pooled as purified protein. The purified bispecific antibodies were analyzed with three different methods.

For HIC HPLC, samples were diluted to a final concentration of 1 mg/mL in buffer containing 1 M (NH₄)₂SO₄, and 15 μl was injected directly for HIC HPLC analysis using an Agilent AdvanceBio HIC 4.6×100 mm 3.6 μm column (PN: 685975-908). Samples were analyzed at a flow rate of 1 mL/min at 30° C. using a linear gradient (Buffer A—50 mM Tris pH 7.5+1 M (NH₄)₂SO₄; Buffer B—50 mM Tris pH 7.5+10% glycerol).

For SCX HPLC, samples were diluted to a final concentration of 1 mg/mL in buffer containing 25 mM citrate pH 4.5, and 15 μl was injected directly for SCX HPLC analysis using a Waters Bioresolve SCX mAb 4.6×100 mm 3 μm column (PN: 18609060). Samples were analyzed at a flow rate of 1 mL/min at 30° C. using a linear gradient (Buffer A—25 mM phosphate pH 5.8+2% ACN; Buffer B—25 mM phosphate pH 8+250 mM NaCl+2% ACN).

For size-exclusion chromatography (SEC) HPLC, samples were diluted to a final concentration of 1 mg/mL in PBS, and 8 μl was injected directly for SEC HPLC analysis using an Agilent AdvanceBio SEC 300A 2.7 μm 4.6×300 mm column (PN: PL1580-5301). Samples were analyzed at a flow rate of 0.35 mL/min using an isocratic elution (Buffer—50 mM phosphate pH 7.4+300 mM NaCl+5% isopropanol).

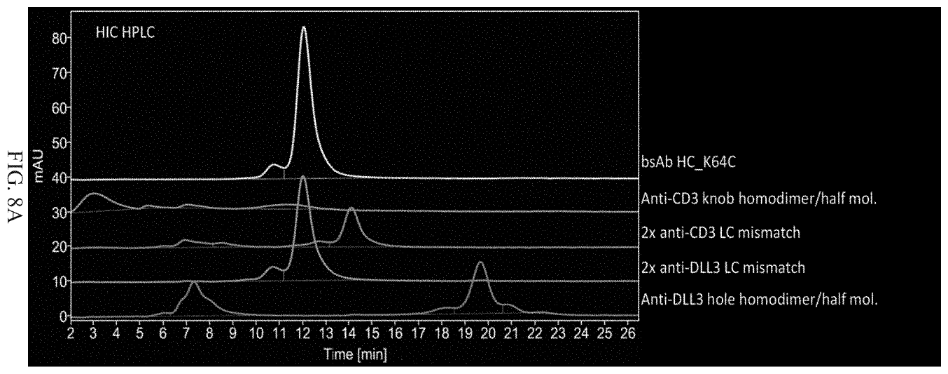

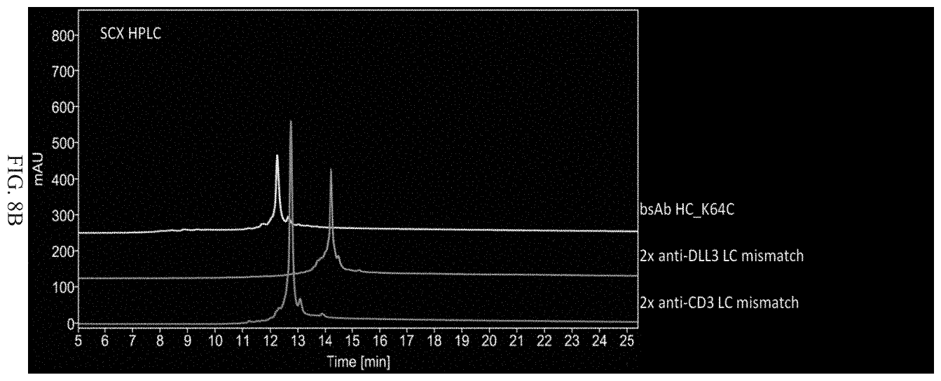

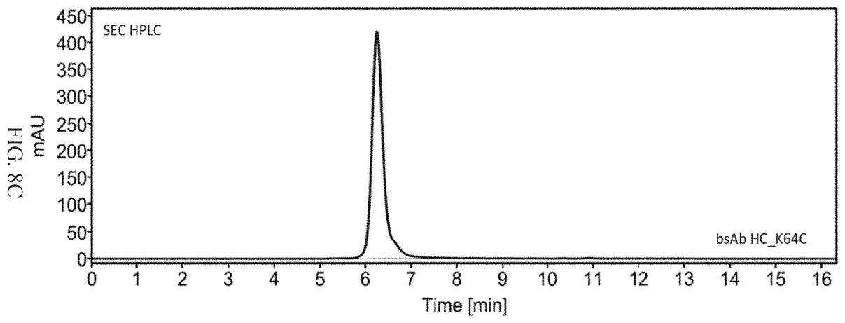

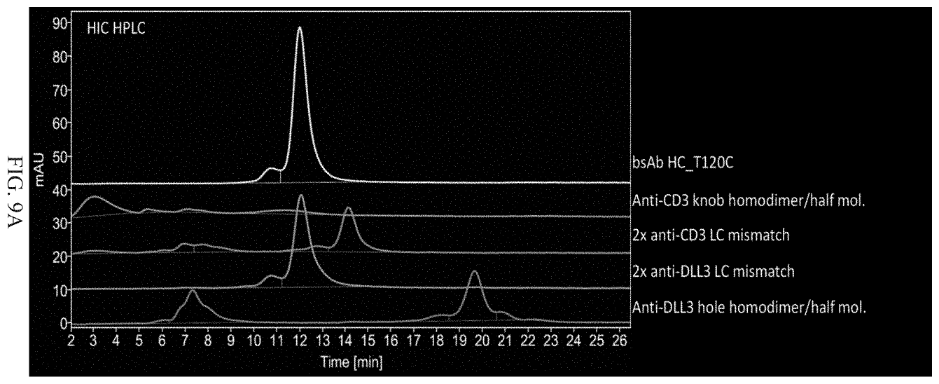

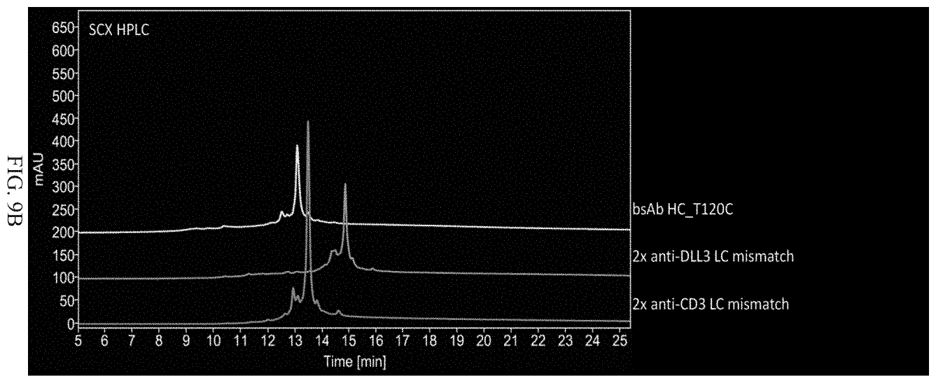

The purified bsAbs were analyzed on HIC HPLC, SCX HPLC and SEC HPLC (FIGS. 8A-8B and 9A-9B). In FIG. 8A, the purified bsAb HC_K64C is separated from the impurities except the 2× anti-DLL3 LC mismatch on HIC HPLC; however, when analyzed on SCX HPLC, the purified bsAb HC_K64C was well separated from the 2× anti-DLL3 LC mismatch (FIG. 8B). These data demonstrate that the purified bsAb HC_K64C was free of the impurities. Further, the purified bsAb HC_K64C was a single species on SEC HPLC (FIG. 8C). Similar observations were made with bsAb HC_T120C (FIGS. 9A-9B), indicating the high purity of the purified bsAb HC_T120C.

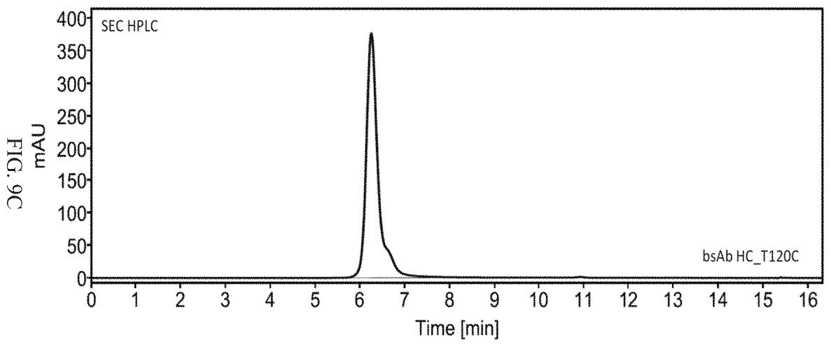

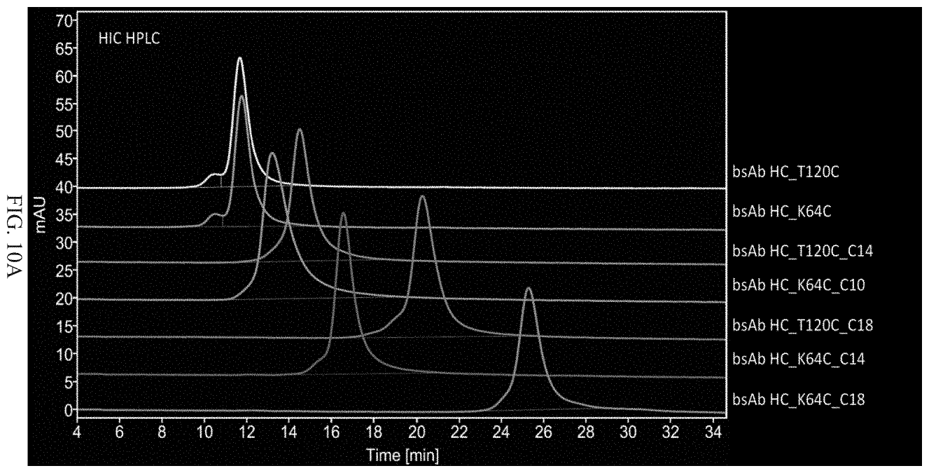

The purified bsAb HC_K64C and bsAb HC_T120C bispecific antibodies were conjugated with different FA molecules. For conjugation, a bispecific antibody with a knocked in cysteine at K64 or T120 was concentrated to a concentration of 20-30 mg/mL and buffer exchanged into TBS buffer. The bispecific antibody was partially reduced with the addition of 10 equivalents of TCEP, and the solution was incubated for 1 hour at 37° C. The bispecific antibody was then buffer exchanged into DPBS and re-oxidized with 30 equivalents of dehydroascorbic acid added, and the solution was incubated for 1 hour at RT. The final bispecific antibody sample was buffer exchanged into conjugation buffer (20 mM Tris pH 8.5+150 mM NaCl+10% glycerol) and diluted to a concentration of 10 mg/mL. A FA molecule was added at 12 equivalents from a 50 mM solution in DMSO, and the resulting mixture was incubated at RT for 1 day. The final product was buffer exchanged into conjugation buffer to remove unreacted FA molecules, and then purified by HIC purification. The purified conjugated bispecific antibodies were analyzed on HIC HPLC (FIG. 10A) and SEC HPLC (FIG. 10B). Each of the conjugated bsAbs appeared as a single peak with a retention time different from that of the corresponding unconjugated bsAb (FIG. 10A), demonstrating high efficiency of conjugation. Further, each conjugated bsAb appeared as a single peak on SEC HPLC (FIG. 10B).

TABLE 6
Confirmation of the conjugation of a FA to the bsAbs

| | Calculated | Observed | |
|---|---|---|---|
| bsAb | mw | mw | Notes |
| Parental bsAb | 145307.4 | 145310.7 | Expected |
| bsAb HC_K64C | 145282.4 | 145404.8 | +1 cysteine |
| bsAb HC_T120C | 145309.4 | 145433.0 | +1 cysteine |
| bsAb HC_K64C_C10 | 145963.3 | 145967.4 | Expected |
| bsAb HC_K64C_C14 | 146019.4 | 146022.5 | Expected |
| bsAb HC_K64C_C18 | 146075.5 | 146079.1 | Expected |
| bsAb HC_T120C_C14 | 146046.4 | 146051.2 | Expected |
| bsAb HC_T120C_C18 | 146102.5 | 146106.6 | Expected |

Note:
Parental bsAb, the anti-DLL3/anti-CD3 bsAb without cysteine knock-in; mw, molecular weight; +1 cysteine, one cysteine is expected to be covalently linked to the knocked in cysteine and the resulting mw is as expected.

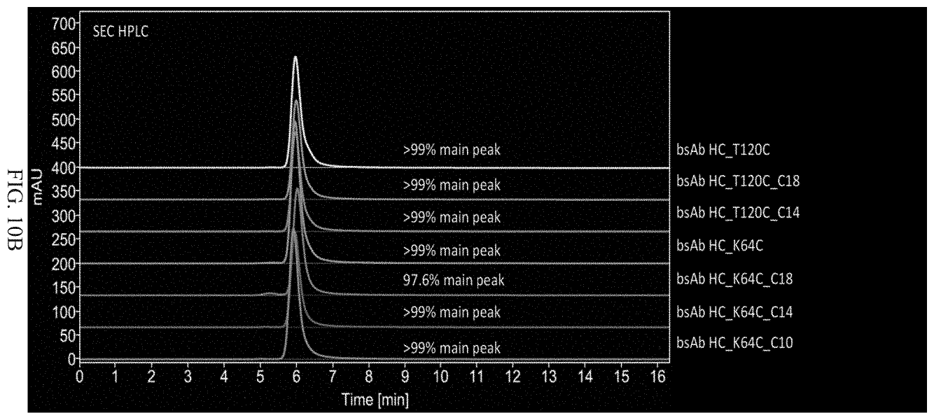

To assess the binding activities of the unconjugated and conjugated bispecific antibodies to both DLL3 and CD3 at the same time, the purified bispecific antibodies were incubated with SHP-77 cells and Jurkat cells, which were labeled with different fluorescent markers. The double-stained events induced by a bispecific antibody were detected and quantified by flow cytometry. Briefly, Jurkat cells were stained with Violet Proliferation Dye 450 (BD, Cat: 562158) and SHP-77 cells were stained by CFSE (ThermoFisher, Cat: 34554) according to the manufacturer's protocol. The labelled SHP-77 and Jurkat cells at 1:1 ratio were then incubated with 2 μg/mL bsAb in the presence or absence of 1.5 μM anti-DLL3 blocking mAb or 1.5 μM anti-CD3 blocking mAb (FIG. 11). When blocking mAb was used, SHP-77 cells were pretreated with 4.5 μM anti-DLL3 blocking mAb for 10 minutes at room temperature before incubation with Jurkat cells at the final concentration of 1.5 μM anti-DLL3 blocking mAb, or Jurkat cells were pretreated with 4.5 μM anti-CD3 blocking mAb for 10 minutes at room temperature before incubation with SHP-77 cells at the final concentration of 1.5 μM anti-CD3 blocking mAb. Following incubation in a CO₂ incubator at 37° C. for 1 hour, the cells were fixed with 2% formaldehyde, washed once with TBS, resuspended in FACS buffer (HBSS, 0.1% BSA, 0.05% sodium azide), and then analyzed by flow cytometry (Attune NxT). The cross-linking of the two cell types in the presence of a bsAb was detected on FACS and the signal for each bsAb (unconjugated and conjugated) was inhibited by the anti-DLL3 or the anti-CD3 blocking mAb (FIG. 11). These data demonstrate that the unconjugated and conjugated bsAbs can bind to the two different antigens at the same time.

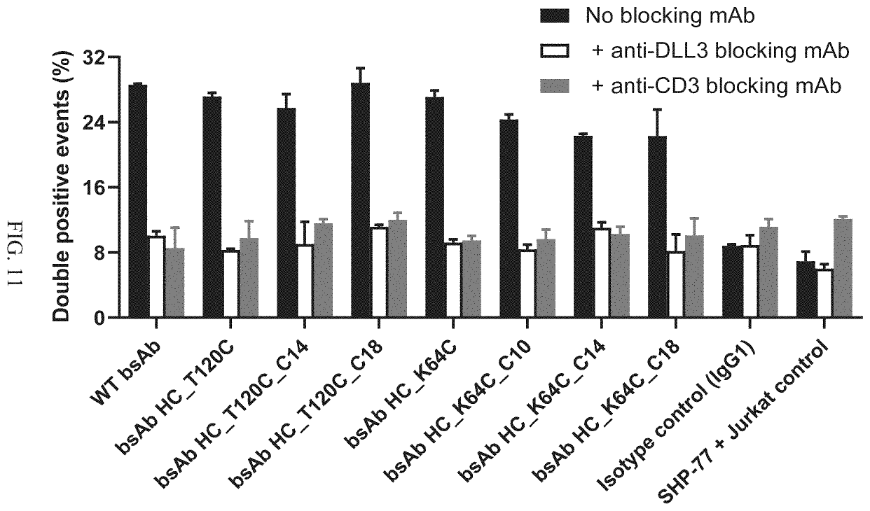

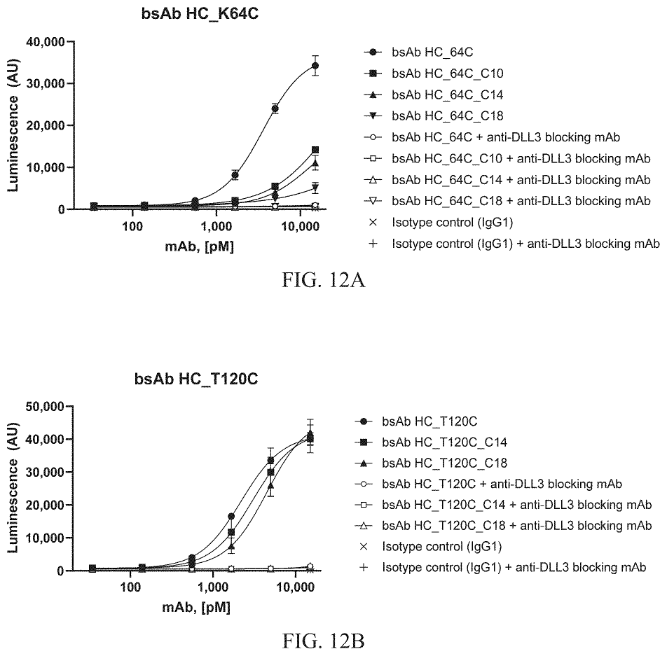

The bispecific antibodies were also used to activate T cells in a functional T cell activation assay. A Jurkat NFAT Luciferase reporter cell line (BPS Bioscience) which conditionally expresses firefly luciferase upon activation (including CD3-mediated activation) was used. The reporter cells were incubated with SHP-77 target cells in the presence of each bsAb (unconjugated and conjugated) and with or without the presence of anti-DLL3 blocking antibody (at the final concentration of 500 nM) for 22 hours in growth media at 37° C. in a CO₂ incubator. The cells were then assayed for activation by a luciferase detection reagent and luminometer. Each of the bispecific antibodies induced dose-dependent activation of the reporter cells when incubated with the target cell SHP-77, and the signal was inhibited by the anti-DLL3 blocking antibody (the mAb version of the anti-DLL3 arm) (FIGS. 12A-12B), demonstrating the T cell engager function of the bispecific antibodies. Since 0.5% FBS (fetal bovine serum) is required as part of the culture media for the cell survival during the assay, low level of BSA (about 0.13 mg/mL by estimation from the 0.5% FBS) is in the assay, which is expected to lead to the reduced T cell activation signal by bsAb HC_K64C_C10, bsAb HC_K64C_C14 and bsAb HC_K64C_C18 (FIG. 12A).

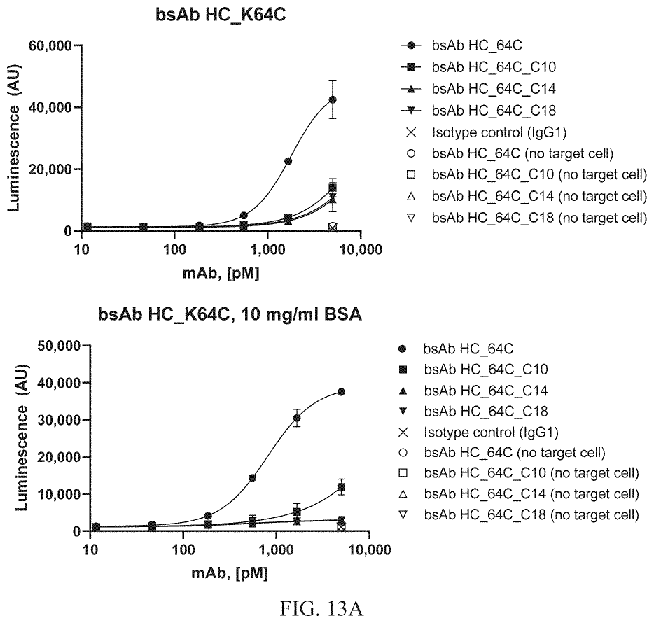

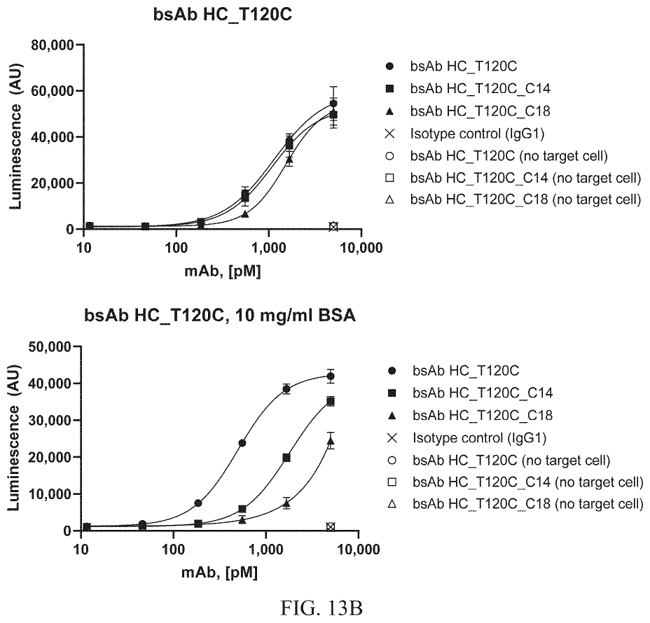

The T cell activation assay using Jurkat NFAT Luciferase reporter cell line was also carried out in the presence of low and high BSA levels to assess the inhibitory effect of albumin on the T cell activation function of the conjugated bispecific antibodies. Since 0.5% FBS is required as part of the culture media for the cell survival during the assay, low level of BSA (about 0.13 mg/mL by estimation from the 0.5% FBS) was in each group in the assay. When high level of BSA (final concentration of 10 mg/mL BSA in addition to the 0.5% FBS) was added to the assay, the T cell activation induced by the conjugated bispecific bsAb HC_K64C_C14 and bsAb HC_K64C_C18 was inhibited when compared with the control group (0.5% FBS only) (FIG. 13A); high level of BSA (10 mg/mL BSA+0.5% FBS) inhibited the T cell activation induced by the conjugated bispecific bsAb HC_T120C_C14 and bsAb HC_T120C_C18 when compared with the control group (0.5% FBS only) (FIG. 13B). These data indicate that the activity of the anti-DLL3/anti-CD3 bispecific antibodies conjugated with FA molecules can be modulated by BSA levels. FIG. 5B indicates that FA conjugation to the mAb HC_K64C did not change the binding of the conjugated arm to CD3; further, FIG. 11 indicates that FA conjugation to the bsAb HC_K64C did not drastically change its bispecific activity, suggesting that the lower activity of bsAb HC_K64C_C10 in FIG. 13A is because of low BSA level carried over by the 0.5% FBS that is required as part of the culture media. Similar observations were made for HC_K64C_C14 and HC_K64C_C18 (FIG. 13A). These observations are consistent with the data in FIGS. 6B and 7A-7C, which indicate that the FA conjugation at K64 impacts the binding of the anti-CD3 arm to CD3 much more than at T120 in the presence of BSA. This is also consistent with the fact that the BSA bound to the FA conjugated at K64C is closer to the CDRs and can more efficiently block the target antigen (CD3) binding than at T120C.

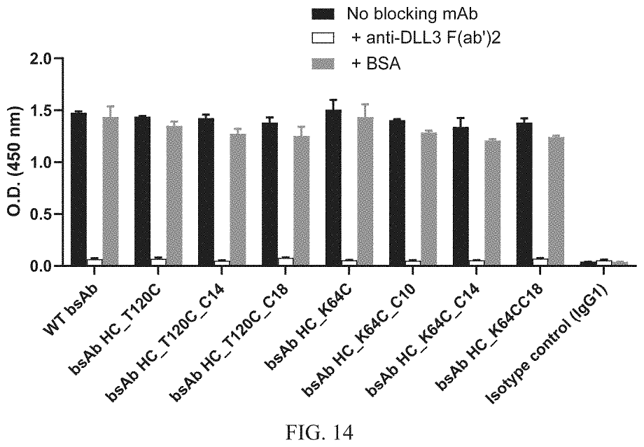

An ELISA assay was carried out to assess the effect of BSA on the antigen-binding activity of the anti-DLL3 arm of each conjugated bispecific antibody. A 96-well ELISA plate was coated with DLL3 protein (Adipogen, Cat #: AG-40B-0151-0010) for 1 hour at RT, followed by blocking with 5% BSA in TBST for 1 hour at RT. The plate was washed 3 times with TBST and pre-incubated at RT for 1 hour with or without blockers (200 μg/mL anti-DLL3 F(ab′)2 or 50 mg/mL BSA (Sigma, Cat #: A4612-25G); TBST was used for the no-blocker groups). Then the plate was incubated with 1 μg/mL bsAb for 30 minutes at RT in the presence or absence of 100 μg/mL anti-DLL3 F(ab′)2 or 50 mg/mL BSA. After incubation, the plate was washed and the signal was detected with HRP conjugated anti-human IgG secondary antibody (ThermoFisher, Cat #: H10007) and TMB substrate (ThermoFisher, Cat #: 34029) using an Envision spectrophotometer. FIG. 14 shows that BSA had very little effect on the antigen-binding activity of the anti-DLL3 arm of each conjugated bsAb.

A second set of anti-CD3 (#2 anti-CD3) VH and VL sequences (SEQ ID NOs:27 and 28, respectively; Kabat numbering) can also be used for constructing the conjugated mAbs and bsAbs and are listed in Table 1. Further, modified versions of these sequences can also be used to construct conjugated anti-DLL3/anti-CD3 bispecific antibodies (Table 5), comprising a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18; or a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22. In each of the above cases, the second antigen-binding arm (Ab2) comprises a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26. Further, each of the first antigen-binding arm (Ab1) mentioned above can be used to construct bispecific antibodies against CD3 and TAAs other than DLL3.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated monoclonal antibody or antigen-binding fragment thereof, wherein the antibody or antigen-binding fragment thereof comprises:
a. a variable heavy chain region (VH);
b. a variable light chain region (VL);
wherein the antibody or antigen-binding fragment thereof binds to a target antigen;
wherein an amino acid residue in the VH, VL, or within a twenty (20)-amino acid distance of the VH or VL on one or both arms is substituted with an amino acid residue that is conjugated to a fatty acid (FA);
and wherein upon conjugation with the FA at the substituted amino acid residue, the monoclonal antibody or antigen-binding fragment thereof still binds to the target antigen.

2. The isolated monoclonal antibody or antigen-binding fragment thereof of claim 1, wherein the substituted amino acid residue thereof is within a five (5)-amino acid distance of the VH or VL on one or both arms.

3. The isolated monoclonal antibody or antigen-binding fragment thereof of claim 1, wherein the substituted amino acid residue thereof is a cysteine residue, a lysine residue, or a modified amino acid that is suitable for chemical conjugation.

4. The isolated monoclonal antibody or antigen-binding fragment thereof of claim 3, wherein the substituted amino acid residue occurs at an amino acid residue corresponding to:
(1) residue 25, 27, 62, 64, 73, 76, 101, 112, or 113 of SEQ ID NO:1;
(2) residue 26, 27, 52, 53, 56, or 67 of SEQ ID NO:2;
(3) residue 119 or 120 of SEQ ID NO:9, 10, 11, or 12; or
(4) residue 121 or 124 of SEQ ID NO:13 or 14.

5. The isolated monoclonal antibody or antigen-binding fragment thereof of claim 4, wherein the substituted amino acid residue occurs at an amino acid residue corresponding to:
(1) a K64C substitution of SEQ ID NO:1;
(2) a S26C substitution of SEQ ID NO:2; or
(3) a T120C substitution of SEQ ID NO:9, 10, 11, or 12.

6. The isolated monoclonal antibody or antigen-binding fragment thereof of claim 1, wherein the monoclonal antibody or antigen-binding fragment thereof is an anti-immune cell modulator (ICM) antibody or antigen-binding fragment thereof and capable of specific binding to the ICM.

7. The isolated monoclonal antibody or antigen-binding fragment thereof of claim 6, wherein the ICM is selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory antigens.

8. The isolated monoclonal antibody or antigen-binding fragment thereof of claim 7, wherein the ICM is CD3, and wherein the monoclonal antibody or antigen-binding fragment thereof comprise a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:3, 4, 5, 6, 7, and 8, respectively; or SEQ ID NOs:33, 34, 35, 36, 37, and 38, respectively.

9. The isolated monoclonal antibody or antigen-binding fragment thereof of claim 8, wherein the substituted amino acid residue occurs at an amino acid residue selected from:
(1) residue 25, 27, 62, 64, 73, 76, 101, 112, or 113 of SEQ ID NO:1 or 27;
(2) residue 26, 27, 52, 53, 56, or 67 of SEQ ID NO:2 or 28;
(3) residue 119 or 120 of SEQ ID NO: 9, 10, 11, or 12; or
(4) residue 121 or 124 of SEQ ID NO: 13 or 14.

10. The isolated monoclonal antibody or antigen-binding fragment thereof of claim 5, comprising:
1) a VH region having a polypeptide sequence of SEQ ID NO:1 with an amino acid substitution of K64C and a VL region having a polypeptide sequence of SEQ ID NO:2;
2) a VH region having a polypeptide sequence of SEQ ID NO:27 with an amino acid substitution of K64C and a VL region having a polypeptide sequence of SEQ ID NO:28;
3) a VH region having a polypeptide sequence of SEQ ID NO:1 and a VL region having a polypeptide sequence of SEQ ID NO:2 with an amino acid substitution of S26C;
4) a VH region having a polypeptide sequence of SEQ ID NO:27 and a VL region having a polypeptide sequence of SEQ ID NO:28 with an amino acid substitution of S26C;
5) a CH1 region having a polypeptide sequence selected from SEQ ID NO: 9, 10, 11 or 12 with an amino acid substitution of T120C and a CL region having a polypeptide sequence selected from SEQ ID NO:13 or 14;
6) a VH region having a polypeptide sequence of SEQ ID NO:1, a VL region having a polypeptide sequence of SEQ ID NO:2, a CH1 region having a polypeptide sequence selected from SEQ ID NO: 9, 10, 11 or 12 with an amino acid substitution of T120C, and a CL region having a polypeptide sequence selected from SEQ ID NO:13 or 14;
7) a VH region having a polypeptide sequence of SEQ ID NO:27, a VL region having a polypeptide sequence of SEQ ID NO:28, a CH1 region having a polypeptide sequence selected from SEQ ID NO: 9, 10, 11 or 12 with an amino acid substitution of T120C, and a CL region having a polypeptide sequence selected from SEQ ID NO:13 or 14.

11. An isolated multi-specific antibody or antigen-binding fragment thereof, wherein the multi-specific antibody or antigen-binding fragment thereof comprises the monoclonal antibody or antigen-binding fragment thereof of claim 1, and wherein the multi-specific antibody or antigen-binding fragment thereof comprises one or more antigen-binding arm(s) comprising a substituted amino acid residue that is conjugated to a FA.

12. The isolated multi-specific antibody or antigen-binding fragment thereof of claim 11, wherein the multi-specific antibody or antigen-binding fragment thereof is a bispecific antibody or antigen-binding fragment comprising a first antigen-binding arm (Ab1) and a second antigen-binding arm (Ab2), wherein Ab1 and/or Ab2 comprise a substituted amino acid that is conjugated to a FA.

13. The isolated bispecific antibody or antigen-binding fragment thereof of claim 12, wherein Ab1 binds an immune cell modulator (ICM).

14. The isolated bispecific antibody or antigen-binding fragment thereof of claim 13, wherein the ICM is selected from the group consisting of CD3, CD27, CD28, CD40, CD122, OX40, CD16, 4-1BB, GITR, ICOS, CTLA-4, PD-1, LAG-3, TIM-3, TIGIT, VISTA, SIGLEC7, NKG2D, SIGLEC9, KIR, CD91, BTLA, NKp46, B7-H3, SIPRα, and other cell surface immune regulatory molecules.

15. The isolated bispecific antibody or antigen-binding fragment thereof of claim 12, wherein Ab2 binds a tumor-associated antigen (TAA).

16. The isolated bispecific antibody or antigen-binding fragment thereof of claim 15, wherein the TAA is DLL3.

17. The isolated bispecific antibody or antigen-binding fragment thereof claim 12, wherein the first antigen-binding arm (Ab1) comprises H1 and L1 and a second antigen-binding arm (Ab2) comprises H2 and L2, wherein
(a) H1 and H2 each comprises a CH1 region of human IgG1, IgG2, IgG3, or IgG4; and
(b) L1 and L2 each comprises a CL region of a human kappa light chain or a human lambda light chain;
wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) K/R133D/E in CH1 of H1 and K207K/R in CL of L1, respectively, and K/R133K/R in CH1 of H2 and K207D/E in CL of L2, respectively;
(6) K/R133D/E in CH1 of H1 and I/L117K/R in CL of L1, respectively, and K/R133K/R in CH1 of H2 and I/L117D/E in CL of L2, respectively;
(7) K/R133D/E in CH1 of H1 and F/V209K/R in CL of L1, respectively, and K/R133K/R in CH1 of H2 and F/V209D/E in CL of L2, respectively;
(8) G166D/E in CH1 of H2 and S114K/R in CL of L2, respectively, and G166K/R in CH1 of H1 and S114D/E in CL of L1, respectively;
(9) T187D/E in CH1 of H2 and D/N170K/R in CL of L2, respectively, and T187K/R in CH1 of H1 and D/N170D/E in CL of L1, respectively;
(10) S131D/E in CH1 of H2 and P119K/R in CL of L2, respectively, and S131K/R in CH1 of H1 and P119D/E in CL of L1, respectively;
(11) A129D/E in CH1 of H2 and S121K/R in CL of L2, respectively, and A129K/R in CH1 of H1 and S121D/E in CL of L1, respectively;
(12) K/R133D/E in CH1 of H2 and K207K/R in CL of L2, respectively, and K/R133K/R in CH1 of H1 and K207D/E in CL of L1, respectively;
(13) K/R133D/E in CH1 of H2 and I/L117K/R in CL of L2, respectively, and K/R133K/R in CH1 of H1 and I/L117D/E in CL of L1, respectively; or
(14) K/R133D/E in CH1 of H2 and F/V209K/R in CL of L2, respectively, and K/R133K/R in CH1 of H1 and F/V209D/E in CL of L1, respectively.

18. The isolated bispecific antibody or antigen-binding fragment thereof of claim 12, comprising:
1) a first antigen-binding arm (Ab1) comprising a VH region having a polypeptide sequence of SEQ ID NO:15, a VL region having a polypeptide sequence of SEQ ID NO:17, a CH1 region having a polypeptide sequence of SEQ ID NO:16, and a CL region having a polypeptide sequence of SEQ ID NO:18;
2) a first antigen-binding arm (Ab1) comprising a VH region having a polypeptide sequence of SEQ ID NO:19, a VL region having a polypeptide sequence of SEQ ID NO:21, a CH1 region having a polypeptide sequence of SEQ ID NO:20, and a CL region having a polypeptide sequence of SEQ ID NO:22;
3) a first antigen-binding arm (Ab1) comprising a VH region having a polypeptide sequence of SEQ ID NO:29, a VL region having a polypeptide sequence of SEQ ID NO:30, a CH1 region having a polypeptide sequence of SEQ ID NO:16, and a CL region having a polypeptide sequence of SEQ ID NO:18; or
4) a first antigen-binding arm (Ab1) comprising a VH region having a polypeptide sequence of SEQ ID NO:31, a VL region having a polypeptide sequence of SEQ ID NO:32, a CH1 region having a polypeptide sequence of SEQ ID NO:20, and a CL region having a polypeptide sequence of SEQ ID NO:22.

19. The isolated bispecific antibody or antigen-binding fragment thereof of claim 18, wherein the second antigen-binding arm (Ab2) comprises a VH region having a polypeptide sequence of SEQ ID NO:23, a VL region having a polypeptide sequence of SEQ ID NO:25, a CH1 region having a polypeptide sequence of SEQ ID NO:24, and a CL region having a polypeptide sequence of SEQ ID NO:26.

20. The isolated antibody or antigen-binding fragment thereof of claim 1, wherein the FA is selected from a FA with 6 carbons, 8 carbons, 10 carbons, 12 carbons, 14 carbons, 16 carbons, or 18 carbons, or any number of carbons in between.

21. (canceled)

22. The isolated antibody or antigen-binding fragment thereof of claim 1, wherein the FA comprises a linker for conjugation to the substituted amino acid residue, wherein the linker is selected from a peptide linker or a polyethylene glycol linker.

23.-24. (canceled)

25. The isolated antibody or antigen-binding fragment thereof of claim 1, wherein the FA conjugated to the antibody or antigen-binding fragment thereof is capable of binding albumin, wherein the binding of albumin to the FA results in (a) a partial or a complete blocking of the binding between the target antigen and the antibody or antigen-binding fragment thereof; and/or reduced ability to activate T cells upon binding to albumin as compared to the isolated antibody or antigen-binding fragment thereof not binding to albumin.

26. (canceled)

27. An isolated nucleic acid encoding the isolated antibody or antigen-binding fragment thereof of claim 1.

28. A vector comprising the isolated nucleic acid of claim 27.

29. An isolated host cell comprising the vector of claim 28.

30. A pharmaceutical composition comprising the isolated antibody or antigen-binding fragment thereof claim 1, and a pharmaceutically acceptable carrier.

31. A method of treating a cancer in a subject in need thereof, the method comprising administering to the subject the pharmaceutical composition of claim 30.

32. The method of claim 31, wherein the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

33. A method of producing the isolated antibody or antigen-binding fragment thereof of claim 1, the method comprising culturing a cell comprising a nucleic acid encoding the antibody or antigen-binding fragment thereof under conditions to produce the antibody or antigen-binding fragment thereof, and recovering the antibody or antigen-binding fragment thereof from the cell or culture.

34. (canceled)

35. A method of producing a pharmaceutical composition comprising the isolated antibody or antigen-binding fragment thereof of claim 1, the method comprising combining the antibody or antigen-binding fragment thereof with a pharmaceutically acceptable carrier to obtain the pharmaceutical composition.

36. A method, comprising contacting albumin with the isolated antibody or antigen-binding fragment thereof of claim 1, wherein the antibody or antigen-binding fragment thereof is capable of specific binding to a target antigen, the FA is capable of binding to albumin, and the binding of albumin to the FA results in a partial or a complete blocking of the binding between the target antigen and the antibody or antigen-binding fragment thereof.

37. The method of claim 36, wherein the contacting step comprises administering a pharmaceutical composition comprising the isolated antibody or antigen-binding fragment thereof to a subject in need of a treatment of a tumor, wherein the tumor comprises the target antigen.

38. The method of claim 36, wherein albumin has a higher turnover rate in the tumor microenvironment compared with the circulating blood, and/or is present in the tumor microenvironment at a level lower than the albumin level in circulating blood of the subject.


SEQUENCE LISTING (38 sequences)

SEQ ID NO: 1 - protein, 119 aa, Artificial Sequence
  Anti-CD3 variable heavy chain region (VH)
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 2 - protein, 106 aa, Artificial Sequence
  Anti-CD3 variable light chain region (VL)
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 3 - protein, 10 aa, Artificial Sequence
  Anti-CD3 HCDR1
GYTFTRYTMH

SEQ ID NO: 4 - protein, 17 aa, Artificial Sequence
  Anti-CD3 HCDR2
YINPSRGYTNYNQKFKD

SEQ ID NO: 5 - protein, 12 aa, Artificial Sequence
  Anti-CD3 HCDR3
ARYYDDHYSLDY

SEQ ID NO: 6 - protein, 10 aa, Artificial Sequence
  Anti-CD3 LCDR1
RASSSVSYMN

SEQ ID NO: 7 - protein, 7 aa, Artificial Sequence
  Anti-CD3 LCDR2
DTSKVAS

SEQ ID NO: 8 - protein, 9 aa, Artificial Sequence
  Anti-CD3 LCDR3
QQWSSNPLT

SEQ ID NO: 9 - protein, 103 aa, Artificial Sequence
  IgG1 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 103 aa, Artificial Sequence
  IgG2 CH1
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCC

SEQ ID NO: 11 - protein, 105 aa, Artificial Sequence
  IgG3 CH1
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLG

SEQ ID NO: 12 - protein, 103 aa, Artificial Sequence
  IgG4 CH1
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYG

SEQ ID NO: 13 - protein, 107 aa, Artificial Sequence [sequence omitted]

SEQ ID NO: 14 - protein, 104 aa, Artificial Sequence
  Lambda CL
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 15 - protein, 119 aa, Artificial Sequence
  bsAb HC_K64C, anti-CD3 VH
DIKLQQSGAELARPGASVKMSCKTSGYTFTRYTMHWVKKRPGQGLEWIGYINPSRGYTNYNQKFCDKATLTTDKSSSTAYMQLSSLTSEDSAVYYCARYYDDHYSLDYWGQGTTLTVSS

SEQ ID NO: 16 - protein, 103 aa, Artificial Sequence
  bsAb HC_K64C, anti-CD3 CH1
ASTKGPSVFPLAPSSKSTSGGTAALGCLVKDYFPEPVTVSWNSGALTSGVHTFPAVLQSSGLYSLSSVVKVPSSSLGTQTYICNVNHKPSNTKVDKKVEPKSC

SEQ ID NO: 17 - protein, 106 aa, Artificial Sequence
  bsAb HC_K64C, anti-CD3 VL
DIQLTQSPAIMSASPGEKVTMTCRASSSVSYMNWYQEKSGTSPKRWIYDTSKVASGVPYRFSGSGSGTSYSLTISSMEAEDAATYYCQQWSSNPLTFGAGTKLELK

SEQ ID NO: 18 - protein, 107 aa, Artificial Sequence
  bsAb HC_K64C, anti-CD3 CL
RTVAAPSVFIFPPSDEQLKSGTASVVCLLNNFYPREAKVQWKVDNALQSGNSQESVTEQDSKESTYSLSSTLTLSKADYEKHKVYACEVTHQGLSSPVTKSFNRGEC

SEQ ID NO: 19 - protein, 119 aa, Artificial Sequence
  bsAb HC_T120C, anti-CD3 VH
DIKLQQSGAELARPGASVKMSCKTSGYTFTRYTMHWVKKRPGQGLEWIGYINPSRGYTNYNQKFKDKATLTTDKSSSTAYMQLSSLTSEDSAVYYCARYYDDHYSLDYWGQGTTLTVSS

SEQ ID NO: 20 - protein, 103 aa, Artificial Sequence
  bsAb HC_T120C, anti-CD3 CH1
ASCKGPSVFPLAPSSKSTSGGTAALGCLVKDYFPEPVTVSWNSGALTSGVHTFPAVLQSSGLYSLSSVVKVPSSSLGTQTYICNVNHKPSNTKVDKKVEPKSC

SEQ ID NO: 21 - protein, 106 aa, Artificial Sequence; identical to SEQ ID NO: 17
  bsAb HC_T120C, anti-CD3 VL

SEQ ID NO: 22 - protein, 107 aa, Artificial Sequence; identical to SEQ ID NO: 18
  bsAb HC_T120C, anti-CD3 CL

SEQ ID NO: 23 - protein, 118 aa, Artificial Sequence
  Anti-DLL3 VH
EVRLSQSGGQMKKPGESMRLSCRASGYTFTSYVMHWVREAPGRRPEWIGYINPYNDATKYARKFQGRATLTSDKYSDTAFLELRSLTSDDTAVYYCARGGYDYDGDYWGRGAPVTVSS

SEQ ID NO: 24 - protein, 103 aa, Artificial Sequence
  Anti-DLL3 CH1
ASTKGPSVFPLAPSSCSTSGGTAALGCLVKDYFPEPVTVSWNSGALTSGVHTFPAVLQSSGLYSLSSVVEVPSSSLGTQTYICNVNHKPSNTKVDKKVEPKSS

SEQ ID NO: 25 - protein, 107 aa, Artificial Sequence
  Anti-DLL3 VL
EIVLTQSPGTLSLSPGERATLSCHASQNINVWLSWYQKKPGQAPRLLIYKASNLHTGIPDRFSGSGSGTDFTLTISRLEPEDFAVYYCQQGQSYPFTFGQGTKVEIK

SEQ ID NO: 26 - protein, 107 aa, Artificial Sequence
  Anti-DLL3 CL
RTVAAPSVFIFPPSDEQLKSGTASVVCLLNNFYPREAKVQWKVDNALQSGNSQESVTEQDSKKSTYSLSSTLTLSKADYEKHKVYACEVTHQGLSSPVTKSCNRGES

SEQ ID NO: 27 - protein, 119 aa, Artificial Sequence
  #2 Anti-CD3 VH
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 28 - protein, 106 aa, Artificial Sequence [sequence omitted]

SEQ ID NO: 29 - protein, 119 aa, Artificial Sequence
  bsAb HC_K64C, #2 anti-CD3 VH
QVQLQQSGAELARPGASVKMSCKASGYTFTRYTMHWVKKRPGQGLEWIGYINPSRGYTNYNQKFCDKATLTTDKSSSTAYMQLSSLTSEDSAVYYCARYYDDHYSLDYWGQGTTLTVSS

SEQ ID NO: 30 - protein, 106 aa, Artificial Sequence
  bsAb HC_K64C, #2 anti-CD3 VL
QIVLTQSPAIMSASPGEKVTMTCSASSSVSYMNWYQEKSGTSPKRWIYDTSKLASGVPAHFRGSGSGTSYSLTISGMEAEDAATYYCQQWSSNPFTFGSGTKLEIN

SEQ ID NO: 31 - protein, 119 aa, Artificial Sequence
  bsAb HC_T120C, #2 anti-CD3 VH
QVQLQQSGAELARPGASVKMSCKASGYTFTRYTMHWVKKRPGQGLEWIGYINPSRGYTNYNQKFKDKATLTTDKSSSTAYMQLSSLTSEDSAVYYCARYYDDHYSLDYWGQGTTLTVSS

SEQ ID NO: 32 - protein, 106 aa, Artificial Sequence; identical to SEQ ID NO: 30
  bsAb HC_T120C, #2 anti-CD3 VL

SEQ ID NO: 33 - protein, 10 aa, Artificial Sequence
  #2 Anti-CD3 HCDR1
GYTFTRYTMH

SEQ ID NO: 34 - protein, 17 aa, Artificial Sequence
  #2 Anti-CD3 HCDR2
YINPSRGYTNYNQKFKD

SEQ ID NO: 35 - protein, 12 aa, Artificial Sequence
  #2 Anti-CD3 HCDR3
ARYYDDHYSLDY

SEQ ID NO: 36 - protein, 10 aa, Artificial Sequence
  #2 Anti-CD3 LCDR1
SASSSVSYMN

SEQ ID NO: 37 - protein, 7 aa, Artificial Sequence
  #2 Anti-CD3 LCDR2
DTSKLAS

SEQ ID NO: 38 - protein, 9 aa, Artificial Sequence
  #2 Anti-CD3 LCDR3
QQWSSNPFT